(19) United States
(12) Patent Application Publication
(10) Pub. No.: US 2010/0160351 A1
(43) Pub. Date: Jun. 24, 2010

(54) PHARMACEUTICAL COMPOSITIONS AND METHODS FOR TREATING HYPERURICEMIA AND RELATED DISORDERS

(75) Inventors: Helen JENKINS, Foster City, CA (US); Michael KITT, Mountain View, CA (US); Rodney PEARLMAN, El Granada, CA (US); Tito SERAFINI, Belmont, CA (US); Eugene THORSETT, Half Moon Bay, CA (US)

Correspondence Address: COOLEY GODWARD KRONISH LLP;ATTN: Patent Group, Suite 1100, 777 - 6th Street, NW, WASHINGTON, DC, 20001, US

(73) Assignee: NUON THERAPEUTICS, INC., San Diego, CA (US)

(21) Appl. No.: 12/642,802

(22) Filed: Dec. 19, 2009

Related U.S. Application Data
(60) Provisional application No. 61/140,802, filed on Dec. 24, 2008.
(60) Provisional application No. 61/161,754, filed on Mar. 19, 2009.
(60) Provisional application No. 61/165,114, filed on Mar. 31, 2009.
(60) Provisional application No. 61/242,354, filed on Sep. 14, 2009.
(60) Provisional application No. 61/232,344, filed on Aug. 7, 2009.
(60) Provisional application No. 61/139,415, filed on Dec. 19, 2008.

(51) Int. Cl. A61K 31/519; A61K 31/426; A61K 31/196; A61P 19/06

(52) U.S. Cl. 514/262.1; 514/365; 514/563

(57) ABSTRACT

Disclosed is a pharmaceutical composition comprising (a) a first therapeutic agent, wherein the first therapeutic agent is a compound of formula II:

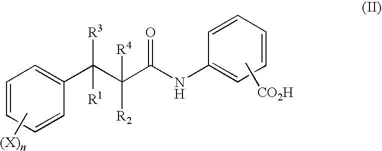

or a pharmaceutically acceptable salt thereof, wherein R¹, R², R³, R⁴, X and n are as defined herein; (b) a second therapeutic agent, wherein the second therapeutic agent is a uric acid synthesis inhibitor or a uricosuric agent; and (c) a pharmaceutically acceptable diluent or carrier.

### RELATED APPLICATIONS

This application claims the benefit of U.S. Provisional Application No. 61/139,415, filed Dec. 19, 2008, U.S. Provisional Application No. 61/140,802, filed Dec. 24, 2008, U.S. Provisional Application No. 61/161,754, filed Mar. 19, 2009, U.S. Provisional Application No. 61/165,114, filed Mar. 31, 2009, U.S. Provisional Application No. 61/242,354, filed Sep. 14, 2009, and U.S. Provisional Application No. 61/242,344, filed Sep. 14, 2009, each of which is hereby incorporated be reference in its entirety.

### BACKGROUND OF THE INVENTION

Gout, which is sometimes called podagre, affects 3 to 5 million individuals in the United States and continues to increase in incidence. Gout includes a group of disorders including painful attacks of acute, monarticular, inflammatory arthritis due to uric acid crystals, deposition of urate crystals in joints, deposition of urate crystals in renal parenchyma, urolithiasis (formation of calculus in the urinary tract), and nephrolithiasis (formation of kidney stones). Gouty arthritis is usually an extremely painful attack of gout with a rapid onset of joint inflammation. The joint inflammation is precipitated by deposits of uric-acid crystals in the joint fluid (synovial fluid) and joint lining (synovial lining). Intense joint inflammation occurs as white blood cells engulf the uric-acid crystals and release chemicals of inflammation, causing pain, heat, and redness of the joint tissues. Chronic gout can lead to deposits of hard lumps of uric acid in and around the joints, kidney stones, and blockage of the kidney-filtering tubules with uric-acid crystals, leading to kidney failure.

The underlying metabolic aberration in gout is hyperuricemia. Hyperuricemia has been associated with a serum uric acid (sUA) level of 6.8 mg/dL or greater, which is the upper limit of solubility of uric acid (also called urate) in extracellular fluids. However, hyperuricemia also has been associated with other levels of serum uric acid depending on factors such as gender and age, for example. Hyperuricemia leads to gout when urate crystals are formed from supersaturated body fluids and deposited in joints, tophi, and parenchymal organs.

In addition to gout, other disorders related to elevated serum uric acid levels include gout-associated inflammation, renal disorders, cardiovascular disease, aberrant metabolic conditions, fatty liver disease, kidney stones, cognitive impairment and dementia. Elevated serum uric acid levels have been identified as an independent risk factor for chronic kidney disease, cardiovascular disease and hypertension. Edwards, N. L., Clev. Clin. J. Med., Vol. 75, Suppl. 5, July 2008, S13-16. With respect to cardiovascular disease, hyperuricemia has been identified as an independent risk factor for athersclerotic disease in general and for coronary artery disease in particular. Edwards, N. L., Curr. Opin. Rheum., 2009, 21:132-137. It also has been identified as an independent risk factor for heart failure. Krishnan, E., Circ. Heart Fail., 2009, 2:556-562. Hyperuricemia independent of crystal deposition also may play a pathogenetic role in aberrant metabolic states, such as hypertriglyceridemia, obesity, insulin resistance, diabetes and metabolic syndrome. Beck, M. A., et. al, Rheum Dis. Clin. Am., 32 (2006), 275-293. In addition, hyperuricemia has been linked to cerebral vascular disease. Edwards, N. L., Curr. Opin. Rheum., 2009, 21:132-137. Elevated serum uric acid also has been independently associated with non-alcoholic fatty liver disease (NAFLD). Lee, Y-J., et. al, Clin. Chem. Lab. Med., 2010, 48(2). Elevated serum uric acid also has been identified as a strong risk factor for myocardial infarction and stroke. Bos, M. J., et al., Stroke, 2006, 37, 1503-1507. In addition, normal but mildly elevated serum uric acid levels have been linked to cognitive impairment and dementia. Schretlen, D. J., et al., Neuropsychology, 2007, Vol. 21, No. 1, 1.36-140.

The biosynthesis pathway for uric acid is represent in the following Scheme I:

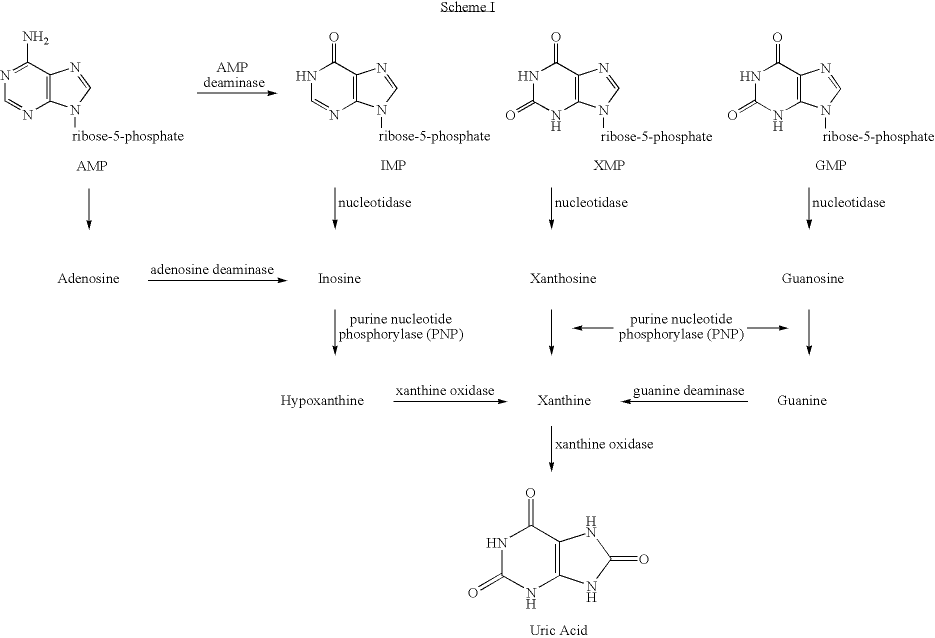

Scheme I. Purines are converted to hypoxanthine, then xanthine and finally urate via sequential oxidation by the enzyme xanthine oxidase.

Hyperuricemia can result from increased production or decreased excretion of uric acid, or from a combination of the two processes. Urate is the end product of purine metabolism in humans, shown above in Scheme I.

Known methods for treating gout include the use of uric acid synthesis inhibitors to inhibit the accumulation of uric acid in the body. These compounds function by inhibiting an enzyme involved in uric acid synthesis. In fact, it may be possible to inhibit uric acid synthesis by inhibiting any one of several enzymes shown above to be involved in uric acid synthesis. For example, xanthine oxidase inhibitors, such as febuxostat and allopurinol, reduce serum uric acid levels by inhibiting the enzyme xanthine oxidase. Known methods also include introduction of a recombinant, non-human uricase enzyme into the body, such as rasburicase or pegloticase.

Another known method for treating gout involves the use of uric acid excretion promoters These compounds accelerate the rapid excretion of uric acid accumulated in the body. Probenecid, sulfinpyrazone and benzbromarone are examples of uric acid excretion promoters. These compounds prevent the reuptake of urate back into the bloodstream in the kidney, leading to a net increase in excretion. Interleukin-6 (IL-6) has been proposed for use in the treatment of gout as a serum uric acid decreasing agent (see U.S. Pat. No. 6,007,804). In addition, non-steroidal anti-inflammatory drugs (NSAIDs), corticosteroids and colchicine have been used to treat some of the painful symptoms associated with gout.

However, the previously used anti-hyperuricemia agents all have different side effects or toxicity, such as the deposition of urate crystal in the urethra, leading to renal dysfunction and renal colic.

Therefore, it is necessary to find new pharmaceutical compositions and methods for treating gout, hyperuricemia and related disorders, and for lowering serum uric acid levels.

### SUMMARY OF THE INVENTION

In one aspect the present invention is directed to a pharmaceutical composition comprising:

(a) a first therapeutic agent, wherein said first therapeutic agent is a compound of formula II:

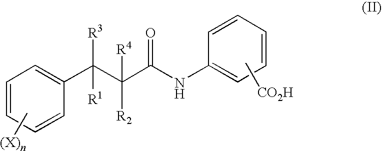

or a pharmaceutically acceptable salt thereof, wherein each of R¹ and R² is independently selected from a hydrogen atom or a C₁-C₄ alkyl group, R³ and R⁴ are each hydrogen atoms or together form another chemical bond, each X is independently selected from a hydroxyl group, a halogen atom, a C₁-C₄ alkyl group or a C₁-C₄ alkoxy group, or when two X groups are alkyl or alkoxy groups, they may be connected together to form a ring, and n is an integer from 1 to 3; (b) a second therapeutic agent, wherein said second therapeutic agent is a uric acid synthesis inhibitor or a uricosuric agent; and (c) a pharmaceutically acceptable diluent or carrier.

In another aspect the present invention provides a method of treating a condition associated with an elevated serum uric acid level comprising administering to a subject in need thereof a pharmaceutical composition comprising (a) a first therapeutic agent, wherein said first therapeutic agent is a compound of formula II, or a pharmaceutically acceptable salt thereof; (b) a second therapeutic agent, wherein said second therapeutic agent is a uric acid synthesis inhibitor or a uricosuric agent; and (c) a pharmaceutically acceptable diluent or carrier.

In another aspect the present invention provides a method of treating a condition associated with an elevated serum uric acid level comprising administering to a subject in need thereof of a pharmaceutically effective amount of compound of formula II, or a pharmaceutically acceptable salt thereof.

Another aspect of the present invention provides a method of decreasing serum uric acid level in a subject having a condition associated with an elevated serum uric acid level comprising administering to a subject in need thereof a pharmaceutical composition comprising (a) a first therapeutic agent, wherein said first therapeutic agent is a compound of formula II, or a pharmaceutically acceptable salt thereof; (b) a second therapeutic agent, wherein said second therapeutic agent is a uric acid synthesis inhibitor or a uricosuric agent; and (c) a pharmaceutically acceptable diluent or carrier.

Another aspect of the present invention provides a method of decreasing serum uric acid level in a subject having a condition associated with an elevated serum uric acid level comprising administering to a subject in need thereof a pharmaceutically effective amount of a compound of formula II, or a pharmaceutically acceptable salt thereof.

In another aspect the present invention provides a method of decreasing serum uric acid level in a subject comprising administering to a subject in need thereof a pharmaceutical composition comprising (a) a first therapeutic agent, wherein said first therapeutic agent is a compound of formula II, or a pharmaceutically acceptable salt thereof; (b) a second therapeutic agent, wherein said second therapeutic agent is a uric acid synthesis inhibitor or a uricosuric agent; and (c) a pharmaceutically acceptable diluent or carrier.

In another aspect the present invention provides a method of decreasing serum uric acid level in a subject comprising administering to a subject in need thereof a pharmaceutically effective amount of a compound of formula II, or a pharmaceutically acceptable salt thereof.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

A better understanding of the features and advantages of the present invention will be obtained by reference to the following detailed description that sets forth illustrative embodiments, in which the principles of the invention are utilized, and the accompanying drawing of which:

FIG. 1 shows the effects of Tranilast on serum uric acid levels in hyperuricemic patients. All patients had uric acid baseline levels equal to or above 8 mg/dL. Tranilast was administered twice daily for one or three months at the indicated dosages.

### DETAILED DESCRIPTION OF THE INVENTION

In one embodiment the present invention provides a pharmaceutical composition comprising:

(a) a first therapeutic agent, wherein said first therapeutic agent is a compound of formula II:

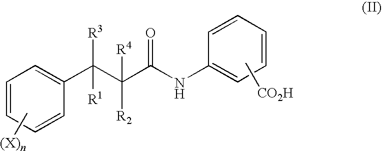

or a pharmaceutically acceptable salt thereof, wherein each of R¹ and R² is independently selected from a hydrogen atom or a C₁-C₄ alkyl group, R³ and R⁴ are each hydrogen atoms or together form another chemical bond, each X is independently selected from a hydroxyl group, a halogen atom, a C₁-C₄ alkyl group or a C₁-C₄ alkoxy group, or when two X groups are alkyl or alkoxy groups, they may be connected together to form a ring, and n is an integer from 1 to 3; (b) a second therapeutic agent, wherein said second therapeutic agent is a uric acid synthesis inhibitor or a uricosuric agent; and (c) a pharmaceutically acceptable diluent or carrier.

In another embodiment the present invention provides a method of treating a condition associated with an elevated serum uric acid level comprising administering to a subject in need thereof a pharmaceutical composition comprising (a) a first therapeutic agent, wherein said first therapeutic agent is a compound of formula II, or a pharmaceutically acceptable salt thereof; (b) a second therapeutic agent, wherein said second therapeutic agent is a uric acid synthesis inhibitor or a uricosuric agent; and (c) a pharmaceutically acceptable diluent or carrier.

In another embodiment the present invention provides a method of treating a condition associated with an elevated serum uric acid level comprising administering to a subject in need thereof a pharmaceutically effective amount of compound of formula II, or a pharmaceutically acceptable salt thereof.

In another embodiment the present invention provides a method of decreasing serum uric acid level in a subject having a condition associated with an elevated serum uric acid level comprising administering to a subject in need thereof a pharmaceutical composition comprising (a) a first therapeutic agent, wherein said first therapeutic agent is a compound of formula II, or a pharmaceutically acceptable salt thereof; (b) a second therapeutic agent, wherein said second therapeutic agent is a uric acid synthesis inhibitor or a uricosuric agent; and (c) a pharmaceutically acceptable diluent or carrier.

In another embodiment the present invention provides a method of decreasing serum uric acid level in a subject having a condition associated with an elevated serum uric acid level comprising administering to a subject in need thereof a pharmaceutically effective amount of a compound of formula II, or a pharmaceutically acceptable salt thereof.

In still another embodiment the present invention provides a method of decreasing serum uric acid level in a subject comprising administering to a subject in need thereof a pharmaceutical composition comprising (a) a first therapeutic agent, wherein said first therapeutic agent is a compound of formula II, or a pharmaceutically acceptable salt thereof; (b) a second therapeutic agent, wherein said second therapeutic agent is a uric acid synthesis inhibitor or a uricosuric agent; and (c) a pharmaceutically acceptable diluent or carrier.

In still another embodiment the present invention provides a method of decreasing serum uric acid level in a subject comprising administering to a subject in need thereof a pharmaceutically effective amount of a compound of formula II, or a pharmaceutically acceptable salt thereof.

In still another embodiment the present invention provides a method of decreasing serum uric acid level in a subject that has experienced insufficient lowering of serum uric acid following treatment with a uric acid synthesis inhibitor, a uricosuric agent and/or a recombinant uricase enzyme.

In one embodiment the compound of formula II is a compound selected from the group consisting of: 2-[[3-(2-methylphenyl)-1-oxo-2-propenyl]amino]benzoic acid; 2-[[3-(3-methylphenyl)-1-oxo-2-propenyl]amino]benzoic acid; 2-[[3-(4-methylphenyl)-1-oxo-2-propenyl]amino]benzoic acid; 2-[[3-(2-ethylphenyl)-1-oxo-2-propenyl]amino]benzoic acid; 2-[[3-(3-ethylphenyl)-1-oxo-2-propenyl]amino]benzoic acid; 2-[[3-(4-ethylphenyl)-1-oxo-2-propenyl]amino]benzoic acid; 2-[[3-(2-propylphenyl)-1-oxo-2-propenyl]amino]benzoic acid; 2-[[3-(3-propylphenyl)-1-oxo-2-propenyl]amino]benzoic acid; 2-[[3-(4-propylphenyl)-1-oxo-2-propenyl]amino]benzoic acid; 2-[[3-(2-hydroxyphenyl)-1-oxo-2-propenyl]amino]benzoic acid; 2-[[3-(3-hydroxyphenyl)-1-oxo-2-propenyl]amino]benzoic acid; 2-[[3-(4-hydroxyphenyl)-1-oxo-2-propenyl]amino]benzoic acid; 2-[[3-(2-chlorophenyl)-1-oxo-2-propenyl]amino]benzoic acid; 2-[[3-(3-chlorophenyl)-1-oxo-2-propenyl]amino]benzoic acid; 2-[[3-(4-chlorophenyl)-1-oxo-2-propenyl]amino]benzoic acid; 2-[[3-(2-fluorophenyl)-1-oxo-2-propenyl]amino]benzoic acid; 2-[[3-(3-fluorophenyl)-1-oxo-2-propenyl]amino]benzoic acid; 2-[[3-(4-fluorophenyl)-1-oxo-2-propenyl]amino]benzoic acid; 2-[[3-(2-bromophenyl)-1-oxo-2-propenyl]amino]benzoic acid; 2-[[3-(3-bromophenyl)-1-oxo-2-propenyl]amino]benzoic acid; 2-[[3-(4-bromophenyl)-1-oxo-2-propenyl]amino]benzoic acid; 2-[[3-(2,3-dimethoxyphenyl)-1-oxo-2-propenyl]amino]benzoic acid; 2-[[3-(3,4-dimethoxyphenyl)-1-oxo-2-propenyl]amino]benzoic acid; 2-[[3-(2,4-dimethoxyphenyl)-1-oxo-2-propenyl]amino]benzoic acid; 2-[[3-(2,3-dimethylphenyl)-1-oxo-2-propenyl]amino]benzoic acid; 2-[[3-(3,4-dimethylphenyl)-1-oxo-2-propenyl]amino]benzoic acid; 2-[[3-(2,4-dimethylphenyl)-1-oxo-2-propenyl]amino]benzoic acid; 2-[[3-(2,3-diethoxyphenyl)-1-oxo-2-propenyl]amino]benzoic acid; 2-[[3-(3,4-diethoxyphenyl)-1-oxo-2-propenyl]amino]benzoic acid; 2-[[3-(2,4-diethoxyphenyl)-1-oxo-2-propenyl]amino]benzoic acid; 2-[[3-(2,3-dipropoxyphenyl)-1-oxo-2-propenyl]amino]benzoic acid; 2-[[3-(3,4-dipropoxyphenyl)-1-oxo-2-propenyl]amino]benzoic acid; 2-[[3-(2,4-dipropoxyphenyl)-1-oxo-2-propenyl]amino]benzoic acid; 2-[[3-(2,3-diethylphenyl)-1-oxo-2-propenyl]amino]benzoic acid; 2-[[3-(3,4-diethylphenyl)-1-oxo-2-propenyl]amino]benzoic acid; 2-[[3-(2,4-diethylphenyl)-1-oxo-2-propenyl]amino]benzoic acid; 2-[[3-(2,3-dipropylphenyl)-1-oxo-2-propenyl]amino]benzoic acid; 2-[[3-(3,4-dipropylphenyl)-1-oxo-2-propenyl]amino]benzoic acid; 2-[[3-(2,4-dipropylphenyl)-1-oxo-2-propenyl]amino]benzoic acid; 2-[[3-(2-methoxy-3-methylphenyl)-1-oxo-2-propenyl]amino]benzoic acid, 2-[[3-(3-methoxy-4-methylphenyl)-1-oxo-2-propenyl]amino]benzoic acid; 2-[[3-(2-methoxy-3-methylphenyl)-1-oxo-2-propenyl]amino]benzoic acid; 2-[[3-(2-methoxy-4-methylphenyl)-1-oxo-2-propenyl]amino]benzoic acid; 2-[[3-(2-methoxy-3-chlorophenyl)-1-oxo-2-propenyl]amino]benzoic acid; 2-[[3-(3-methoxy-4-chlorophenyl)-1-oxo-2-propenyl]amino]benzoic acid; 2-[[3-(2-methoxy-3-chlorophenyl)-1-oxo-2-propenyl]amino]benzoic acid; 2-[[3-(2-methoxy-4-chlorophenyl)-1-oxo-2-propenyl]amino]benzoic acid; 2-[[3-(2-methoxy-3-hydroxyphenyl)-1-oxo-2-propenyl]amino]benzoic acid; 2-[[3-(3-methoxy-4-hydroxyphenyl)-1-oxo-2-propenyl]amino]benzoic acid; 2-[[3-(2-methoxy-3-hydroxyphenyl)-1-oxo-2-propenyl]amino]benzoic acid; 2-[[3-(2-methoxy-4-hydroxyphenyl)-1-oxo-2-propenyl]amino]benzoic acid; 2-[[3-(3,4-trimethylenephenyl)-1-oxo-2-propenyl]amino]benzoic acid; 2-[[3-(2,3-trimethylenephenyl)-1-oxo-2-propenyl]amino]benzoic acid; 2-[[3-(3,4-methylenedioxyphenyl)-1-oxo-2-propenyl]amino]benzoic acid; and 2-[[3-(3,4-ethylenedioxyphenyl)-1-oxo-2-propenyl]amino]benzoic acid.

In one embodiment the compound of formula II is 2-[[3-(3,4-dimethoxyphenyl)-1-oxo-2-propenyl]amino]benzoic acid (tranilast).

In one embodiment the second therapeutic agent is a uric acid synthesis inhibitor. In one such embodiment the uric acid synthesis inhibitor is a xanthine oxidase inhibitor. In one such embodiment the xanthine oxidase inhibitor is allopurinol, febuxostat, oxypurinol, tisopurine or an inositol. In one such embodiment the xanthine oxidase inhibitor is allopurinol or febuxostat. In one such embodiment the xanthine oxidase inhibitor is allopurinol. In another such embodiment the xanthine oxidase inhibitor is febuxostat.

In one embodiment, the pharmaceutical composition comprises from about 100 mg to about 300 mg of tranilast and from about 100 mg to about 300 mg of allopurinol. In one such embodiment, the pharmaceutical composition comprises about 100 mg, about 150 mg, about 200 mg, about 250 mg or about 300 mg of tranilast and about 100 mg, about 150 mg, about 200 mg, about 250 mg or about 300 mg of allopurinol. In one such embodiment the pharmaceutical composition comprises about 300 mg of tranilast and about 300 mg of allopurinol.

In another embodiment, the second therapeutic agent is a uricosuric agent. In one such embodiment the uricosuric agent is probenecid, benzbromarone, sulfinpyrazone, guaifenesin, losartan, atorvastatin, amlodipine, adrenocorticotropic hormone or fenofibrate. In one such embodiment, the uricosuric agent is probenecid.

In one embodiment, the condition associated with an elevated serum uric acid level is hyperuricemia, gout, a renal disorder, cardiovascular disease, an aberrant metabolic condition, cognitive impairment, a fatty liver disease or kidney stones.

In other embodiments, where the present invention provides methods for decreasing serum uric acid level in a subject, the subject has hyperuricemia, gout, gout-associated inflammation, a renal disorder, cardiovascular disease, an aberrant metabolic condition, cognitive impairment, fatty liver disease or kidney stones. In one such embodiment, the subject has cognitive impairment. Cognitive impairment may be associated with cerebral vascular conditions, Alzheimer's disease, Parkinson's disease or aging. Schretlen, D. J., et al., Neuropsychology, 2007, Vol. 21, No. 1, 136-140.

In one aspect the condition associated with an elevated serum uric acid level is hyperuricemia. In one such aspect the method comprises reducing inflammation associated with hyperuricemia.

In one aspect the present invention provides methods for treating hyperuricemia in a subject with a condition selected from the group of gout, a renal disorder, cardiovascular disease, an aberrant metabolic condition, cognitive impairment, a fatty liver disease and kidney stones. In one such embodiment, the subject has gout.

In another aspect the condition associated with an elevated serum uric acid level is gout. In one such aspect the method comprises treating gouty symptoms. In another such aspect the method comprises treating gouty attacks. In another such aspect the method comprises reducing the incidence and/or severity of gouty flares. In another such aspect the method comprises preventing, reducing or reversing uric acid crystal formation. In another such embodiment the method comprises reducing uric acid burden. In another such aspect the method comprises reducing the size and/or number of tophi. The size and/or number of tophi may be assessed by, for example, use of CT scans.

In yet another aspect of the present invention the condition associated with an elevated serum uric acid level is a renal disorder. In one such aspect the renal disorder is chronic kidney disease.

In yet another aspect of the present invention the condition associated with an elevated serum uric acid level is kidney stones.

In yet another aspect of the present invention the condition associated with an elevated serum uric acid level is cardiovascular disease. In one such aspect the cardiovascular disease is coronary artery disease, stroke, peripheral artery disease, congestive heart failure or hypertension. In one such aspect the cardiovascular disease is coronary heart disease. In another such aspect the cardiovascular disease is stroke. In yet another such aspect the cardiovascular disease is peripheral artery disease. In yet another such aspect the cardiovascular disease is congestive heart failure. In yet another such aspect the cardiovascular disease is hypertension.

In yet another aspect of the present invention the condition associated with an elevated serum uric acid level is an aberrant metabolic condition. In one such aspect the aberrant metabolic condition is metabolic syndrome, obesity, hyperlipidemia, insulin resistance or diabetes. In one such aspect the aberrant metabolic condition is metabolic syndrome. In another such aspect the aberrant metabolic condition is obesity. In yet another such aspect the aberrant metabolic condition is hyperlipidemia. In yet another such aspect the aberrant metabolic condition is insulin resistance. In yet another such aspect the aberrant metabolic condition is diabetes.

In yet another aspect of the present invention the condition associated with an elevated serum uric acid level is cognitive impairment.

In yet another aspect of the present invention the condition associated with an elevated serum uric acid level is a fatty liver disease. In one such embodiment the fatty liver disease is non-alcoholic fatty liver disease (NAFLD). In another such embodiment, the non-alcoholic fatty liver disease is non-alcoholic steatohepatitis (NASH).

In certain embodiments, administration of a pharmaceutical composition of the present invention or administration of a compound of formula II or a pharmaceutically acceptable salt thereof to a subject in accordance with a method of the present invention decreases a serum uric acid level in the subject by at least about 5%, at least about 10%, at least about 20%, at least about 30%, at least about 40%, at least about 50%, at least about 60%, at least about 70%, at least about 80% or at least about 90%. In certain embodiments, the serum uric acid level in the subject is decreased by at least about 33%. In certain embodiments, the serum uric acid level in the subject is decreased by at least about 50%.

In certain embodiments, administration of a pharmaceutical composition of the present invention or administration of a compound of formula II or a pharmaceutically acceptable salt thereof to a subject in accordance with a method of the present invention decreases a serum uric acid level in the subject by from about 5% to about 90%, by from about 10% to about 50%, by from about 20% to about 40%, or by from about 25% to about 35%.

In further embodiments, the methods of the present invention comprise administering a pharmaceutical composition of the present invention or a compound of formula II to a subject whose serum uric acid level is at least about 4.0 mg/dL, at least about 4.5 mg/dL, at least about 5.0 mg/dL, at least about 5.5 mg/dL, at least about 6.0 mg/dL, at least about 6.5 mg/dL, at least about 6.8 mg/dL, at least about 7.0 mg/dL, at least about 7.5 mg/dL, at least about 8.0 mg/dL, at least about 8.5 mg/dL, at least about 9.0 mg/dL, at least about 9.5 mg/dL, at least about 10.0 mg/dL, at least about 10.5 mg/dL or at least about 11.0 mg/dL.

In further embodiments, the methods of the present invention decrease a serum uric acid level in the subject below about 7.0 mg/dL, 6.5 mg/dL, below about 6.0 mg/dL, below about 5.5 mg/dL, below about 5.0 mg/dL, below about 4.5 mg/dL, below about 4.0 mg/dL, below about 3.5 mg/dL, below about 3.0 mg/dL, below about 2.5 mg/dL, below about 2.0 mg/dL or below about 1.5 mg/dL. The appropriate serum uric acid level may vary depending on the subject, and may vary for a given subject over time, depending upon the subject's overall medical condition. Similarly, the appropriate serum uric acid level for one group of subjects sharing a common medical condition may be different from that which is appropriate for a different group of subjects sharing a different medical condition. Thus, it may be advisable to reduce the serum uric acid level of a given group of subjects to, for example, below about 5.0 mg/dL, and to reduce the serum uric acid level of a different group of subjects to, for example, below about 4.0 mg/dL. In certain embodiments, the methods of the present invention decrease a serum uric acid level in the subject by an amount sufficient to cause the disappearance of tophi over a timeframe of weeks or months.

In some embodiments, a serum uric acid level in the subject is decreased by between about 0.1 to about 10.0 mg/dL. In certain such embodiments a serum uric acid level in the subject is decreased by between about 0.5 to about 8.0 mg/dL, by between about 1.0 to about 6.0 mg/dL, or by between about 2.0 to about 5.0 mg/dL. In certain other embodiment the serum uric acid level in the subject is decreased by between about 1.0 to about 4.0 mg/dL, or by between about 1.0 to about 2.0 mg/dL. Again, the amount of decrease of serum uric acid level that is appropriate may vary depending on the subject, depending upon the subject's overall medical condition. Similarly, the amount of decrease of serum uric acid level that is appropriate for one group of subjects sharing a common medical condition may be different from that which is appropriate for a different group of subjects sharing a different medical condition.

In certain embodiments, the methods of the present invention comprise administering a pharmaceutical composition of the present invention or a compound of formula II to a subject whose serum uric acid level is within the normal range.

In further embodiments of the methods for treating a condition associated with an elevated serum uric acid level, the subject has gout. In some embodiments, the subject has severe gout. In some embodiments, the subject has chronic gout. In some embodiments, the subject has acute gout. In some embodiments, the subject has refractory gout. In some embodiments, the subject has had at least one gouty attack. In some embodiments, the subject has uric acid crystal formation determined by aspiration of tophi or by aspiration of synovial fluid of an inflamed joint.

In further embodiments of the methods for treating a condition associated with an elevated serum uric acid level, the subject has a known risk of gouty attack. In some embodiments, the risk of gouty attack is determined by a combination of hyperuricemia and one or more of a history of gouty attack, obesity, diabetes, chronic kidney failure, hypertension, use of diuretic drugs, high purine diet, high fructose diet, exposure to lead, high consumption of red meat and protein, and high alcohol intake.

In some embodiments of the methods for treating gouty symptoms, the gouty symptoms comprise one or more of pain, inflammation, swelling, muscle fatigue, stress feelings, kidney stones, tophi, podagra or myocardial infarction. In one such embodiment the gouty symptoms are tophi.

In some embodiments of the methods for treating uric acid crystal formation, the uric acid crystal formation is in one or more of the joints, under skin, and kidney. In some embodiments, the formations include tophaceous deposits.

In some embodiments of the methods for treating kidney stones, the kidney stones comprise one or more of uric acid, calcium oxalate and calcium phosphate. In some embodiments, the kidney stones are caused by increased uric acid levels and formation of uric acid crystals.

In some embodiments of the methods for treating a renal disorder, the renal disorder is urinary lithiasis, hyperuricemic nephropathy, acute uric acid nephropathy, microalbuminuria, renal dysfunction, impaired glomerular filtration rate, or nephrolithiasis. In some embodiments, the renal disorder is renal insufficiency or chronic kidney disease. In one such embodiment the renal disorder is renal insufficiency. In another such embodiment the renal disorder is chronic kidney disease.

In some embodiments of the methods for treating cardiovascular disease, the cardiovascular disease is hypertension, myocardial infarction, coronary artery disease, cerebrovascular disease, vascular dementia, preeclampsia, heart disease, congestive heart failure, stroke, atherogenesis, thrombogenesis, atherosclerosis, inflammatory disease or peripheral, carotid, or coronary vascular disease. In one such embodiment the cardiovascular disease is hypertension. In another such embodiment the cardiovascular disease is coronary artery disease. In yet another such embodiment the cardiovascular disease is congestive heart failure. In yet another such embodiment the cardiovascular disease is stroke. In yet another such embodiment the cardiovascular disease is atherosclerosis. In yet another such embodiment the cardiovascular disease is peripheral vascular disease.

In some embodiments of the methods for treating an aberrant metabolic condition the aberrant metabolic condition is metabolic syndrome, obesity, hyperlipidemia, insulin resistance or diabetes. In one such embodiment the aberrant metabolic condition is metabolic syndrome. In another such embodiment the aberrant metabolic condition is obesity. In yet another such embodiment the aberrant metabolic condition is hyperlipidemia. In yet another such embodiment the aberrant metabolic condition is insulin resistance. In yet another such embodiment the aberrant metabolic condition is diabetes.

In some embodiments of the methods for treating cognitive impairment, the cognitive impairment is dementia or Alzheimer's disease.

In some embodiments of the methods for treating a fatty liver disease the fatty liver disease is non-alcoholic fatty liver disease (NAFLD). In one such embodiment, the non-alcoholic fatty liver disease is non-alcoholic steatohepatitis (NASH).

In some embodiments, where the present invention provides methods of lowering serum uric acid in a subject that has experienced insufficient lowering of serum uric acid following treatment with a uric acid synthesis inhibitor, uricosuric agent and/or a recombinant uricase, the uric acid synthesis inhibitor is a xanthine oxidase inhibitor. In one such embodiment the xanthine oxidase inhibitor is allopurinol. In another such embodiment the xanthine oxidase inhibitor is febuxostat.

In other embodiments, where the present invention provides methods of lowering serum uric acid in a subject that has experienced insufficient lowering of serum uric acid following treatment with a uric acid synthesis inhibitor and/or uricosuric agent, the uricosuric agent is probenecid. In another such embodiment the uricosuric agent is a uricase.

In any of the methods described herein, the methods can further comprise measuring serum uric acid levels in the subject before and after administration of a compound of the invention, wherein a decrease in serum uric acid levels after the administration indicates an effective treatment. In any of the methods described herein, the compound of formula II or pharmaceutically acceptable salt thereof, or the second therapeutic agent, can be administered at between about 10 mg per day and about 2000 mg per day. In any of the methods described herein, t the compound of formula II or pharmaceutically acceptable salt thereof, or the second therapeutic agent, can be administered at between about 50 mg per day and about 600 mg per day. In some embodiments, the compound of formula II or pharmaceutically acceptable salt thereof, or the second therapeutic agent, is, administered at about 50 mg per day. In some embodiments, the compound of formula II or pharmaceutically acceptable salt thereof, or the second therapeutic agent is administered at about 100 mg per day. In some embodiments, the compound of formula II or pharmaceutically acceptable salt thereof, or the second therapeutic agent, is administered at about 150 mg per day. In some embodiments, the compound of formula II or pharmaceutically acceptable salt thereof, or the second therapeutic agent, is administered at about 200 mg per day. In some embodiments, the compound of formula II or pharmaceutically acceptable salt thereof, or the second therapeutic agent, is administered at about 250 mg per day. In some embodiments, the compound of formula II or pharmaceutically acceptable salt thereof, or the second therapeutic agent, is administered at about 300 mg per day. In some embodiments, the compound of formula II or pharmaceutically acceptable salt thereof, or the second therapeutic agent, is administered at about 350 mg per day. In some embodiments, the compound of formula II or pharmaceutically acceptable salt thereof, or the second therapeutic agent, is administered at about 400 mg per day. In some embodiments, the compound of formula II or pharmaceutically acceptable salt thereof, or the second therapeutic agent, is administered at about 450 mg per day. In some embodiments, t the compound of formula II or pharmaceutically acceptable salt thereof, or the second therapeutic agent, is administered at about 500 mg per day. In some embodiments, the compound of formula II or pharmaceutically acceptable salt thereof, or the second therapeutic agent, is administered at about 550 mg per day. In some embodiments, the compound of formula II or pharmaceutically acceptable salt thereof, or the second therapeutic agent, is administered at about 600 mg per day. In some embodiments, the compound of formula II or pharmaceutically acceptable salt thereof, or the second therapeutic agent, is administered at about 650 mg per day. In some embodiments, the compound of formula II or pharmaceutically acceptable salt thereof, or the second therapeutic agent, is administered at about 700 mg per day. In some embodiments, the compound of formula II or pharmaceutically acceptable salt thereof, or the second therapeutic agent, is administered at about 750 mg per day. In some embodiments, the compound of formula II or pharmaceutically acceptable salt thereof, or the second therapeutic agent, is administered at about 800 mg per day. In some embodiments, the compound of formula II or pharmaceutically acceptable salt thereof, or the second therapeutic agent, is administered at about 850 mg per day. In some embodiments, the compound of formula II or pharmaceutically acceptable salt thereof, or the second therapeutic agent, is administered at about 900 mg per day. In some embodiments, the compound of formula II or pharmaceutically acceptable salt thereof, or the second therapeutic agent, is administered at about 950 mg per day. In some embodiments, the compound of formula II or pharmaceutically acceptable salt thereof, or the second therapeutic agent, is administered at about 1000 mg per day. In some embodiments, the compound of formula II or pharmaceutically acceptable salt thereof, or the second therapeutic agent, is administered at about 2000 mg per day.

In some aspects, the present invention provides a method of treating hyperuricemia in a subject with gout comprising administering Tranilast to the subject. In some embodiments, the Tranilast is administered at between about 50 mg and about 900 mg per day. In some such embodiments, Tranilast is administered at between about 100 mg and about 300 mg per day.

In certain embodiments of the methods for treating a condition associated with an elevated serum uric acid level, the subject is administered, in addition to a compound of formula II, or a pharmaceutically acceptable salt thereof (e.g., Tranilast), a second therapeutic agent sequentially or simultaneously. In the case of simultaneous administration, the compound of formula II, or a pharmaceutically acceptable salt thereof and second therapeutic agent may be administered in a single pharmaceutical composition, as described above.

In some embodiments, the second agent is a xanthine oxidase inhibitor. In some embodiments, the xanthine oxidase inhibitor is allopurinol, febuxostat, oxypurinol, tisopurine, or an inositol. In some embodiments, the second agent is a uricosuric agent. In some embodiments, the uricosuric agent is probenecid, benzbromarone, sulfinpyrazone, guaifenesin, losartan, atorvastatin, amlodipine, adrenocorticotropic hormone (ACTH or corticotropin), or fenofibrate. In some embodiments, the second agent is a uricase enzyme, or a fragment or pegylated derivative thereof. In some embodiments, the uricase enzyme is rasburicase or pegloticase. In some embodiments, the second agent is cortisone. In some embodiments, the second agent is an anti-inflammatory agent. In some embodiments, the anti-inflammatory agent is a nonsteroidal anti-inflammatory drug (NSAID). In some embodiments, the NSAID is diclofenac, indomethacin, naproxen, sulindac, lumiracoxib or a Cox-2 selective inhibitor. In some embodiments, the Cox-2 selective inhibitor is etoricoxib, celecoxib (SC-58635), 5-bromo-2-(4-fluorophenyl)-3-(4-(methylsulfonyl)phenyl)-thiophene (DUP-697), flosulide (CGP-28238), meloxicam, 6-methoxy-2 naphthylacetic acid (6-MNA), MK-966 (Vioxx), nabumetone (6-MNA prodrug), nimesulide, N-[2-(cyclohexyloxy)-4-nitrophenyl]-methanesulfonamide (NS-398), SC-5766, SC-58215, or 3-Formylamino-7-methylsulfonylamino-6-phenoxy-4H-1-benzopyran-1-one (T-614). In some embodiments, the anti-inflammatory agent is a corticosteroid. In some embodiments, the corticosteroid is methyl prednisolone, prednisolone, dexamethasone, fluticasone propionate, 6α,9α-difluoro-17-[(2-furanylcarbonyl)oxy]-11β-hydroxy-16α-methyl-3-oxo-androsta-1,4-diene-17β-carbothioic acid S-fluoromethyl ester, 6α,9α-difluoro-11β-hydroxy-16α-methyl-3-oxo-17.alpha.-propionyloxy-androsta-1,4-diene-17β-carbothioic acid S-(2-oxo-tetrahydro-furan-3S-yl)ester, beclomethasone esters, the 17-propionate ester or the 17,21-dipropionate ester, budesonide, flunisolide, mometasone esters, the furoate ester, triamcinolone acetonide, rofleponide, ciclesonide, butixocort propionate, RPR-106541, ST-126, fluticasone propionate, 6α,9α-difluoro-11β-hydroxy-16α-methyl-17α-[(-4-methyl-1,3-thiazole-5-carbonyl)oxy]-3-oxo-androsta-1,4-diene-17β-carbothioic acid S-fluoromethyl ester and 6α,9α-difluoro-17α-[(2-furanylcarbonyl)oxy]-11β-hydroxy-16α-methyl-3-oxo-androsta-1,4-diene-17β-carbothioic acid S-fluoromethyl ester, or 6α,9α-difluoro-17α-[(2-furanylcarbonyl)oxy]-11β-hydroxy-16α-methyl-3-oxo-androsta-1,4-diene-17β-carbothioic acid S-fluoromethyl ester. In some embodiments, the second agent is Colchicine or a prodrug thereof. In some embodiments, the second agent is an opioid agent. In some embodiments, the opioid agent is morphine, heroin, hydromorphone, oxymorphone, levorphanol, levallorphan, methadone, meperidine, fentanyl, cocaine, codeine, dihydrocodeine, oxycodone, hydrocodone, propoxyphene, nalmefene, nalorphine, naloxone, naltrexone, buprenorphine, butorphanol, nalbuphine or pentazocine. In some embodiments, the second agent is an IL-1 antagonist. In some embodiments, the IL-1 antagonist is Canakinumab (ACZ885) or Rilonacept (Arcalyst). In some embodiments, the second agent comprises IL-6 or a fragment thereof. In some embodiments, the second agent is insulin. In some embodiments, the second agent is selected from Table 2.

In some embodiments, the second therapeutic agent comprises a treatment for a cardiovascular disorder, diabetes, or obesity, or complications thereof. In certain such embodiments, the second therapeutic agent is glitazone, troglitazone, rosiglitazone (Avandia), pioglitazone, a sulphonylurea, gliquidone, tolbutamide, glimepride, chlorpropamide, glipizide, glyburide, acetohexamide, meglitinide, repaglinide, nateglinide, metformin, an endothelin receptor antagonist, bosentan, darusentan, enrasentan, tezosentan, atrasentan, ambrisentan sitaxsentan, a smooth muscle relaxant, a PDE5 inhibitor, minoxidil, an angiotensin converting enzyme (ACE) inhibitor, captopril, enalapril, lisinopril, fosinopril, perindopril, quinapril, trandolapril, benazepril, ramipril, a angiotensin II receptor blocker, irbesartan, losartan, valsartan, eprosartan, olmesartan, candesartan, telmisartan, a beta blocker, atenolol, metoprolol, nadolol, bisoprolol, pindolol, acebutolol, betaxolol, propranolol, a diuretic, thiazide, hydrochlorothiazide, furosemide, torsemide, metolazone, a calcium channel blocker, amlodipine, felodipine, nisoldipine, nifedipine, verapamil, diltiazem, a alpha receptor blocker, doxazosin, terazosin, alfuzosin, tamsulosin, a central alpha agonist, clonidine, a statin, atovastatin, fluvastatin, lovastatin, pravastatin, rosuvastatin calcium, simvastatin, nicotinic acid, a fibrate, gemfibrozil, fenofibrate, bezafibrate, ciprofibrate, a bile acid sequestrant, cholestyramine, colestipol, a cholesterol absorption inhibitor, a COX-1 inhibitor, aspirin, a NSAID, or a COX-2 inhibitor.

In some embodiments, the second therapeutic agent comprises a treatment for a renal disorder. In certain such embodiments, the second therapeutic agent is a NO donor, a calcium channel blocker, a cholinergic modulator, an alpha-adrenergic receptor antagonist, a beta-adrenergic receptor agonist, a phosphodiesterase inhibitor, a cAMP-dependent protein kinase activator, a cAMP mimetic, a superoxide scavenger, a potassium channel activator, an estrogen-like compound, a testosterone-like compound, a benzodiazepine, an adrenergic nerve inhibitor, an antidiarrheal agent, a HMG-CoA reductase inhibitor, a smooth muscle relaxant, a adenosine receptor modulator, an adenylyl cyclase activator, an endothelin receptor antagonist, a bisphosphonate, a cGMP-dependent protein kinase activator, a cGMP mimetic, an alpha adrenergic blocking agent, Flomax, Uroxatral, terazosin, doxazosin, a nonsteroidal anti-inflammatories, an opioid, codeine, hydrocodone, thiazide, potassium citrate, magnesium citrate, allopurinol, or calgranulin.

In further embodiments, the present invention provides a method of treating hyperuricemia in a subject with gout, cardiovascular disease, renal disease, a fatty liver disease, kidney stones or an aberrant metabolic condition comprising administering Tranilast to the subject in combination with a second therapeutic agent. In one such embodiment the method comprises treating hyperuricemia in a subject with gout. In another such embodiment the method comprises treating hyperuricemia in a subject with refractory gout. In another such embodiment the method comprises treating hyperuricemia in a subject with cardiovascular disease. In another such embodiment the method comprises treating hyperuricemia in a subject with renal disease. In another such embodiment the method comprises treating hyperuricemia in a subject with a fatty liver disease. In another such embodiment the method comprises treating hyperuricemia in a subject with kidney stones. In another such embodiment the method comprises treating hyperuricemia in a subject with an aberrant metabolic condition. In certain embodiments the second therapeutic agent is a xanthine oxidase inhibitor or a uricosuric agent. The effect of administering Tranilast to the subject in combination with one or more xanthine oxidase inhibitors or uricosuric agents may be additive or, in certain instances, more than additive. In some embodiments, the Tranilast is administered in combination with allopurinol. The daily dose of allopurinol may range from 50 mg to 900 mg, from 100 mg to 600 mg or from 100 mg to 300 mg. In some embodiments, the allopurinol is administered in 100 mg, 200 mg or 300 mg doses. In some embodiments, the Tranilast is administered in combination with febuxostat. The daily dose of febuxostat may range from 10 mg to 200 mg, from 20 mg to 120 mg or from 40 mg to 80 mg. In some embodiments, the febuxostat is administered in 40 mg or 80 mg doses. In certain embodiment, 300 mg of Tranilast is administered with 100 mg of allopurinol. In certain embodiments, 300 mg of Tranilast is administered with 200 mg of allopurinol. In certain embodiments. 300 mg of Tranilast is administered with 300 mg of allopurinol. In certain embodiment, 300 mg of Tranilast is administered with 20 mg of febuxostat. In certain embodiment, 300 mg of Tranilast is administered with 40 mg of febuxostat. In certain embodiment, 300 mg of Tranilast is administered with 80 mg of febuxostat.

In embodiments which provide a method for treating a condition associated with an elevated serum uric acid level in a subject by administering to the subject a first and a second therapeutic agent, either simultaneously or sequentially, the dose of the first and second therapeutic agents may be titrated. Thus, throughout the course of treatment, the amount of each therapeutic agent, as well as the weight ratio of the first therapeutic agent to second therapeutic agent, may vary as appropriate given the subject's condition and response to treatment.

The combination therapies disclosed herein can provide a beneficial therapeutic effect, particularly an additive or over-additive effect. In some embodiments the combination therapies disclosed herein can provide an overall reduction of side effects, e.g., adverse effects. In some embodiments the additive or over-additive beneficial therapeutic effect of the combination therapies disclosed herein provides for dose reduction and/or interval extension when compared to the isolated use of the individual therapeutic agents. Also, the effect of Tranilast on reducing the pain associated with inflammation may be of additional benefit during the arthritic flares associated with gout attacks, thus presenting a unique and differentiating therapy for the disease.

### I. Hyperuricemia

The present invention provides compositions and methods for treating hyperuricemia and related disorders. Hyperuricemia may be defined as a serum urate concentration greater than or equal to 6.8 mg/dL. At serum urate levels greater than or equal to 6.8 mg/dL, uric acid crystals can precipitate out of solution and deposit in joints and other body tissues where they can produce an inflammatory response, neutrophil recruitment, and the production of proinflammatory cytokines as well as other inflammatory mediators. In extremities where body temperatures may be lower than core body temperatures, uric acid crystals may precipitate at lower concentrations, such as 6.0 mg/dL or lower. Hyperuricemia may be due to overproduction of uric acid. For example, overproduction of uric acid occurs in a variety of metabolic derangements or medical disorders. Alternatively, hyperuricemia may be result of underexcretion of uric acid, such as conditions due to alterations in renal function. Hyperuricemia can lead to hyperuricosuria, which refers to excessive amounts of uric acid in the urine.

Numerous causes of hyperuricemia have been identified. Primary causes are innate to a subject and include genetic disorders such as hypoxanthine phosphoribosyltransferase deficiency and increased phosphoribosyl pyrophosphatase activity. Secondary causes are acquired disorders. These include hereditary fructose intolerance, glycogen storage disease, myeloproliferative disease, lymphoproliferative disease, hemolytic anemia, psoriasis, obesity, renal insufficiency, lead intoxication, chronic beryllium disease, sarcoidosis, and various drugs, e.g., low-dose salicylates, diuretics, pyrazinamide, ethambutol, nicotinamide and ethanol. Table 1 list various causes in terms of their pathophysiology.

TABLE 1
Causes of Hyperuricemia
Urate Overproduction

| | | |
|---|---|---|
| Primary idopathic | Myeloproliferative diseases | Rhabdomyolysis |
| HPRT deficiency | Polycythemia vera | Exercise |
| PRPP synthetase overactivity | Psoriasis | Alcohol |
| Hemolytic processes | Paget's disease | Obesity |
| Lymphoproliferative diseases | Glycogenosis III, V and VII | Purine-rich diet |

Decreased Uric Acid Excretion

| | | |
|---|---|---|
| Primary idiopathic | Starvation ketosis | Drug ingestion |
| Renal insufficiency | Berylliosis | Salicylates (>2 g/d) |
| Polycystic kidney disease | Sarcoidosis | Diuretics |
| Diabetes insipidus | Lead intoxication | Alcohol |
| Hypertension | Hypothyroidism | Levodopa |
| Acidosis | Hyperparathyroidism | Ethambutol |
| Lactic Acidosis | Toxemia of pregnancy | Pyrazinamide |
| Diabetic ketoacidosis | Bartter's syndrome | Nicotinic acid |
| | Down syndrome | Cyclosporine |

Combined Mechanisms

| | | |
|---|---|---|
| Glucose-6-phosphatase deficiency | Fructose-1-phosphate aldolase | Alcohol |
| | deficiency | Shock |

Certain events can cause hyperuricemia. Rapid purine degradation can cause hyperuricemia, e.g., in conditions of rapid cell proliferation or death, e.g., leukimic blast crises, cytotoxic cancer treatment, hemolysis or rhabdomylosis. Hyperuricemia can also result from excessive degradation of ATP from muscles, e.g., after exercise or due to glycogen storage diseases III, V and VII. Relatedly, hyperuricemia can be caused by myocardial infarction, smoke inhalation, and acute respiratory failure.

The methods and compositions of the present invention can be used to treat hyperuricemia related to most, if not all, of the above causes, e.g., by reducing serum uric acid levels.

(a) Disorders Associated with Hyperuricemia

Disorders associated with high levels of serum uric acid levels include, but are not limited to hyperuricemia, gout, urinary lithiasis, hyperuricemic nephropathy, acute uric acid nephropathy, cardiovascular disorders, renal disorders, metabolic disorders, fatty liver diseases, kidney stones and the like. Complications resulting from high levels of uric acid and uric acid crystal formation include, but are not limited to, muscle spasm, localized swelling, inflammation, joint pains, muscle fatigue, stress feelings, and myocardial infarction. The present invention provides compositions and methods for treating hyperuricemia and such related disorders.

Gout is a group of metabolic rheumatic disorders caused by aberrant purine metabolism and hyperuricemia and is the most common cause of an inflammatory arthropathy in middle-aged men. Gout is essentially a disorder of urate metabolism. Deposition of urate crystals in hyperuricemic individuals results in acute gout, characterized by agonizing pain and inflammation of rapid onset, most frequently affecting the first metatarsophalangeal joint. It can take decades for uric acid levels to rise to levels where uric acid crystals precipitate. Such precipitation can activate the NLRP3 (NALP3) inflammasome and result in a gouty attack. Hyperuricemia is associated with an increased risk of developing gout, and the risk of gout increases with the degree and duration of the hyperuricemia. Hyperuricemia in gout is typically accompanied by renal complications and suboptimal excretion of uric acid. Gouty attacks are typically severely painful and disabling.

A variety of risk factors have been identified for gout. In addition to hyperuricemia, these include obesity, diabetes, chronic kidney failure, hypertension, use of diuretic drugs, high purine diet, high fructose diet, exposure to lead, consumption of red meat and protein, and alcohol intake. See also Table 1. Gouty attack can be precipitated by perioperative ketosis in surgical patients, reduced body temperature, e.g., while sleeping, and by dehydration, e.g., by use of diuretic drugs. Genetic risk factors for gout and hyperuricemia have also been identified. Genetic deficiencies that can lead to increased serum urate levels include deficiency of the enzyme hypoxanthine-guaninephosphoribosyl transferase (HPRT), and overactivity of PPriboseP synthase (also known as PRPP (phosphoribosylpyrophosphate) synthetase). Inherited diseases including familial juvenile hyperuricemic nephropathy (FJHN) and autosomal-dominant medullary cystic kidney disease (ADMCKD) can also lead to reduced excretion of uric acid. Lesch-Nyhan syndrome (LNS), also known as Nyhan's syndrome or Kelley-Seegmiller syndrome, is a rare, inherited disorder caused by a deficiency of the enzyme hypoxanthine-guanine phosphoribosyltransferase (HGPRT), produced by mutations in the HPRT gene. LNS can lead to hyperuricemia, hyperuricosuria and corresponding gout and kidney problems. Polymorphisms in a variety of urate transporters have been identified as risk factors for hyperuricemia and gout. These urate transporters include SLC22A12 (URAT1), solute carrier family 2 (facilitative glucose transporter), member 9 gene (SLC2A9; Glut9), ABCG2 and SLC17A3. See, e.g., Cameron J S and Simmonds H A, Hereditary hyperuricemia and renal disease. Semin Nephrol. 2005 25:9-18; Bleyer A J and Hart T C, Genetic factors associated with gout and hyperuricemia. Adv Chronic Kidney Dis. 2006 13:124-30; Enomoto A., et al. Molecular identification of a renal urate anion exchanger that regulates blood urate levels. Nature 2002 417:447-52; Vitart V., et al. SLC2A9 is a newly identified urate transporter influencing serum urate concentration, urate excretion and gout. Nat Genet 2008 40:437-42; Dehghan A., et al. Association of three genetic loci with uric acid concentration and risk of gout: a genome-wide association study. Lancet 2008 372:1953-61.

Gout can be either acute or chronic. Triggers for acute gouty attacks include infection, intravenous contrast media, acidosis, and rapid fluctuations in serum uric acid concentrations such as with trauma, surgery, psoriasis flare-ups, initiation of chemotherapy, diuretic therapy, and stopping or starting allopurinol. Acute attacks usually begin in the joints of lower extremities. The attacks are characterized by joint pain and swelling. The first attack often comprises podagra, a sudden, unexplained swelling and pain of the big toe joint on just one foot. During an attack, which can last several days, pain can be so severe that patients are often unable to wear clothing or even touch bedsheets. Recurrent acute attacks can lead to chronic tophaceous deposits. Tophi are crystallized uric acid deposits that form firm swellings in joints, cartilage and bone. Tophi deposits sometimes disrupt the skin, exposing large chalky nodules. Extensive tophi can erode bone or other tissues and may require surgical removal.

In some patients, current medical treatments are ineffective at controlling serum uric acid levels at less than 6.0 mg/dL. Patients with such recalcitrant disease are deemed to have “refractory gout” and may exhibit severe clinical manifestations, including recurrent gout attacks, persistent swollen and painful joints, chronic pain, and progressive tophaceous deposits and joint damage. Refractory gout can have various causes, including but not limited to ineffectiveness of, or intolerance to, current treatments. Allopurinol remains the most frequent therapeutic for such patients, but it is only effective in some cases and additional therapies are often required. See, e.g., Fels and Sundy, Refractory gout: what is it and what to do about it? Cur Op. Rheum 2008, 20:198-202.

Patients having “severe gout” include those with serum uric acid levels that are greater than or equal to 8.0 mg/dL and have at least one gout tophus or gouty arthritis or have had at least three gouty flares in the past 18 months. Refractory gout comprises patients with severe gout wherein, in addition, conventional therapies are contraindicated or have been or become ineffective. For example, the patient may have a history of hypersensitivity or of failure to normalize serum uric acid (sUA) with at least 3 months of treatment with allopurinol at the maximum labeled dose (800 mg/dL in the U.S.), or at a medically appropriate lower dose based on dose-limiting toxicity or dose-limiting co-morbidity.

Medical complications arising from increased levels of serum uric acid are not limited to gout. High serum uric acid levels, even in the absence of uric acid crystal deposits, have been linked to a wide variety of cardiovascular and other conditions, including hypertension, metabolic syndrome, hyperlipidemia, insulin resistance, coronary artery disease, cerebrovascular disease, vascular dementia, preeclampsia, heart disease and kidney disease. High levels of uric acid can predict the onset of hypertension, obesity, diabetes and kidney disease. Risk factors that may contribute to hyperuricemia-related hypertension include those for risk of gout, e.g., dietary, environmental and genetic factors, as described herein. Such hypertension may further be related to chronic kidney disease and other renal diseases, e.g., microalbuminuria and renal dysfunction in subjects with normal renal function and impaired glomerular filtration rate in type 1 diabetics without proteinuria. Other renal problems associated with hyperuricemia include nephrolithiasis, urate nephropathy, and uric acid nephropathy. Nephrolithiasis, or kidney stones, are found most often in gout patients comprising uric acid stones, although hyperuricemia is also associated with other types of stones, e.g., calcium oxalate or calcium phosphate stones in non-gouty patients. Without being bound by theory, uric acid may act to seed calcium deposits. Urate nephropathy manifests from severe gout and is characterized by urate crystals in the renal interstitium. Uric acid nephropathy can cause renal failure from deposition of large amounts of crystals in the renal collecting ducts, pelvis and ureters. Other cardiovascular diseases associated with hyperuricemia include peripheral, carotid, and coronary vascular disease, stroke, preeclampsia, and vascular dementia. In some cases, drugs used to treat hyperuricemia, e.g., allopurinol, may be effective in treating diabetes, hypertension, and other uric acid related disorders. See, e.g., Feng et al., Uric Acid and Cardiovascular Risk, N Engl J Med 2008 359:1811-21.

Gout patients have higher death rates from all causes, although gout associated mortality is largely related to cardiovascular complications. In some cases, such cardiovascular complications relate to high serum uric acid levels, as described above. For example, hypertension is often observed in subjects with hyperuricemia. Higher serum uric acid levels are also associated with ongoing inflammatory response. For example, hyperuricemic patients often display higher levels of serum markers of inflammation, e.g., C reactive protein, fibrinogen, interleukin-6 (IL-6), and increased neutrophil count. The serum of gout patients contains high levels of inflammatory markers even in the absence of an ongoing gouty attack. And during attack, crystal formation activates monocytes and stimulates the release of inflammatory markers including tumor necrosis factor-α, IL-1, IL-6, IL-8, and cyclooxygenase-2. Ongoing low-grade inflammation among patients with gout may promote atherogenesis and thrombogenesis. Similar complications are also observed in other inflammatory rheumatic disorders associated with higher risk of cardiovascular disease, e.g., rheumatoid arthritis or systemic lupus erythematosus.

The present invention provides compositions and methods for treating hyperuricemia and such related disorders as described herein, including, but not limited to, gout, severe gout, refractory gout, chronic gout, cardiovascular disorders and related disorders, renal disorders and related disorders, fatty liver disease, kidney stones and aberrant metabolic conditions.

### (b) Diagnosis

One standard for diagnosis of gout comprises aspiration of tophi or synovial fluid from an inflamed joint. Tophi are crystallized uric acid deposits that form firm swellings in joints, cartilage and bone. Synovial fluid is a thick fluid which lubricates and cushions synovial joints, e.g., the wrist, elbow, knee, shoulder and hip joints. Needlelike monosodium urate crystals observed in the synovial fluid when viewed under a microscope are highly indicative of gout. However, aspiration techniques are not performed routinely in the clinical setting for various reasons, e.g., lack of availability of synovial fluid, time of the procedure and lack of physician experience. Alternately, clinical indications of disease, e.g., podagra, swollen and painful joints, tophi, and elevated serum urate can indicate hyperuricemia and gout. In some approaches, serum urate levels are measured during and following a gouty attack. Sometimes, elevated levels are only observed post-attack, e.g., two weeks later. In patients without hyperuricemia two weeks post-attack, e.g., uric acid levels below 4.0 mg/dL, gout is typically considered unlikely. Radiology can also assist in diagnosis of gout, e.g., to ascertain joint damage and urate deposits. Radiological techniques include x-ray film, computed tomography (CT) scans, magnetic resonance imaging (MRI), Dual Energy Computed Tomography (DECT), and ultrasound. See, e.g., Schlesinger, Diagnosis of Gout: Clinical, Laboratory, and Radiologic Findings, Am J Manag Care. 2005 11:S443-S450; Dore, The Gout Diagnosis, Cleve Clin J. Med. 2008 75:S17-S21. The aforementioned diagnostic techniques also may be used to monitor the efficacy of treatment.

Diagnosis of other conditions associated with an elevated serum uric acid level, for example, hyperuricemia, gout-associated inflammation, renal disorders, cardiovascular disease, aberrant metabolic conditions, fatty liver disease or kidney stones, may be performed according to current medical standards.

### II. Pharmaceutical Compositions

In some aspects, the present invention provides pharmaceutical compositions for treating a condition associated with an elevated serum uric acid level. Accordingly, in one embodiment a pharmaceutical composition comprises a compound of formula (I):

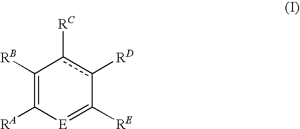

or a pharmaceutically acceptable salt thereof;
wherein
E is selected from N and CR^{N}; [symbol]
represents a single or double bond;
R^{A} is selected from H, C₁₋₄alkyl, OH, C₁₋₄alkoxy, halo, CO₂H and CO₂C₁₋₄alkyl;
R^{B} is selected from H, C₁₋₄alkyl, OH, C₁₋₄alkoxy, halo, or R^{A} and R^{B} together form an optionally substituted fused phenyl or heterocyclic ring;
R^{C} is selected from H, C₁₋₄alkyl, OH, C₁₋₄alkoxy and halo;
R^{D} is selected from H, C₁₋₄alkyl, C₂₋₄alkenyl, OH, C₁₋₄alkoxy, CO₂H, CO₂C₁₋₄alkyl and

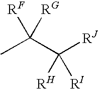

R^{E} is selected from C₁₋₄alkyl, OH, C₁₋₄alkoxy, halo, CO₂H, CO₂C₁₋₄alkyl, NH₂ and NHR^{K};
R^{N} is selected from H, C₁₋₄alkyl, OH and C₁₋₄alkoxy;
R^{F}, R^{G}, R^{H} and R^{I} are each independently H and C₁₋₄alkyl or R^{F} and R^{G} together form an oxo group or R^{F} and R^{H} form a bond;
R^{J} is selected from CH(CO₂H)NH₂, CH(CO₂C₁₋₄alkyl)NH₂, C(O)CO₂H, C(O)CO₂C₁₋₄alkyl, C(O)H, CO₂H, CO₂C₁₋₄alkyl, C(O)NH₂, C(O)NHR^{L}, CH₂NH₂, CH₂NHC₁₋₄alkyl and CH₂N(C₁₋₄alkyl)₂;
R^{K} is selected from H, C₁₋₄alkyl and C(O)H; and
R^{L} is selected from H, C₁₋₄alkyl and optionally substituted phenyl or heterocyclic ring, wherein optionally substituted phenyl or heterocyclic ring is optionally substituted with one or more, C₁₋₄alkyl, OH, C₁₋₄ alkoxy, CO₂H, tetrazole, CO₂C₁₋₄alkyl, halo, NH₂, NHC₁₋₄alkyl, N(C₁₋₄alkyl)₂,

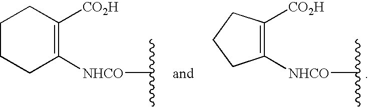

In another embodiment, a pharmaceutical composition for use in methods of the invention comprises a compound of formula (II):

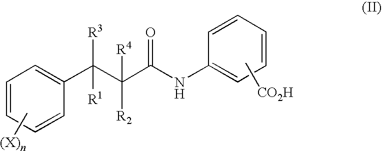

or a pharmaceutically acceptable salt thereof,
wherein each of R¹ and R² is independently selected from a hydrogen atom or a C₁-C₄ alkyl group, R³ and R⁴ are each hydrogen atoms or together form another chemical bond, each X is independently selected from a hydroxyl group, a halogen atom, a C₁-C₄ alkyl group or a C₁-C₄ alkoxy group, or when two X groups are alkyl or alkoxy groups, they may be connected together to form a ring, and n is an integer from 1 to 3.

The carboxyl group may be in the 2-, 3- or 4-position of the aromatic ring. In one embodiment the carboxyl group is in the 2-position.

In one embodiment at least one of R¹ and R² is a hydrogen atom. In one such embodiment, both of R¹ and R² are hydrogen atoms.

In one embodiment R³ and R⁴ taken together form a chemical bond. Such compounds having an unsaturated bond may be in the form of E or Z geometric isomers.

In one embodiment n is 1 or 2 and each X, which may be the same or different, is selected from halogen, C₁-C₄ alkyl or C₁-C₄ alkoxy. In one such embodiment X is selected from halogen and C₁-C₄ alkoxy. In one embodiment, n is 2 and both X are selected from C₁-C₄ alkoxy. In one such embodiment both X are methoxy.

In one embodiment compounds useful in the present invention are those of formula (III):

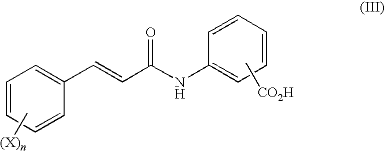

and pharmaceutically acceptable salts thereof.

Examples of compounds of formula (III) include:
2-[[3-(2-methylphenyl)-1-oxo-2-propenyl]amino]benzoic acid;
2-[[3-(3-methylphenyl)-1-oxo-2-propenyl]amino]benzoic acid;
2-[[3-(4-methylphenyl)-1-oxo-2-propenyl]amino]benzoic acid;
2-[[3-(2-ethylphenyl)-1-oxo-2-propenyl]amino]benzoic acid;
2-[[3-(3-ethylphenyl)-1-oxo-2-propenyl]amino]benzoic acid;
2-[[3-(4-ethylphenyl)-1-oxo-2-propenyl]amino]benzoic acid;
2-[[3-(2-propylphenyl)-1-oxo-2-propenyl]amino]benzoic acid;
2-[[3-(3-propylphenyl)-1-oxo-2-propenyl]amino]benzoic acid;
2-[[3-(4-propylphenyl)-1-oxo-2-propenyl]amino]benzoic acid;
2-[[3-(2-hydroxyphenyl)-1-oxo-2-propenyl]amino]benzoic acid;
2-[[3-(3-hydroxyphenyl)-1-oxo-2-propenyl]amino]benzoic acid;
2-[[3-(4-hydroxyphenyl)-1-oxo-2-propenyl]amino]benzoic acid;
2-[[3-(2-chlorophenyl)-1-oxo-2-propenyl]amino]benzoic acid;
2-[[3-(3-chlorophenyl)-1-oxo-2-propenyl]amino]benzoic acid;
2-[[3-(4-chlorophenyl)-1-oxo-2-propenyl]amino]benzoic acid;
2-[[3-(2-fluorophenyl)-1-oxo-2-propenyl]amino]benzoic acid;
2-[[3-(3-fluorophenyl)-1-oxo-2-propenyl]amino]benzoic acid;
2-[[3-(4-fluorophenyl)-1-oxo-2-propenyl]amino]benzoic acid;
2-[[3-(2-bromophenyl)-1-oxo-2-propenyl]amino]benzoic acid;
2-[[3-(3-bromophenyl)-1-oxo-2-propenyl]amino]benzoic acid;
2-[[3-(4-bromophenyl)-1-oxo-2-propenyl]amino]benzoic acid;
2-[[3-(2,3-dimethoxyphenyl)-1-oxo-2-propenyl]amino]benzoic acid;
2-[[3-(3,4-dimethoxyphenyl)-1-oxo-2-propenyl]amino]benzoic acid;
2-[[3-(2,4-dimethoxyphenyl)-1-oxo-2-propenyl]amino]benzoic acid;
2-[[3-(2,3-dimethylphenyl)-1-oxo-2-propenyl]amino]benzoic acid;
2-[[3-(3,4-dimethylphenyl)-1-oxo-2-propenyl]amino]benzoic acid;
2-[[3-(2,4-dimethylphenyl)-1-oxo-2-propenyl]amino]benzoic acid;
2-[[3-(2,3-diethoxyphenyl)-1-oxo-2-propenyl]amino]benzoic acid;
2-[[3-(3,4-diethoxyphenyl)-1-oxo-2-propenyl]amino]benzoic acid;
2-[[3-(2,4-diethoxyphenyl)-1-oxo-2-propenyl]amino]benzoic acid;
2-[[3-(2,3-dipropoxyphenyl)-1-oxo-2-propenyl]amino]benzoic acid;
2-[[3-(3,4-dipropoxyphenyl)-1-oxo-2-propenyl]amino]benzoic acid;
2-[[3-(2,4-dipropoxyphenyl)-1-oxo-2-propenyl]amino]benzoic acid;
2-[[3-(2,3-diethylphenyl)-1-oxo-2-propenyl]amino]benzoic acid;
2-[[3-(3,4-diethylphenyl)-1-oxo-2-propenyl]amino]benzoic acid;
2-[[3-(2,4-diethylphenyl)-1-oxo-2-propenyl]amino]benzoic acid;
2-[[3-(2,3-dipropylphenyl)-1-oxo-2-propenyl]amino]benzoic acid;
2-[[3-(3,4-dipropylphenyl)-1-oxo-2-propenyl]amino]benzoic acid;
2-[[3-(2,4-dipropylphenyl)-1-oxo-2-propenyl]amino]benzoic acid;
2-[[3-(2-methoxy-3-methylphenyl)-1-oxo-2-propenyl]amino]benzoic acid,
2-[[3-(3-methoxy-4-methylphenyl)-1-oxo-2-propenyl]amino]benzoic acid;
2-[[3-(2-methoxy-3-methylphenyl)-1-oxo-2-propenyl]amino]benzoic acid;
2-[[3-(2-methoxy-4-methylphenyl)-1-oxo-2-propenyl]amino]benzoic acid;
2-[[3-(2-methoxy-3-chlorophenyl)-1-oxo-2-propenyl]amino]benzoic acid;
2-[[3-(3-methoxy-4-chlorophenyl)-1-oxo-2-propenyl]amino]benzoic acid;
2-[[3-(2-methoxy-3-chlorophenyl)-1-oxo-2-propenyl]amino]benzoic acid;
2-[[3-(2-methoxy-4-chlorophenyl)-1-oxo-2-propenyl]amino]benzoic acid;
2-[[3-(2-methoxy-3-hydroxyphenyl)-1-oxo-2-propenyl]amino]benzoic acid;
2-[[3-(3-methoxy-4-hydroxyphenyl)-1-oxo-2-propenyl]amino]benzoic acid;
2-[[3-(2-methoxy-3-hydroxyphenyl)-1-oxo-2-propenyl]amino]benzoic acid;
2-[[3-(2-methoxy-4-hydroxyphenyl)-1-oxo-2-propenyl]amino]benzoic acid;
2-[[3-(3,4-trimethylenephenyl)-1-oxo-2-propenyl]amino]benzoic acid;
2-[[3-(2,3-trimethylenephenyl)-1-oxo-2-propenyl]amino]benzoic acid;
2-[[3-(3,4-methylenedioxyphenyl)-1-oxo-2-propenyl]amino]benzoic acid; and
2-[[3-(3,4-ethylenedioxyphenyl)-1-oxo-2-propenyl]amino]benzoic acid.

In one embodiment the compound of formula (III) is Tranilast (TNL), which is 2-[[3-(3,4-dimethoxyphenyl)-1-oxo-2-propenyl]amino]benzoic acid. In other embodiments the compound is 3-hydroxykynurenic acid (3-HKA), 3-hydroxyanthranilic acid (3-HAA), picolinic acid (PA), or quinolinic acid (QA).

As used herein the term “treating” includes treating, preventing and/or ameliorating the condition or disease to which it refers.

As used herein the term “elevated serum uric acid level” means serum uric acid level greater than normal. In some instances, elevated serum uric acid levels are above the mean level in a given population, such as gender and/or age.

As used herein the term “hyperuricemia” means serum uric acid level greater than the normal level for the population. In some instances, hyperuricemia includes serum uric acid levels greater than or equal to 6.0 mg/dL, 6.8 mg/dL, 7 mg/dL or 8 mg/dL.

As used herein the term “severe gout” includes gout present in a subject having serum uric acid levels that are greater than or equal to 8.0 mg/dL and have at least one gout tophus or gouty arthritis or have had at least three gouty flares in the past 18 months.

As used herein the term “chronic gout” includes gout present in a subject having recurrent or prolonged gout flares, tophus formation, chronic inflammatory arthritis and/or joint destruction associated with gout.

As used herein the term “acute gout” includes gout present in a subject that has had or is having at least one gouty symptom, such as a gout flare or gouty attack.

As used herein the term “gout-associated inflammation” refers to local or systemic inflammation due to immune response to urate crystals.

As used herein, the term “C₁-C₄ alkyl” refers to linear or branched hydrocarbon chains having 1 to 4 carbon atoms. Examples of such groups include methyl, ethyl, n-propyl, isopropyl, n-butyl, sec-butyl and tert-butyl.

As used herein the term “C₂-C₄ alkenyl” refers to linear or branched hydrocarbon chains having 2 to 4 carbon atoms and one or two double bonds. Examples of such groups include vinyl, propenyl, butenyl and butadienyl.

As used herein, the term “C₁-C₄ alkoxy” refers to hydroxy groups substituted with linear or branched alkyl groups having 1 to 4 carbon atoms. Examples of such groups include methoxy, ethoxy, n-propoxy, isopropoxy, n-butoxy, sec-butoxy and tert-butoxy.

In some embodiments, the term “C₁-C₄ alkyl” refers to linear, branched, or cyclized hydrocarbon chains having 1 to 6 carbon atoms. In some embodiments, the term “C₁-C₄ alkoxy” refers to hydroxy groups substituted with linear or branched alkyl groups having 1 to 6 carbon atoms. Examples of such cyclized groups include cyclopropyl, cyclopropyl methyl, cyclobutyl, cyclopentyl, or cyclohexyl and related alkoxy groups.

As used herein, the term “halogen” or “halo” refers to fluoro, chloro or bromo atoms.

As used herein the term “heterocyclic ring” refers to optionally substituted unsaturated, five- to six-membered cyclic structure in which one or more skeletal atoms is oxygen, nitrogen, sulfur, or combinations thereof. Heterocyclic ring, includes, but is not limited to furanyl, imidazolyl, isoxazolyl, isothiazolyl, oxadiazolyl, oxazolyl, purinyl, pyrazinyl, pyrazolyl, pyridinyl, pyrimidinyl, pyrrolyl, triazolyl, thiazolyl, thiophenyl, tetrazolyl, thiadiazolyl, and thienyl.

Suitable pharmaceutically acceptable salts include, but are not limited to, salts of pharmaceutically acceptable inorganic acids such as hydrochloric, sulphuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, maleic, citric, lactic, mucic, gluconic, benzoic, succinic, oxalic, phenylacetic, methanesulphonic, toluenesulphonic, benezenesulphonic, salicyclic sulphanilic, aspartic, glutamic, edetic, stearic, palmitic, oleic, lauric, pantothenic, tannic, ascorbic and valeric acids.

Base salts include, but are not limited to, those formed with pharmaceutically acceptable cations, such as sodium, potassium, lithium, calcium, magnesium, ammonium and alkylammonium.

Basic nitrogen-containing groups may be quarternised with such agents as lower alkyl halide, such as methyl, ethyl, propyl, and butyl chlorides, bromides and iodides; dialkyl sulfates like dimethyl and diethyl sulfate; and others.

Compounds of formula (I) and their pharmaceutically acceptable salts are known and may be prepared by methods known in the art, see U.S. Pat. No. 3,940,422, the contents of which are incorporated herein by reference.

It will also be recognized that some compounds of formula (I) may possess asymmetric centers and are therefore capable of existing in more than one stereoisomeric form. The invention thus also relates to compounds in substantially pure isomeric form at one or more asymmetric centers, e.g., greater than about 90% ee, such as about 95% or 97% ee or greater than 99% ee, as well as mixtures, including racemic mixtures, thereof. Such isomers may be prepared by asymmetric synthesis, for example using chiral intermediates, or by chiral resolution. Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention.

Unless otherwise stated, compounds depicted herein that contain a double bond are also meant to include both cis/trans (or E/Z) isomeric forms and both isomeric forms are within the scope of the invention. Once such compound containing a double bond is referred to as N-[3,4-dimethoxycinnamoyl]-anthranilic acid or 2-[[3-(3,4-dimethoxyphenyl)-1-oxo-2-propenyl]amino]benzoic acid and may also be referred to as Tranilast.

Unless otherwise stated, structures depicted herein are also meant to include compounds which differ only in the presence of one or more isotopically enriched atoms. For example, compounds represented by the present structures, but with the replacement of a hydrogen by a deuterium or tritium, or the replacement of a carbon by a ¹³C- or ¹⁴C-enriched carbon are within the scope of this invention.

There are a number of synthetic pathways which yield a deuterated analog of tranilast. Scheme 1 describes but one method to prepare such a deuterated analog; other methods are well-known to those of skill in the art. Following a standard amide synthesis, such as that shown below, the starting material, deuterated anthranilic acid, A-1 (CAS 60124-83-6), can be reacted with a cinnamic acid analog, B-1, to yield a deuterated analog of tranilast, C-1.

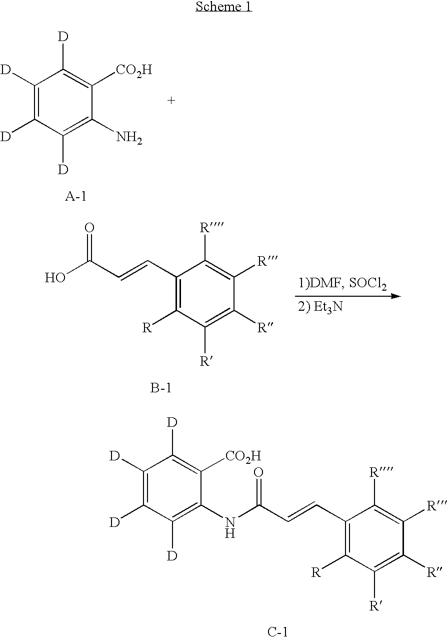

Alternately, a deuterated cinnamic acid analog, B-2, can be prepared from a deuterated dimethoxybenzaldehyde derivative, such as:

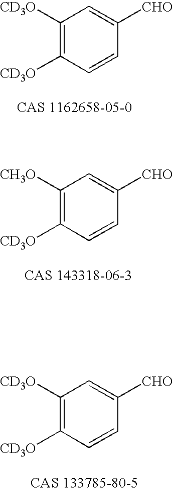

([CAS 1162658-05-0] See Zou et al. Chemistry Express 1991 6(3):213-216 “Synthesis of 1,2,4-trimethoxy benzene and its three monomethoxy-d₃ derivatives;” [CAS 143318-06-3 and CAS 1337-80-5] see US 2009/0062300 to Czarnik)

A deuterated dimethoxybenzaldehyde derivative can be converted to a deuterated cinnamic acid analog according to Scheme 2, where each of R^{a} and R^{b} is independently —CH₃ or —CD₃.

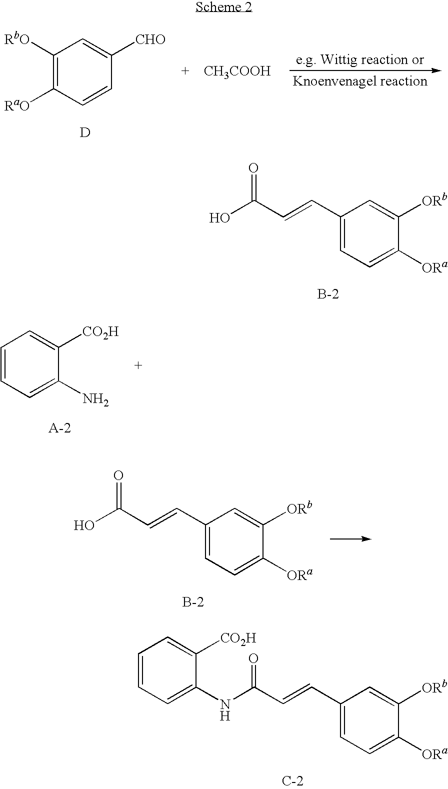

### Example 1

### Synthesis of Deuterated Tranilast (C-1)

To a solution of a cinnamic acid analog (B-1) (1 equiv.) in anhydrous DCM and catalytic DMF, thionyl chloride (1.1 equiv.) is added at 0-5° C. The reaction is refluxed for 1 h and evaporated under reduced pressure. The residue is triturated with DCM and evaporated. The acid chloride is then dissolved in DCM and added to a solution of deuterated anthranilic acid (A-1, C/D/N Isotopes (Pointe-Claire, Quebec Canada)) (0.9 equiv.) and triethylamine (2-4 equiv.) in DCM at 0-5° C. The reaction is monitored by TLC and product is isolated after washing the reaction mixture with saturated aq. NaCl solution (X3), is dried over anhydrous Na₂SO₄ and is evaporated. The crude product (C-1) is purified by column chromatography.

### Example 2

### Synthesis of Deuterated Tranilast (C-2)

### Example 2A

### Synthesis of Deuterated Cinnamic Acid Analog (B-2)

Deuterated cinnamic acid analog (B-2) is prepared by the Doebner modification of the Knoevenagel condensation of deuterated dimethoxybenzaldehyde derivative (D) and malonic acid in pyridine. The reaction is carried out as described for the synthesis of 2,3-dimethoxycinnamic acid in Organic Synthesis, Collected Vol. 4, pp 327-329. D (0.01 mol) and malonic acid (0.02 mol) in pyridine (10 mL) are heated and when the malonic acid is dissolved, piperidine (0.2 mL) is added. The reaction is heated as described in the above reference and worked up using conditions as described to afford B-2.

### Example 2B

### Synthesis of Deuterated Tranilast C-2

To a solution of a cinnamic acid analog (B-2) (1 equiv.) in anhydrous DCM and catalytic DMF, thionyl chloride (1.1 equiv.) is added at 0-5° C. The reaction is refluxed for 1 h and evaporated under reduced pressure. The residue is triturated with DCM and evaporated. The acid chloride is then dissolved in DCM and added to a solution of anthranilic acid (A-2) (0.9 equiv.) and triethylamine (2-4 equiv.) in DCM at 0-5° C. The reaction is monitored by TLC and product is isolated after washing the reaction mixture with saturated aq. NaCl solution (X3), is dried over anhydrous Na₂SO₄ and is evaporated. The crude product (C-2) is purified by column chromatography.

Without limiting the present invention to any one theory or mode of action, the compounds of formula (I) can be orally active anti-hyperuricemic compounds, and can have a direct or indirect affect on uric acid transporters in the brush border membrane. A particularly preferred compound of the invention is known either of the chemical names N-[3,4-dimethoxycinnamoyl]-anthranilic acid or 2-[[3-(3,4-dimethoxyphenyl)-1-oxo-2-propenyl]amino]benzoic acid and may also be referred to as Tranilast. The structure is depicted below:

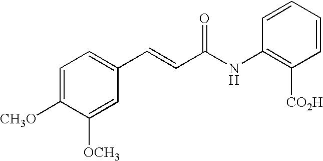

Anthranilic acids, such as N-[3,4-dimethoxycinnamoyl]-anthranilic acid (also known by benzoic acid names such as 2-[[3-(3,4-dimethoxyphenyl)-1-oxo-2-propenyl]amino]benzoic acid are useful for the methods of the present invention. In some embodiments, esters and amides of anthranilic acids are used in the methods of the present invention. In some embodiments, bioisosteric replacements known in the art for carboxylic acids, esters and amides are used.

The compounds of formula (I) or formula (II) or pharmaceutically acceptable salts thereof or their antagonists may be linked, bound or otherwise associated with any proteinaceous or non-proteinaceous molecules. For example, in one embodiment of the present invention said compounds of formula (I) or pharmaceutically acceptable salts thereof may be associated with a molecule which permits targeting to a localized region.

Metabolites and derivatives of compounds of formula (I) and formula (II), including Tranilast and pharmaceutically acceptable salts thereof, are contemplated for use herein with another therapy or treatment regime. In some embodiments, the use of Tranilast and a second drug or agent can allow the use of a lower dose of Tranilast or the second drug or agent than would ordinarily be used.

In some embodiments, the compounds of the present invention may be modified in order to reduce side effects, improve pharmacokinetic and/or pharmacodynamic profiles. Incorporation of a heavy atom particularly substitution of deuterium for hydrogen, can give rise to an isotope effect that can alter the pharmacokinetics of the drug. The safety profile of a composition may be improved through incorporation of a heavy atom (e.g., deuterium). For example, compositions with substituted deuterium may be delivered in smaller doses with equivalent efficacy. By reducing the dosage, corresponding side effects may be diminished as well.

Replacement within a drug with a heavy isotope can alter its physicochemical properties such as pKa and lipid solubility. These changes may influence the fate of the drug at different steps along its passage through the body. Absorption, distribution, metabolism or excretion can be changed. Absorption and distribution are processes that depend primarily on the molecular size and the lipophilicity of the substance.

Drug metabolism can give rise to large isotopic effect if the breaking of a chemical bond to a deuterium atom is the rate limiting step in the process. While some of the physical properties of a stable isotope-labeled molecule are different from those of the unlabeled one, the chemical and biological properties are the same, with one important exception: because of the increased mass of the heavy isotope, any bond involving the heavy isotope and another atom will be stronger than the same bond between the light isotope and that atom. In any reaction in which the breaking of this bond is the rate limiting step, the reaction will proceed slower for the molecule with the heavy isotope due to kinetic isotope effect. A reaction involving breaking a C-D bond can be up to 700 percent slower than a similar reaction involving breaking a C-H bond.

More caution has to be observed when using deuterium labeled drugs. If the C-D bond is not involved in any of the steps leading to the metabolite, there may not be any effect to alter the behavior of the drug. If a deuterium is placed at a site involved in the metabolism of a drug, an isotope effect will be observed only if breaking of the C-D bond is the rate limiting step. There are evidences to suggest that whenever cleavage of an aliphatic C-H bond occurs, usually by oxidation catalyzed by a mixed-function oxidase, replacement of the hydrogen by deuterium will lead to observable isotope effect. It is also important to understand that the incorporation of deuterium at the site of metabolism slows its rate to the point where another metabolite produced by attack at a carbon atom not substituted by deuterium becomes the major pathway by a process called “metabolic switching.”

For example, substitution of hydrogens with heavier isotopes such as deuterium, i.e., ²H, can afford certain therapeutic advantages resulting from greater metabolic stability, for example increased in vivo half-life or reduced dosage requirements and, hence, may be preferred in some circumstances.

Other related compounds that can be used within the scope of the present invention include those disclosed by PCT Patent Applications: WO2008131481 to Kelly and Gilbert, filed Apr. 24, 2008 and entitled “Treatment of Mesangioproliferative Diseases,” WO2008003141 to Williams et al., filed Jul. 5, 2007 and entitled “Therapeutic Agents,” WO2006087393 (EP1856030) to Relivia and Ghisalberti, filed Feb. 21, 2006 and entitled “Structural Analogues of Avenanthramides, Their Use in Compositions Useful in the Treatment of Dermatological Disorders,” WO2007044724 (EP1940792) to Kearney et al., filed Oct. 5, 2006 and entitled “Aminopyrimidine, Aminopyridine and Aniline Derivatives Inhibitors of PIM-I and/or PIM-3,” WO2006117602 to Bassoli et al., filed Apr. 12, 2006 and entitled “Use of Amide Derivatives as Taste-Modifying Agents, Flavouring Compositions and Products Containing Them,” and WO2006134013 to Vielhaber and Schmaus, filed SS and entitled “Mixtures Comprising Anthranilic Acid Amides and Cooling Agents as Cosmetic and Pharmaceutical Compositions for Alleviating Itching”; U.S. Pat. No. 7,429,593 to Yamamori et al., filed Sep. 14, 2001 and entitled “Utilities of Amide Compounds”; U.S. Patent Publication 20090197825 to Quart et al., filed Nov. 26, 2008 and entitled “Novel Compounds and Compositions and Methods of Use”; U.S. Patent Publication 20060089413 to Schmaus et al., filed Nov. 20, 2003 and entitled “Anthranilic Acid Amides and Derivatives Thereof as Cosmetic and Pharmaceutical Agents”; and European Patent Publication EP0227431 to Outred, filed Dec. 17, 1986 and entitled “Heterocyclic Amides.”

### III. Combination Treatments

In some embodiments, the compounds of the present invention are used in combination with other therapeutic agents to bring about a desired effect. Selection of additional agents depends, in large part, on the desired target therapy. It is known that modulation of serum uric acid levels can be further improved by the addition of a second agent to the therapeutic regimen. See, e.g., Chung et al., Optimizing therapy with allopurinol: factors limiting hypouricemic efficacy. Am J Med Sci. 2008 March; 335(3):219-26. Combination therapy includes administration of a single pharmaceutical dosage formulation that contains a compound of the present invention, e.g., Tranilast, as well as administration of a compound of the present invention and each additional active agent in its own separate pharmaceutical dosage formulation. For example, a compound of formula (II) (e.g., Tranilast), or a pharmaceutically acceptable salt thereof, and a xanthane oxidase inhibitor (e.g., allopurinol or febuxostat), can be administered to the human subject together in a single oral dosage composition, such as a tablet or capsule, or each agent can be administered in separate oral dosage formulations. Where separate dosage formulations are used, the compositions of the present invention and one or more additional active agents can be administered at essentially the same time (i.e., simultaneously or concurrently), or at separately staggered times (i.e., sequentially). Combinations can include two or more active agents in addition to one or more compounds of the present invention. Combination therapy is understood to include all these regimens.

### (a) Hyperuricemia and Gout

Hyperuricemia and chronic gout are treated with agents that lower urate levels, e.g., xanthine oxidase inhibitors and uricosuric agents, thereby reducing uric acid levels and potential crystal formation. Xanthine oxidase is involved in purine metabolism and inhibiting the enzyme reduces uric acid levels. Allopurinol, a xanthine oxidase inhibitor, is the current first line standard of care for lowering urate levels. Another xanthine oxidase inhibitor, Febuxostat, was approved for treatment of gout in February 2009. Other xanthine oxidase inhibitors include oxypurinol, tisopurine, inositols (e.g., phytic acid and myo-inositol) and potentially propolis. Uricosuric agents enhance excretion of uric acid and generally act by lowering the absorption of uric acid from the kidneys back to the blood, e.g., by inhibiting urate transporters, e.g, SLC22A12. Probenecid is the most commonly used uricosuric agent in the U.S. and may be given in combination with allopurinol to refractory gout patients. Benzbromarone and sulfinpyrazone are also used as first line uricosuric agents. Guaifenesin, losartan, atorvastatin, amlodipine, adrenocorticotropic hormone (ACTH or corticotropin), fenofibrate and cortisone also have uricosuric effects. Additionally, other uricosuric agents are being developed or are in clinical trials, such uricase enzymes including rasburicase or the pegylated uricase enzyme PURICASE® (Pegloticase), which has completed Phase III trials. Uricase or urate oxidase enzymes can lower uric acid levels by converting uric acid into allantoin, a benign end metabolite which is easily excreted in the urine. IL-6 has also been shown to reduce serum uric acid levels and proposed as a treatment for hyperuricemia and gout. See U.S. Pat. No. 6,007,804, issued Dec. 28, 1999 and entitled “IL-6 as serum uric acid decreasing compound.” Interleukin 1 (IL-1) antagonists are being developed for chronic gout. For example, Canakinumab (ACZ885) is a human monoclonal antibody targeted at IL-1 beta, being developed by Novartis for the treatment of rheumatoid arthritis and gout. Rilonacept, marketed under the trade name Arcalyst by Regeneron Pharmaceuticals, is under going trials is a dimeric fusion protein and IL-1 blocker, also undergoing trials as a treatment for gout. See also U.S. Patent Publication No. 2008/0300185, filed Oct. 19, 2007 and entitled “Use of IL-1 antagonists to treat gout and pseudogout.” Diet and lifestyle can be modified to reduce urate levels, e.g., lowering red meat or alcohol consumption, or substituting alternate treatments for diuretic drugs.

Acute gout typically presents with inflammation, pain and swelling. Urate-lowering therapies, described above for hyperuricemia and chronic gout, are usually not used until the acute phase of gout has resolved because fluctuations in serum uric acid can exacerbate the inflammatory process. Therapy is generally directed at reducing the inflammation, pain and swelling, e.g., anti-inflammatory agents and pain killers. Nonsteroidal anti-inflammatory drugs (NSAIDs) or corticosteroids are typically given for acute gouty attacks, depending on co-morbidities. NSAIDS include, but are not limited to, indomethacin, naproxen and sulindac. Corticosteroids include, but are not limited to, prednisone or methylprednisone. Colchicine is given as a second line therapy, but has toxicities at higher levels. For example, Colchicine can lead to bone marrow suppression and neuromyopathy in patients with severe renal or hepatic impairment. More common adverse effects include nausea, vomiting, and diarrhea. In some cases, opioid agents are given to acute gout sufferers.

The compositions of the invention can be used in combination with one or more agents described herein for reducing uric acid or otherwise treating gout. When a combination use is desired, the composition of the invention and the one or more gout treatments can be administered or applied sequentially or simultaneously. By way of example only, the composition of the invention may further comprise one or more uricosuric agents in a single dosage form. Alternatively, the composition of the invention and the one or more uricosuric agents are formulated as separate dosage forms and administrated or applied simultaneously or sequentially to a subject.

In some embodiments of the invention, methods and compositions of the invention are administered before, concurrent to or after treatment of a subject in need thereof with one or more standards of care used to treat gout. Examples of Standards of Care for gout include but are not limited to administration of one or more therapeutic agents to treat pain and reduce urate in blood. For example, drugs used to lower the amount of urate or analgesic drugs may be administered before, concurrent to or after treatment of a subject in need thereof with compounds and methods of the present invention.

In some embodiments, pharmacologically active compounds, e.g., for the treatment of inflammation, pain, or hyperuricemia, are administered before, concurrent to or after treatment of a subject in need thereof with compounds and methods of the present invention. Such pharmacologically active compounds include, without limitation, the following:

Xanthane oxidase inhibitors. In one embodiment, a compound of the invention, e.g., Tranilast, is administered before, concurrently or subsequent to administration of a xanthane oxidase inhibitor. Xanthane oxidase inhibitors lower the amount of urate in blood. Xanthane oxidase inhibitors include, but are not limited to, allopurinol, febuxostat, oxypurinol, tisopurine, or an inositol. In one embodiment, a compound of the invention is administered before, concurrently or subsequent to administration of allopurinol. In one embodiment, a compound of the invention is administered before, concurrently or subsequent to administration of Febuxostat.

Uricosuric agents. In one embodiment, a compound of the invention is administered before, concurrently or subsequent to administration of a uricosuric agent. Uricosuric agents enhance the excretion of uric acid. Uricosuric agents include, but are not limited to, probenecid, benzbromarone, sulfinpyrazone, guaifenesin, losartan, atorvastatin, amlodipine, adrenocorticotropic hormone (ACTH or corticotropin), or fenofibrate. In one embodiment, a compound of the invention is administered before, concurrently or subsequent to administration of probenecid, benzbromarone, or sulfinpyrazone.

Another uricosuric agent is Ardea Biosciences' RDEA594, a metabolite of their antiviral RDEA806. RDEA594 is believed to be responsible for essentially all of the uric acid lowering effects seen with RDEA806. The chemical structure of RDEA806 may be represented as follows:

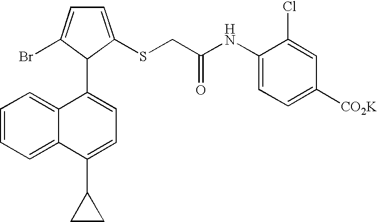

The chemical structure of RDEA594 is believed to be as follows:

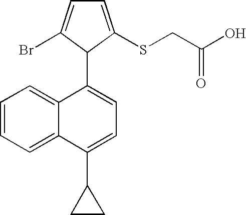

Still another Ardea Biosciences uricosuric agent is RDEA684, which is currently in preclinical development.

Uricase enzymes. In one embodiment, a compound of the invention is administered before, concurrently or subsequent to administration of a uricase enzyme. Uricase or urate oxidase enzymes are found in many mammals but not humans. They can lower uric acid levels by converting uric acid into allantoin, a benign end metabolite which is easily excreted in the urine. Uricase enzymes include, but are not limited to, rasburicase or a pegylated uricase enzyme (PEG-uricase). In some embodiments, the pegylated uricase enzyme is PURICASE® (Pegloticase).

Non-steroidal anti-inflammatory drugs (NSAIDs). In one embodiment, a compound of the invention is administered before, concurrently or subsequent to administration of one or more non-steroidal anti-inflammatory drugs (NSAIDs). For example, administration of an NSAID can reduce the pain and inflammation experienced with gout. A non-limiting list of NSAIDs includes diclofenac, indomethacin, naproxen, sulindac and lumiracoxib. Further NSAIDs capable of use with methods and compositions of the invention are disclosed in U.S. Pat. Nos. 7,423,042; 7,341,737; 7,303,761; and 6,787,155, all of which are hereby incorporated by reference in their entirety.

Cox-2 selective inhibitors. In one embodiment, a compound of the invention is administered before, concurrently or subsequent to administration of one or more Cox-2 inhibitors. Cox-2 inhibitors are a newer type of NSAID which are designed to be less harmful to the stomach. Etoricoxib is the Cox-2 selective inhibitor normally prescribed to treat gout. COX-2 inhibitors have been reported in the art and many chemical structures are known to produce inhibition of cyclooxygenase-2. COX-2 inhibitors are described, for example, in U.S. Pat. Nos. 5,616,601; 5,604,260; 5,593,994; 5,550,142; 5,536,752; 5,521,213; 5,474,995; 5,639,780; 5,604,253; 5,552,422; 5,510,368; 5,436,265; 5,409,944; and 5,130,311, all of which are hereby incorporated by reference in their entirety. Certain preferred COX-2 inhibitors include celecoxib (SC-58635), 5-bromo-2-(4-fluorophenyl)-3-(4-(methylsulfonyl)phenyl)-thiophene (DUP-697), flosulide (CGP-28238), meloxicam, 6-methoxy-2 naphthylacetic acid (6-MNA), MK-966 (also known as Vioxx), nabumetone (prodrug for 6-MNA), nimesulide, N-[2-(cyclohexyloxy)-4-nitrophenyl]-methanesulfonamide (NS-398), SC-5766, SC-58215, or 3-Formylamino-7-methylsulfonylamino-6-phenoxy-4H-1-benzopyran-1-one (T-614); or combinations thereof.

Corticosteroids. In one embodiment, a compound of the invention is administered before, concurrently or subsequent to administration of one or more corticosteroids. Corticosteroids are a type of steroid, and they sometimes are used in severe cases of gout. In some cases, a corticosteroid can be injected directly into the affected joint. The type of corticosteroid received will depend on the size of the affected joint. Suitable corticosteroids which may be used in combination with the compounds of the invention include, but are not limited to, methyl prednisolone, prednisolone, dexamethasone, fluticasone propionate, 6α,9α-difluoro-17-[(2-furanylcarbonyl)oxy]-11β-hydroxy-16α-methyl-3-oxo-androsta-1,4-diene-17β-carbothioic acid S-fluoromethyl ester, 6α,9α-difluoro-11β-hydroxy-16α-methyl-3-oxo-17.alpha.-propionyloxy-androsta-1,4-diene-17β-carbothioic acid S-(2-oxo-tetrahydro-furan-3S-yl) ester, beclomethasone esters (e.g., the 17-propionate ester or the 17,21-dipropionate ester), budesonide, flunisolide, mometasone esters (e.g., the furoate ester), triamcinolone acetonide, rofleponide, ciclesonide, butixocort propionate, RPR-106541, and ST-126. Preferred corticosteroids include fluticasone propionate, 6α,9α-difluoro-11β-hydroxy-16α-methyl-17α-[(-4-methyl-1,3-thiazole-5-carbonyl)oxy]-3-oxo-androsta-1,4-diene-17β-carbothioic acid S-fluoromethyl ester and 6α,9α-difluoro-17α-[(2-furanylcarbonyl)oxy]-11β-hydroxy-16α-methyl-3-oxo-androsta-1,4-diene-17β-carbothioic acid S-fluoromethyl ester, more preferably 6α,9α-difluoro-17α-[(2-furanylcarbonyl)oxy]-11β-hydroxy-16α-methyl-3-oxo-androsta-1,4-diene-17β-carbothioic acid S-fluoromethyl ester; and those disclosed in U.S. Pat. Nos. 7,288,536; 7,291,609; 7,157,433; 7,091,187; and 6,897,206, which are incorporated herein in their entirety.

Colchicine. In one embodiment, a compound of the invention is administered before, concurrently or subsequent to administration of colchicine. Colchicine inhibits uric acid crystal deposition, possibly by inhibiting oxidation of glucose and subsequent lactic acid production in leukocytes. Colchicine is available in tablet form and is usually taken every two to six hours. Colchicine can be administered in prodrug form.

Opioid agents. In one embodiment, a compound of the invention is administered before, concurrently or subsequent to administration of an opioid analgesic. Opioids act as agonists, interacting, with stereo specific and saturable binding sites in the brain and other tissues. Endogenous opioid-like peptides are present particularly in areas of the central nervous system that are presumed to be related to the perception of pain; to movement, mood and behavior, and to the regulation of neuroendocrinological functions. Opioid analgesics include, for example, morphine, heroin, hydromorphone, oxymorphone, levorphanol, levallorphan, methadone, meperidine, fentanyl, cocaine, codeine, dihydrocodeine, oxycodone, hydrocodone, propoxyphene, nalmefene, nalorphine, naloxone, naltrexone, buprenorphine, butorphanol, nalbuphine and pentazocine.

Cytokines. In one embodiment, a compound of the invention is administered before, concurrently or subsequent to administration of a modulator of one or more cytokines. Cytokines can be involved in an inflammatory response. Cytokines include, without limitation, BDNF, CREB pS133, CREB Total, DR-5, EGF, ENA-78, Eotaxin, Fatty Acid Binding Protein, FGF-basic, granulocyte colony-stimulating factor (G-CSF), GCP-2, Granulocyte-macrophage Colony-stimulating Factor GM-CSF (GM-CSF), growth-related oncogene-keratinocytes (GRO-KC), HGF, ICAM-1, IFN-alpha, IFN-gamma, the interleukins IL-10, IL-11, IL-12, IL-12 p40, IL-12 p40/p70, IL-12 p70, IL-13, IL-15, IL-16, IL-17, IL-18, IL-1alpha, IL-1beta, IL-1ra, IL-1ra/IL-1F3, IL-2, IL-3, IL-4, IL-5, IL-6, IL-7, IL-8, IL-9, interferon-inducible protein (10 IP-10), JE/MCP-1, keratinocytes (KC), KC/GROa, LIF, Lymphotacin, M-CSF, monocyte chemoattractant protein-1 (MCP-1), MCP-1 (MCAF), MCP-3, MCP-5, MDC, MIG, macrophage inflammatory (MIP-1 alpha), MIP-1 beta, MIP-1 gamma, MIP-2, MIP-3 beta, OSM, PDGF-BB, regulated upon activation, normal T cell expressed and secreted (RANTES), Rb (pT821), Rb (total), Rb pSpT249/252, Tau (pS214), Tau (pS396), Tau (total), Tissue Factor, tumor necrosis factor-alpha (TNF-alpha), TNF-beta, TNF-RI, TNF-RII, VCAM-1, and VEGF. In some embodiments, the cytokine is IL-12p70, IL-10, IL-1 alpha, IL-3, IL-12 p40, IL-1ra, IL-12, IL-6, IL-4, IL-18, IL-10, IL-5, eotaxin, IL-16, MIG, IL-8, IL-17, IL-7, IL-15, IL-13, IL-2R (soluble), IL-2, LIF/HILDA, IL-1 beta, Fas/CD95/Apo-1, or MCP-1. Modulation can comprise up or downregulating the biological action of the one or more cytokines. For example, gout can be treated by inhibiting IL-1. Inhibitors can comprise small molecules, peptides, proteins or the like. Alternately, serum uric acid levels can be decreased by administration of IL-6, or fragments, conjugates or mimetics thereof. In one embodiment, a compound of the invention is administered before, concurrently or subsequent to administration of a modulator of one or more IL-1 antagonists. In one embodiment, a compound of the invention is administered before, concurrently or subsequent to administration of a modulator of IL-6, or a fragment, conjugate or mimetic thereof.

Table 2 lists therapeutic agents for treatment of hyperuricemia, gout and related disorders. In some embodiments, a compound of the invention is administered before, concurrently or subsequent to administration of one or more of the compounds listed in Table 2.

TABLE 2
Therapeutic Agents for Gout
[Data from PROUS SCIENCE INTEGRITY ®]

| | Drug Name (CD, | Mechanism | |
|---|---|---|---|
| Chemical Name/Description | GN, BN) | of Action | Related Basic Patent |
| 1-[3-Cyano-4-(2,2- | Y-700, | Xanthine Oxidase | JP 2007210978; WO |
| dimethylpropoxy)phenyl]-1H- | Piraxostat | Inhibitors | 1998018765; WO 2004080486; |
| pyrazole-4-carboxylic acid | | | WO 2007097403; WO |
| | | | 2008010315 |
| 1H-Pyrazolo[3,4- | NSC-76239, | Xanthine Oxidase | DE 1814082; U.S. Pat. No. 3,474,098; U.S. Pat. No. |
| d]pyrimidine-4,6-diol | Oxipurinol; | Inhibitors | 3,624,205; WO 2005027887; |
| | Oxypurinol, | | WO 2005030138 |
| | Oxyprim | | |
| 4-Amino-6-hydroxy-2,3- | Hydroxyakalone | Xanthine Oxidase | JP 1996188580 |
| dihydro-1H-pyrazolo[3,4- | | Inhibitors | |
| d]pyrimidin-3-one | | | |
| 5-Hydroxy-3,4,7-triphenyl- | | Xanthine Oxidase | |
| 2,6-benzofurandione | | Inhibitors | |
| 5-Amino-1-(3-cyano-4- | | Xanthine Oxidase | WO 1998018765 |
| isobutoxyphenyl)pyrazole-4- | | Inhibitors | |
| carboxylic acid ethyl ester | | | |
| 5-Amino-1-(3-cyano-4- | | Xanthine Oxidase | WO 1998018765 |
| isobutoxyphenyl)pyrazole-4- | | Inhibitors | |
| carboxylic acid | | | |
| 1-(3-Cyano-4- | | Xanthine Oxidase | WO 1998018765 |
| isobutoxyphenyl)pyrazole-4- | | Inhibitors | |
| carboxylic acid ethyl ester | | | |
| 1-(3-Cyano-4- | | Xanthine Oxidase | WO 1998018765 |
| isobutoxyphenyl)pyrazole-4- | | Inhibitors | |
| carboxylic acid | | | |
| 1-(3-Cyano-4- | | Xanthine Oxidase | WO 1998018765 |
| hexyloxyphenyl)pyrazole-4- | | Inhibitors | |
| carboxylic acid ethyl ester | | | |
| 1-(3-Cyano-4- | | Xanthine Oxidase | WO 1998018765; WO |
| hexyloxyphenyl)pyrazole-4- | | Inhibitors | 2005058307 |
| carboxylic acid | | | |
| 1-(3-Cyano-4- | | Xanthine Oxidase | WO 1998018765 |
| cyclopentyloxyphenyl)pyrazole- | | Inhibitors | |
| 4-carboxylic acid ethyl ester | | | |
| 1-(3-Cyano-4- | | Xanthine Oxidase | WO 1998018765 |
| cyclopentyloxyphenyl)pyrazole- | | Inhibitors | |
| 4-carboxylic acid | | | |
| 1-(3-Cyano-4- | | Xanthine Oxidase | WO 1998018765 |
| cyclohexyloxyphenyl)pyrazole- | | Inhibitors | |
| 4-carboxylic acid ethyl ester | | | |
| 1-(3-Cyano-4- | | Xanthine Oxidase | WO 1998018765 |
| cyclohexyloxyphenyl)pyrazole- | | Inhibitors | |
| 4-carboxylic acid | | | |
| 3-(4-Isobutoxy-3- | | Xanthine Oxidase | JP 1998310578 |
| nitrophenyl)-1H-pyrazole-5- | | Inhibitors | |
| carboxylic acid methyl ester | | | |
| 3-(3-Cyano-4- | | Xanthine Oxidase | JP 1998310578 |
| hydroxyphenyl)-1-methyl-1H- | | Inhibitors | |
| pyrazole-5-carboxylic acid | | | |
| 3-[3-Cyano-4-(3- | | Xanthine Oxidase | JP 1998310578 |
| phenylpropoxy)phenyl]-1- | | Inhibitors | |
| methyl-1H-pyrazole-5- | | | |
| carboxylic acid | | | |
| 3-[4-(Isopentyloxy)-3- | | Xanthine Oxidase | JP 1998310578 |
| nitrophenyl]-1-methyl-1H- | | Inhibitors | |
| pyrazole-5-carboxylic | | | |
| isopentyl ester | | | |
| 3-{3-Cyano-4-[2- | | Xanthine Oxidase | JP 1998310578 |
| (dimethylamino)ethoxy]phenyl}- | | Inhibitors | |
| 1-methyl-1H-pyrazole-5- | | | |
| carboxylic acid | | | |
| 4-Chloro-3-[3-cyano-4-(2,2- | | Xanthine Oxidase | JP 1998310578 |
| dimethylpropoxy)phenyl]-1- | | Inhibitors | |
| methyl-1H-pyrazole-5- | | | |
| carboxylic methyl ester | | | |
| 3-(4-Chlorophenyl)-6,7- | | Xanthine Oxidase | JP 2000038389 |
| dihydro-5H-pyrazolo[4,3- | | Inhibitors | |
| e][1,2,4]triazolo[4,3- | | | |
| c]pyrimidine-5-thione | | | |
| 3-(4-Methylphenyl)-6,7- | | Xanthine Oxidase | JP 2000038389 |
| dihydro-5H-pyrazolo[4,3- | | Inhibitors | |
| e][1,2,4]triazolo[4,3- | | | |
| c]pyrimidine-5-thione | | | |
| Benzaldehyde N-(6-thioxo- | | Xanthine Oxidase | JP 2000038389 |
| 6,7-dihydro-1H-pyrazolo[3,4- | | Inhibitors | |
| d]pyrimidin-4-yl)hydrazone | | | |
| 4-Chlorobenzaldehyde N-(6- | | Xanthine Oxidase | JP 2000038389 |
| thioxo-6,7-dihydro-1H- | | Inhibitors | |
| pyrazolo[3,4-d]pyrimidin-4- | | | |
| yl)hydrazone | | | |
| 4-Methylbenzaldehyde N-(6- | | Xanthine Oxidase | JP 2000038389 |
| thioxo-6,7-dihydro-1H- | | Inhibitors | |
| pyrazolo[3,4-d]pyrimidin-4- | | | |
| yl)hydrazone | | | |
| 4-Methoxybenzaldehyde N-(6- | | Xanthine Oxidase | JP 2000038389 |
| thioxo-6,7-dihydro-1H- | | Inhibitors | |
| pyrazolo[3,4-d]pyrimidin-4- | | | |
| yl)hydrazone | | | |
| 2-[3-Cyano-4-[3-hydroxy- | | Xanthine Oxidase | JP 2002105067 |
| 2(R)-methylpropoxy]phenyl]- | | Inhibitors | |
| 4-methylthiazole-5-carboxylic | | | |
| acid | | | |
| 2-[3-Cyano-4-(2-hydroxy-2- | | Xanthine Oxidase | JP 2002105067 |
| methylpropoxy)phenyl]-4- | | Inhibitors | |
| methylthiazole-5-carboxylic | | | |
| acid | | | |
| 2-(3-Cyano-4- | | Xanthine Oxidase | JP 2002105067 |
| hydroxyphenyl)-4- | | Inhibitors | |
| methylthiazole-5-carboxylic | | | |
| acid | | | |
| 2-[4-(2-Carboxypropoxy)-3- | | Xanthine Oxidase | JP 2002105067 |
| cyanophenyl]-4- | | Inhibitors | |
| methylthiazole-5-carboxylic | | | |
| acid | | | |
| 2-[3-Cyano-4-[3-hydroxy- | | Xanthine Oxidase | JP 2002105067 |
| 2(S)-methylpropoxy]phenyl]- | | Inhibitors | |
| 4-methylthiazole-5-carboxylic | | | |
| acid | | | |
| 8-(4-Isobutoxy-3- | | Xanthine Oxidase | WO 2003042185 |
| nitrophenyl)-9H-purin-6- | | Inhibitors | |
| amine | | | |
| 2-(4-Isobutoxy-3- | | Xanthine Oxidase | WO 2003042185 |
| nitrophenyl)oxazolo[5,4- | | Inhibitors | |
| d]pyrimidin-7-amine | | | |
| 2-(4-Isobutoxy-3- | | Xanthine Oxidase | WO 2003042185 |
| nitrophenyl)benzoxazole-7- | | Inhibitors | |
| carboxylic acid | | | |
| 2-(3-Cyano-4- | | Xanthine Oxidase | WO 2003042185 |
| isobutoxyphenyl)benzothiazole- | | Inhibitors | |
| 7-carboxylic acid | | | |
| 2-(3-Cyano-4- | | Xanthine Oxidase | WO 2003042185 |
| isobutoxyphenyl)-1H- | | Inhibitors | |
| benzimidazole-7-carboxylic | | | |
| acid | | | |
| 2-Isobutoxy-5-[3-(2- | | Xanthine Oxidase | WO 2003064410 |
| methylpyridin-4-yl)-1H-1,2,4- | | Inhibitors | |
| triazol-5-yl]benzonitrile | | | |
| 4-[3-(4-Isobutoxy-3- | | Xanthine Oxidase | WO 2003064410 |
| nitrophenyl)-1H-1,2,4-triazol- | | Inhibitors | |
| 5-yl]-2-methylpyridine | | | |
| 2-Isobutoxy-5-[5-(4-pyridyl)- | | Xanthine Oxidase | WO 2003064410 |
| 1H-1,2,4-triazol-3- | | Inhibitors | |
| yl]benzonitrile | | | |
| 4-[5-(4-Methoxy-3- | | Xanthine Oxidase | WO 2003064410 |
| nitrophenyl)-1H-1,2,4-triazol- | | Inhibitors | |
| 3-yl]-2-methylpyridine | | | |
| N-(4-tert-Butylphenyl)-4-(1H- | | Xanthine Oxidase | WO 2004009563 |
| tetrazol-5-yl)benzamide | | Inhibitors | |
| N-(4-Isobutylphenyl)-4-(1H- | | Xanthine Oxidase | WO 2004009563 |
| tetrazol-5-yl)benzamide | | Inhibitors | |
| 2-(3-Chlorophenyl)-6,7- | | Xanthine Oxidase | |
| dihydro-5H-pyrazolo[4,3- | | Inhibitors | |
| e][1,2,4]triazolo[1,5- | | | |
| c]pyrimidin-5-one | | | |
| 2-(4-Chlorophenyl)-6,7- | | Xanthine Oxidase | |
| dihydro-5H-pyrazolo[4,3- | | Inhibitors | |
| e][1,2,4]triazolo[1,5- | | | |
| c]pyrimidin-5-one | | | |
| 2-(4-Bromophenyl)-6,7- | | Xanthine Oxidase | |
| dihydro-5H-pyrazolo[4,3- | | Inhibitors | |
| e][1,2,4]triazolo[1,5- | | | |
| c]pyrimidin-5-one | | | |
| 5′-O-[2′- | | Concentrative | WO 2004101593 |
| (Trifluoromethyl)biphenyl-3- | | Nucleoside | |
| yl]adenosine | | Transporter 2 | |
| | | (CNT2) Inhibitors | |
| 5′-O-(2-Biphenylyl)adenosine | | Concentrative | WO 2004101593 |
| | | Nucleoside | |
| | | Transporter 2 | |
| | | (CNT2) Inhibitors | |
| 5′-O-(3-Biphenylyl)adenosine | | Concentrative | WO 2004101593 |
| | | Nucleoside | |
| | | Transporter 2 | |
| | | (CNT2) Inhibitors | |
| 5′-O-(3′-Chlorobiphenyl-3- | | Concentrative | WO 2004101593 |
| yl)adenosine | | Nucleoside | |
| | | Transporter 2 | |
| | | (CNT2) Inhibitors | |
| 3-(3,4-Dihydroxyphenyl)-1- | Sappanchalcone | NOS2 Expression | |
| (4-hydroxy-2- | | Inhibitors; Nitric | |
| methoxyphenyl)-2(E)-propen- | | Oxide Production | |
| 1-one | | Inhibitors; | |
| | | Xanthine Oxidase | |
| | | Inhibitors | |
| 1-Deoxy-1-[2-[2-(4- | | Concentrative | WO 2005063788 |
| hydroxybutoxy)biphenyl-4- | | Nucleoside | |
| ylmethylamino]-1H- | | Transporter 2 | |
| benzimidazol-1-yl]-beta-D- | | (CNT2) Inhibitors | |
| ribofuranose | | | |
| 1-[3-[3-[1-(beta-D- | | Concentrative | WO 2005063788 |
| Ribofuranosyl)-1H- | | Nucleoside | |
| benzimidazol-2- | | Transporter 2 | |
| ylaminomethyl]phenoxy]propyl]piperidine- | | (CNT2) Inhibitors | |
| 4-carboxamide | | | |
| 1-Deoxy-1-[2-[3-[3- | | Concentrative | WO 2005063788 |
| (dimethylamino)propoxy]benzylamino]- | | Nucleoside | |
| 1H-benzimidazol-1- | | Transporter 2 | |
| yl]-beta-D-ribofuranose | | (CNT2) Inhibitors | |
| 2-[4-(4-Chlorophenoxy)-3- | | Xanthine Oxidase | WO 2005121153 |
| nitrophenyl]thiazolol[5,4- | | Inhibitors | |
| d]pyrimidin-7-ol | | | |
| 2-(3-Nitro-4- | | Xanthine Oxidase | WO 2005121153 |
| phenoxyphenyl)thiazolo[5,4- | | Inhibitors | |
| d]pyrimidin-7-ol | | | |
| 2-[4-(4-Fluorophenoxy)-3- | | Xanthine Oxidase | WO 2005121153 |
| nitrophenyl]thiazolo[5,4- | | Inhibitors | |
| d]pyrimidin-7-ol | | | |
| 2-[4-(2-Chlorophenoxy)-3- | | Xanthine Oxidase | WO 2005121153 |
| nitrophenyl]thiazolo[5,4- | | Inhibitors | |
| d]pyrimidin-7-ol | | | |
| 2-[4-(3-Fluorophenoxy)-3- | | Xanthine Oxidase | WO 2005121153 |
| nitrophenyl]thiazolo[5,4- | | Inhibitors | |
| d]pyrimidin-7-ol | | | |
| 2-(4-Chlorophenoxy)-5-(7- | | Xanthine Oxidase | WO 2005121153 |
| hydroxythiazolo[5,4- | | Inhibitors | |
| d]pyrimidin-2-yl)benzonitrile | | | |
| 5-(3-Cyano-4- | | Xanthine Oxidase | WO 2006022375 |
| propoxyphenyl)thiophene-2- | | Inhibitors | |
| carboxylic acid sodium salt | | | |
| 5-[3-Cyano-4- | | Xanthine Oxidase | WO 2006022375 |
| (cyclopentyloxy)phenyl]-3- | | Inhibitors | |
| fluorothiophene-2-carboxylic | | | |
| acid sodium salt | | | |
| 2-[3-Cyano-4- | | 3alpha- | WO 2006022374 |
| (perhydroazepin-1- | | Hydroxysteroid | |
| yl)phenyl]pyridine-4- | | Dehydrogenase | |
| carboxylic acid hydrochloride | | type II (aldo-keto | |
| | | reductase | |
| | | AKR1C3) | |
| | | Inhibitors; | |
| | | Xanthine Oxidase | |
| | | Inhibitors | |
| 2-[3-Cyano-4-(4- | | Xanthine Oxidase | WO 2006022374 |
| methylpiperidin-1- | | Inhibitors | |
| yl)phenyl]pyridine-4- | | | |
| carboxylic acid hydrochloride | | | |
| N,N-Dimethyl-5,10-dioxo- | | | WO 2006031134 |
| 3,4,5,10-tetrahydro-2H-1,4- | | | |
| thiazino[2,3-g]quinoline-7- | | | |
| carboxamide 1,1-dioxide | | | |
| 7-Methyl-3,4-dihydro-2H-1,4- | | | WO 2006031134 |
| thiazino[2,3-g]quinoline-5,10- | | | |
| dione 1,1-dioxide | | | |
| 3,4-Dihydro-2H-naphtho[2,3- | | | WO 2006031134 |
| b]-1,4-thiazine-5,10-dione 1,1- | | | |
| dioxide | | | |
| 7-(3-Oxo-3-phenyl-1- | | | WO 2006031134 |
| propenyl)-3,4-dihydro-2H-1,4- | | | |
| thiazino[2,3-g]quinoline-5,10- | | | |
| dione 1,1-dioxide | | | |
| 5,10-Dioxo-3,4,5,10- | | | WO 2006031134 |
| tetrahydro-2H-1,4- | | | |
| thiazino[2,3-g]quinoline-7- | | | |
| carboxylic acid methyl ester | | | |
| 1,1-dioxide | | | |
| 2-[2-(4- | | Concentrative | WO 2006030803 |
| Hydroxybutoxy)biphenyl-4- | | Nucleoside | |
| ylmethylamino]adenosine | | Transporter 2 | |
| | | (CNT2) Inhibitors | |
| N-[3-[4′-(Adenosin-2- | | Concentrative | WO 2006030803 |
| ylaminomethyl)biphenyl-3- | | Nucleoside | |
| yloxy]propyl]-L-tyrosinamide | | Transporter 2 | |
| | | (CNT2) Inhibitors | |
| 2-[4′-(3- | | Concentrative | WO 2006030803 |
| Aminopropoxy)biphenyl-3- | | Nucleoside | |
| ylmethylamino]adenosine | | Transporter 2 | |
| | | (CNT2) Inhibitors | |
| 2-Chloro-4-(3,4-dihydro-2H- | | Urate Transporter | WO 2006057460 |
| 1,4-benzoxazin-4- | | 1 (URAT1) | |
| ylcarbonyl)phenol | | Inhibitors | |
| 2,6-Dichloro-4-(6-nitro-3,4- | | Urate Transporter | WO 2006057460 |
| dihydro-2H-1,4-benzoxazin-4- | | 1 (URAT1) | |
| ylcarbonyl)phenol | | Inhibitors | |
| 2-Bromo-4-(3,4-dihydro-2H- | | Urate Transporter | WO 2006057460 |
| 1,4-benzoxazin-4- | | 1 (URAT1) | |
| ylcarbonyl)phenol | | Inhibitors | |
| 2,6-Dichloro-4-(6-methoxy- | | Urate Transporter | WO 2006057460 |
| 3,4-dihydro-2H-1,4- | | 1 (URAT1) | |
| benzoxazin-4- | | Inhibitors | |
| ylcarbonyl)phenol | | | |
| 2,6-Dichloro-4-(6-fluoro-3,4- | | Urate Transporter | WO 2006057460 |
| dihydro-2H-1,4-benzoxazin-4- | | 1 (URAT1) | |
| ylcarbonyl)phenol | | Inhibitors | |
| 1-[3-[2-[1-(beta-D- | | Nucleoside | WO 2006115137 |
| Ribofuranosyl)-1H- | | Transporters | |
| benzimidazol-2- | | Inhibitors | |
| ylaminomethyl]-1-benzothien- | | | |
| 6-yloxy]propyl]piperidine-4- | | | |
| carboxamide | | | |
| 1-[2-[6-[3-[N-(2- | | Nucleoside | WO 2006115137 |
| Hydroxyethyl)-N- | | Transporters | |
| methylamino]propoxy]-1- | | Inhibitors | |
| benzothien-2-ylmethylamino]- | | | |
| 1H-benzimidazol-1-yl]-beta- | | | |
| D-ribofuranose | | | |
| 1-[2-[6-[3- | | Nucleoside | WO 2006115137 |
| (Dimethylamino)propoxy]naphthalen- | | Transporters | |
| 2-ylmethylamino]-1H- | | Inhibitors | |
| benzimidazol-1-yl]-beta-D- | | | |
| ribofuranose | | | |
| 1-[2-[6-[3-(2- | | Nucleoside | WO 2006115137 |
| Hydroxyethylamino)propoxy]- | | Transporters | |
| 1-benzofuran-2- | | Inhibitors | |
| ylmethylamino]-1H- | | | |
| benzimidazol-1-yl]-beta-D- | | | |
| ribofuranose | | | |
| 2-[3-[2-[1-(beta-D- | | Nucleoside | WO 2006115137 |
| Ribofuranosyl)-1H- | | Transporters | |
| benzimidazol-2- | | Inhibitors | |
| ylaminomethyl]-1-benzofuran- | | | |
| 6-yloxy]propylamino]-2- | | | |
| methylpropionamide | | | |
| 2-(3-Fluorophenylsulfanyl)-5- | | Xanthine Oxidase | WO 2007004688 |
| (thiazolo[5,4-d]pyrimidin-2- | | Inhibitors | |
| yl)benzonitrile | | | |
| 2-(2-Fluorophenoxy)-5- | | Xanthine Oxidase | WO 2007004688 |
| (thiazolo[5,4-d]pyrimidin-2- | | Inhibitors | |
| yl)benzonitrile | | | |
| 2-(3-Fluorophenoxy)-5- | | Xanthine Oxidase | WO 2007004688 |
| (thiazolo[5,4-d]pyrimidin-2- | | Inhibitors | |
| yl)benzonitrile | | | |
| 2-(4-Chlorophenoxy)-5- | | Xanthine Oxidase | WO 2007004688 |
| (thiazolo[5,4-d]pyrimidin-2- | | Inhibitors | |
| yl)benzonitrile | | | |
| 3,4,5-Trihydroxybenzoic acid | Ellagic acid-4- | Xanthine Oxidase | JP 2007045784 |
| 3,7,8-trihydroxy-5,10-dioxo- | gallate | Inhibitors | |
| 5,10-dihydro-1- | | | |
| benzopyran[5,4,3-cde]-1- | | | |
| benzopyran-2-yl ester | | | |
| 2-(2-Cyanobiphenyl-4- | | Xanthine Oxidase | WO 2007043457; WO |
| yl)thiazole-5-carboxylic acid | | Inhibitors | 2008126770 |
| 2-(2-Cyanobiphenyl-4- | | Xanthine Oxidase | WO 2007043457 |
| yl)pyridine-4-carboxylic acid | | Inhibitors | |
| sodium salt | | | |
| 5-(2-Cyanobiphenyl-4- | | Xanthine Oxidase | WO 2007043457 |
| yl)thiophene-2-carboxylic acid | | Inhibitors | |
| sodium salt | | | |
| 2-(2-Cyanobiphenyl-4-yl)-4- | | Xanthine Oxidase | WO 2007043457 |
| methylthiazole-5-carboxylic | | Inhibitors | |
| acid | | | |
| 2-(2-Cyano-4′- | | Xanthine Oxidase | WO 2007043457 |
| methylbiphenyl-4-yl)-4- | | Inhibitors | |
| methylthiazole-5-carboxylic | | | |
| acid sodium salt | | | |
| 2-(2-Cyano-4′- | | Xanthine Oxidase | WO 2007043457 |
| methoxybiphenyl-4-yl)-4- | | Inhibitors | |
| methylthiazole-5-carboxylic | | | |
| acid | | | |
| 1-(2-Cyanobiphenyl-4-yl)-1H- | | Xanthine Oxidase | WO 2007043457; WO |
| pyrazole-4-carboxylic acid | | Inhibitors | 2008126772 |
| 1-(2-Cyano-4′- | | Xanthine Oxidase | WO 2007043457 |
| methoxybiphenyl-4-yl)-1H- | | Inhibitors | |
| pyrazole-4-carboxylic acid | | | |
| 2-(2-Cyano-4′- | | Xanthine Oxidase | WO 2007043457 |
| methoxybiphenyl-4- | | Inhibitors | |
| yl)pyridine-4-carboxylic acid | | | |
| sodium salt | | | |
| 4-[3-Cyano-4-(2- | XO-B395-1 | Xanthine Oxidase | WO 2007043401 |
| methylphenyl)-1H-pyrrol-1- | | Inhibitors | |
| yl]benzoic acid | | | |
| 4-[3-Cyano-4-(2-thienyl)-1H- | XO-B401 | Xanthine Oxidase | WO 2007043401 |
| pyrrol-1-yl]benzoic acid | | Inhibitors | |
| 4-[3-Cyano-4-(3-thienyl)-1H- | XO-B422 | Xanthine Oxidase | WO 2007043401 |
| pyrrol-1-yl]benzoic acid | | Inhibitors | |
| 4-[3-Cyano-4-(4- | XO-B418 | Xanthine Oxidase | WO 2007043401 |
| methoxyphenyl)-1H-pyrrol-1- | | Inhibitors | |
| yl]benzoic acid | | | |
| 4-[5-Cyano-1-(4- | XO-TT271 | Xanthine Oxidase | WO 2007043400 |
| fluorophenyl)-1H-pyrazol-3- | | Inhibitors | |
| yl]benzoic acid | | | |
| 4-[5-(Benzamido)-1H- | XO-CH85 | Xanthine Oxidase | WO 2007043400 |
| pyrazol-3-yl]benzoic acid | | Inhibitors | |
| N-[3-[4-(1H-Tetrazol-5- | XO-CH83 | Xanthine Oxidase | WO 2007043400 |
| yl)phenyl]-1H-pyrazol-5- | | Inhibitors | |
| yl]benzamide | | | |
| 4-[4-[3-[4-(1H-Tetrazol-5- | XO-CH47 | Xanthine Oxidase | WO 2007043400 |
| yl)phenyl]-1H-pyrazol-5- | | Inhibitors | |
| yl]phenyl]morpholine | | | |
| 3-(8-Isopropyl-3-oxo-2- | | Urate Transporter | WO 2007086504 |
| phenyl-2,3-dihydro-4H-1,4- | | 1 (URAT1) | |
| benzoxazin-4-yl)propionic | | Inhibitors | |
| acid | | | |
| 3-[2(S)-Isopropyl-6,8- | | Urate Transporter | WO 2007086504 |
| dimethyl-3-oxo-2,3-dihydro- | | 1 (URAT1) | |
| 4H-1,4-benzoxazin-4- | | Inhibitors | |
| yl]propionic acid | | | |
| 3-[2(R)-Isopropyl-6,8- | | Urate Transporter | WO 2007086504 |
| dimethyl-3-oxo-2,3-dihydro- | | 1 (URAT1) | |
| 4H-1,4-benzoxazin-4- | | Inhibitors | |
| yl]propionic acid | | | |
| 3-[2(R)-Isobutyl-6,8-dimethyl- | | Urate Transporter | WO 2007086504 |
| 3-oxo-2,3-dihydro-4H-1,4- | | 1 (URAT1) | |
| benzoxazin-4-yl]propionic | | Inhibitors | acid |
| 3-(6,8-Dichloro-2-isopropyl-7- | | Urate Transporter | WO 2007086504 |
| methyl-3-oxo-2,3-dihydro-4H- | | 1 (URAT1) | |
| 1,4-benzoxazin-4-yl)propionic | | Inhibitors | |
| acid | | | |
| 3-(6-Ethyl-2-isopropyl-8- | | Urate Transporter | WO 2007086504 |
| methyl-3-oxo-3,4-dihydro-2H- | | 1 (URAT1) | |
| 1,4-benzoxazin-4-yl)propionic | | Inhibitors | |
| acid | | | |
| 3-(6,8-Dimethyl-3-oxo-2- | | Urate Transporter | WO 2007086504 |
| propyl-3,4-dihydro-2H-1,4- | | 1 (URAT1) | |
| benzoxazin-4-yl)propionic | | Inhibitors | |
| acid | | | |
| 3-[6-Chloro-2(R)-isopropyl-8- | | Urate Transporter | WO 2007086504 |
| methyl-3-oxo-3,4-dihydro-2H- | | 1 (URAT1) | |
| 1,4-benzoxazin-4-yl]propionic | | Inhibitors | |
| acid | | | |
| 3-[6,8-Dichloro-2(R)- | | Urate Transporter | WO 2007086504 |
| isopropyl-3-oxo-3,4-dihydro- | | 1 (URAT1) | |
| 2H-1,4-benzoxazin-4- | | Inhibitors | |
| yl]propionic acid | | | |
| 4-Amino-6-(biphenyl-4- | | Concentrative | JP 2007277203 |
| ylmethylamino)-7-(beta-D- | | Nucleoside | |
| ribofuranosyl)-7H- | | Transporter 2 | |
| pyrrolo[2,3-d]pyrimidine-5- | | (CNT2) Inhibitors | |
| carbonitrile | | | |
| 4-Amino-6-[3′-[3- | | Concentrative | JP 2007277203 |
| (dimethylamino)propoxy]biphenyl- | | Nucleoside | |
| 4-ylmethylamino]-7- | | Transporter 2 | |
| (beta-D-ribofuranosyl)-7H- | | (CNT2) Inhibitors | |
| pyrrolo[2,3-d]pyrimidine-5- | | | |
| carbonitrile | | | |
| (+)-3(R)-(3,4- | Sappanone B | Xanthine Oxidase | |
| Dihydroxybenzyl)-3,7- | | Inhibitors | |
| dihydroxy-3,4-dihydro-2H-1- | | | |
| benzopyran-4-one | | | |
| 5-[3-(4-Pyridazinyl)-1H-1,2,4- | | Xanthine Oxidase | JP 2008088107 |
| triazol-5-yl]-2- | | Inhibitors | |
| (trifluoromethoxy)benzonitrile | | | |
| 1-(3,5-Dichloro-4- | | Urate Transporter | WO 2008062740 |
| hydroxyphenyl)-1-(6-methyl- | | 1 (URAT1) | |
| 4H-1,4-benzoxazin-4- | | Inhibitors | |
| yl)methanone | | | |
| 1-(4H-1,4-Benzoxazin-4-yl)- | | Urate Transporter | WO 2008062740 |
| 1-(3,5-dichloro-4- | | 1 (URAT1) | |
| hydroxyphenyl)methanone | | Inhibitors | |
| 1-(6-Chloro-4H-1,4- | | Urate Transporter | WO 2008062740 |
| benzoxazin-4-yl)-1-(3,5- | | 1 (URAT1) | |
| dichloro-4- | | Inhibitors | |
| hydroxyphenyl)methanone | | | |
| 1-(4H-1,4-Benzoxazin-4-yl)- | | Urate Transporter | WO 2008062740 |
| 1-(3,5-dibromo-4- | | 1 (URAT1) | |
| hydroxyphenyl)methanone | | Inhibitors | |
| 1-(6-Chloro-4H-1,4- | | Urate Transporter | WO 2008062740 |
| benzoxazin-4-yl)-1-(3,5- | | 1 (URAT1) | |
| dibromo-4- | | Inhibitors | |
| hydroxyphenyl)methanone | | | |
| 1-(3,5-Dichloro-4- | | Urate Transporter | WO 2008062740 |
| hydroxyphenyl)-1-(6- | | 1 (URAT1) | |
| methoxy-4H-1,4-benzoxazin- | | Inhibitors | |
| 4-yl)methanone | | | |
| 1-(3,5-Dibromo-4- | | Urate Transporter | WO 2008062740 |
| hydroxyphenyl)-1-(6-methyl- | | 1 (URAT1) | |
| 4H-1,4-benzoxazin-4- | | Inhibitors | |
| yl)methanone | | | |
| 1-(3,5-Dibromo-4- | | Urate Transporter | WO 2008062740 |
| hydroxyphenyl)-1-[6- | | 1 (URAT1) | |
| (trifluoromethyl)-4H-1,4- | | Inhibitors | |
| benzoxazin-4-yl]methanone | | | |
| 1-(3,5-Dibromo-4- | | Urate Transporter | WO 2008062740 |
| hydroxyphenyl)-1-(6-fluoro- | | 1 (URAT1) | |
| 4H-1,4-benzoxazin-4- | | Inhibitors | |
| yl)methanone | | | |
| 2-[3-Cyano-4-(2,2- | | Xanthine Oxidase | WO 2008072658 |
| dimethylpropoxy)phenyl]pyridine- | | Inhibitors | |
| 3-carboxylic acid | | | |
| 2-[3-Cyano-4-[2-[2- | | Xanthine Oxidase | WO 2008072658 |
| (trifluoromethyl)phenyl]ethoxy]phenyl]pyridine- | | Inhibitors | |
| 3- | | | |
| carboxylic acid | | | |
| 2-[3-Cyano-4-(4- | | Xanthine Oxidase | WO 2008072658 |
| phenylbutoxy)phenyl]pyridine- | | Inhibitors | |
| 3-carboxylic acid | | | |
| 2-[3-Cyano-4-(3,3- | | Xanthine Oxidase | WO 2008072658 |
| dimethylbutoxy)phenyl]pyridine- | | Inhibitors | |
| 3-carboxylic acid | | | |
| 2-[4-[2-(2- | | Xanthine Oxidase | WO 2008072658 |
| Chlorophenyl)ethoxy]-3- | | Inhibitors | |
| cyanophenyl]pyridine-3- | | | |
| carboxylic acid | | | |
| 1-Deoxy-2-C-methyl-1-[2-[2- | | Concentrative | JP 2008214305 |
| (4-hydroxybutoxy)biphenyl-4- | | Nucleoside | |
| ylmethylamino]-1H- | | Transporter 2 | |
| benzimidazol-1-yl]-beta-D- | | (CNT2) Inhibitors | |
| ribofuranose | | | |
| 4(R)-[2-[2-(4- | | Concentrative | JP 2008214305 |
| Hydroxybutoxy)biphenyl-4- | | Nucleoside | |
| ylmethylamino]-1H- | | Transporter 2 | |
| benzimidazol-1-yl]-1(R)- | | (CNT2) Inhibitors | |
| (hydroxymethyl)bicyclo[3.1.0]hexane- | | | |
| 2(R),3(S)-diol | | | |
| 5,7-Dihydroxy-2-(4- | Acacetin | Melanin | |
| methoxyphenyl)-4H-1- | | Inhibitors; | |
| benzopyran-4-one; 5,7- | | Xanthine Oxidase | |
| Dihydroxy-4′- | | Inhibitors | |
| methoxyflavone; 4′-O- | | | |
| Methylapigenin | | | |
| 4-[3-[1(R)-Carboxy-2-(3,4- | Lithospermic | MMP-2 | |
| dihydroxyphenyl)ethoxy]-3- | acid | (Gelatinase A) | |
| oxoprop-1(E)-enyl]-2(S)-(3,4- | | Inhibitors; HIV | |
| dihydroxyphenyl)-7-hydroxy- | | Integrase | |
| 2,3-dihydro-1-benzofuran- | | Inhibitors; | |
| 3(S)-carboxylic acid | | Xanthine Oxidase | |
| | | Inhibitors; | |
| | | Antioxidants | |
| 4-[3-(4-Pyridyl)-1H-1,2,4- | FYX-051 | Xanthine Oxidase | WO 2003064410; WO |
| triazol-5-yl]pyridine-2- | | Inhibitors | 2007148835 |
| carbonitrile | | | |
| 2-[2-(2- | | Xanthine Oxidase | WO 2003064410 |
| Methoxyethoxy)ethoxy]-5-[3- | | Inhibitors | |
| (2-methylpyridin-4-yl)-1H- | | | |
| 1,2,4-triazol-5-yl]benzonitrile | | | |
| | AN-02-60 | Xanthine Oxidase | |
| | | Inhibitors | |
| (1S,3R,4R,5R)-3,4-Bis[3-(3,4- | 4,5- | Aldose Reductase | |
| dihydroxyphenyl)-2- | Dicaffeoylquinic | Inhibitors; | |
| propenoyloxy]-1,5- | acid methyl ester | Xanthine Oxidase | |
| dihydroxycyclohexanecarboxylic | | Inhibitors | |
| acid methyl ester | | | |
| | ALTU-242 | | |
| (±)-8-[3-Methoxy-4- | BOF-4272 | Xanthine Oxidase | EP 0269859; EP 0414200; JP |
| (phenylsulfinyl)phenyl]pyrazolo[1, | | Inhibitors | 2003201255; WO 2005030138 |
| 5-a]-1,3,5-triazin-4-ol | | | |
| sodium salt hydrate | | | |
| Urate oxidase (uricase) from | Uricase-PEG 20 | | WO 2003011211 |
| Candida utilis conjugated with | | | |
| polyethylene glycol of 20 kDa | | | |
| | RDEA-594 | Urate Transporter | |
| | | 1 (URAT1) | |
| | | Inhibitors | |
| Humanized IgG2 monoclonal | XOMA-052; | Anti-IL-1beta | |
| antibody against human | XMA-005.2 | | |
| interleukin-1beta | | | |
| 4-[2-[5-Bromo-4-(4- | 0481806; | Reverse | US 2006270725; WO |
| cyclopropylnaphthalen-1-yl)- | RDEA-806; AR- | Transcriptase | 2006026356; WO 2007050087 |
| 4H-1,2,4-triazol-3- | 806 | Inhibitors | |
| ylsulfanyl]acetamido]-3- | | | |
| chlorobenzoic acid potassium | | | |
| salt | | | |
| Tetramer alpha4 of des-(1-5)- | Pegloticase; | | U.S. Pat. No. 6,576,235 |
| [6-threonine,45-threonine,290- | PEG-Uricase, | | |
| lysine, 300-serine]uricase (EC | Puricase; | | |
| 1.7.3.3, urate oxidase) from | Krystexxa | | |
| Sus scrofa (porcine), non | | | |
| acetylated, of which some of | | | |
| the lysine 6-amine residues are | | | |
| engaged in a carbamate | | | |
| linkage with a monomethylic | | | |
| ether of polyoxyethylene | | | |
| (macrogol); Urate oxidase | | | |
| (synthetic Sus scrofa | | | |
| domestica variant pigKS- | | | |
| DELTAN subunit), | | | |
| homotetramer, compound with | | | |
| poly(oxy-1,2-ethanediyl); | | | |
| Des-(1-5)-[6-threonine,45- | | | |
| threonine,290-lysine,300- | | | |
| serine]uricase (EC 1.7.3.3, | | | |
| urate oxidase) from Sus scrofa | | | |
| (Pig), tetramer, compound | | | |
| with poly(oxy-1,2-ethanediyl); | | | |
| Polyethylene glycol (PEG) | | | |
| conjugated recombinant | | | |
| porcine urate oxidase | | | |
| Immunoglobulin G1, anti- | ACZ-885, | Anti-IL-1beta | WO 2002016436; WO |
| (human interleukin 1beta) | Canakinumab | | 2006119942; WO 2007050607; |
| (human clone ACZ885 heavy | | | WO 2007120828; WO |
| chain V region); | | | 2008145664 |
| Immunoglobulin G1, anti- | | | |
| (human interleukin-1 beta (IL- | | | |
| 1beta)) human monoclonal | | | |
| ACZ885; (1Glu > Glp)- | | | |
| gamma1 heavy chain (221-214′)- | | | |
| disulfide with kappa | | | |
| light chain, dimer (227-227″: | | | |
| 230-230″)-bisdisulfide; | | | |
| Immunoglobulin G1, anti- | | | |
| [Homo sapiens interleukin | | | |
| 1beta (IL-1B)] human | | | |
| monoclonal ACZ885; | | | |
| gamma1 heavy chain (Homo | | | |
| sapiens VH-IGHG1*03) (221-214′)- | | | |
| disulfide with kappa | | | |
| light chain (Homo sapiens V- | | | |
| KAPPA-IGKC*01); (227-227″: | | | |
| 230-230″)-bisdisulfide | | | |
| dimer | | | |
| N-[1,2,3,10-Tetramethoxy-9- | MPC-004, | Tubulin | WO 2002056872; WO |
| oxo-5,6,7,9- | Colchicine, | polymerization | 2003045153; WO 2006138518 |
| tetrahydrobenzo[a]heptalen- | Colstat | inhibitors; | |
| 7(S)-yl]acetamide | | Antimitotic Drugs | |
| 4-Benzamido-5- | Proglumetacin | Non-Steroidal | |
| (dipropylamino)-5- | maleate, Proxil; | Antiinflammatory | |
| oxopentanoic acid 3-[4-[2-[2- | Protaxon | Drugs | |
| [1-(4-chlorobenzoyl)-5- | | | |
| methoxy-2-methyl-1H-indol- | | | |
| 3-yl]acetoxy]ethyl]piperazin- | | | |
| 1-yl]propyl ester dimaleate | | | |
| Combination of | Betamethasone | | |
| betamethasone dipropionate | dipropionate/ | | |
| and betamethasone sodium | betamethasone | | |
| phosphate | sodium | | |
| | phosphate, | | |
| | Diprophos | | |
| 4-(N,N- | Probenecid, | MRP-1 Inhibitors | JP 2005112750 |
| Dipropylsulfamoyl)benzoic | Benuryl; | | |
| acid | Benemid | | |
| 1,2-Diphenyl-4-[2- | Sulphinpyrazone; | | FR 2026129; U.S. Pat. No. 4,288,602 |
| (phenylsulfinyl)ethyl]pyrazolidine- | Sulfinpyrazone, | | |
| 3,5-dione | Anturan; Enturen | | |
| 1,5-Dihydro-4H-pyrazolo[3,4- | BW-56-158; | Xanthine Oxidase | EP 1862166; JP 2006036700; |
| d]pyrimidin-4-one | NSC-1390, | Inhibitors | WO 2000021509; WO |
| | Allopurinol, | | 2005030138; WO 2006012438; |
| | Zyloprim; | | WO 2006096759; WO |
| | Zyloric; | | 2007018687 |
| | Aloprim; | | |
| | Lopurin | | |
| 5-(Dimethylamino)-9-methyl- | DuP-141; AZP; | Non-Steroidal | |
| 2-propyl-1H-pyrazolo[1,2- | NSC-102824; | Antiinflammatory | |
| a][1,2,4]benzotriazine- | MI-85; AHR- | Drugs; Xanthine | |
| 1,3(2H)-dione | 3018, Apazone; | Oxidase Inhibitors | |
| | Azapropazone, | | |
| | Rheumox | | |
| 2(S)-(6-Methoxy-2- | RS-3650, | Non-Steroidal | EP 0587065; EP 1064967; EP |
| naphthyl)propionic acid | Naproxen | Antiinflammatory | 1905427; JP 2002316927; US |
| sodium salt | sodium: Aleve; | Drugs | 2008153888; US 2008181934; |
| | Naproxen; | | U.S. Pat. No. 6,451,857; WO 1993006080; |
| | Synflex; | | WO 1997014669; WO |
| | Naprosyn; | | 2000004897; WO 2001095913; |
| | Naprelan; | | WO 2002098352; WO |
| | Proxen; Anaprox | | 2004110492; WO 2005058276; |
| | | | WO 2005065721; WO |
| | | | 2005097099; WO 2005123192; |
| | | | WO 2005123193; WO |
| | | | 2006003504; WO 2006081127; |
| | | | WO 2007000842; WO |
| | | | 2007004675; WO 2007022356; |
| | | | WO 2007141018; WO |
| | | | 2007150074; WO 2008000669; |
| | | | WO 2008027963; WO |
| | | | 2008030359; WO 2008074419; |
| | | | WO 2008089296; WO |
| | | | 2008094450; WO 2009002297; |
| | | | WO 2009005172; WO |
| | | | 2009015734 |
| 2-(3-Benzoylphenyl)propionic | EN-3269; NX- | Non-Steroidal | CA 2160739; EP 0679395; EP |
| acid | 304; TQ-1011; | Antiinflammatory | 0756870; EP 1905427; EP |
| | HKT-500; HKH- | Drugs | 1935405; EP 1972336; JP |
| | 508; KPT-220; | | 2002316927; JP 2005082503; |
| | RP-19583, | | JP 2006137735; JP |
| | Ketoprofen, | | 2007007189; JP 2007204437; |
| | Mohrus Tape; | | JP 2008214311; JP |
| | Men-OM; | | 2008231081; U.S. Pat. No. 5,560,924; WO |
| | Ovuvail; Orudis; | | 1993006080; WO 1995025511; |
| | Menamin; | | WO 2000004897; WO |
| | Ketotransdel; | | 2001078721; WO 2001095913; |
| | ThermoProfen; | | WO 2002007767; WO |
| | Mohrus; Keplat; | | 2004012725; WO 2004024128; |
| | Miltax; | | WO 2005023307; WO |
| | Ketoselect; | | 2005046652; WO 2005063215; |
| | Fastum; Ibifen; | | WO 2005110482; WO |
| | Profenid; | | 2005123046; WO 2005123136; |
| | Ketocid; Epatec; | | WO 2005123772; WO |
| | Sector | | 2006006799; WO 2006021709; |
| | | | WO 2006050926; WO |
| | | | 2006090833; WO 2006090839; |
| | | | WO 2006104172; WO |
| | | | 2006116626; WO 2007049892; |
| | | | WO 2007109057; WO |
| | | | 2008030359; WO 2008066115; |
| | | | WO 2008092219; WO |
| | | | 2008106220; WO 2008115572; |
| | | | WO 2008150324; WO |
| | | | 2008150995 |
| 2-(2,6- | TP-318 (gel); | Non-Steroidal | EP 0524582; EP 0595766; EP |
| Dichlorophenylamino)phenylacetic | HDT-501; DCF- | Antiinflammatory | 0600395; EP 1064967; EP |
| acid sodium salt | 100; XP-21L; | Drugs; | 1550441; EP 1930729; EP |
| | TDS-943; DIC- | Cyclooxygenase-1 | 1964552; EP 1977734; JP |
| | 075V; 318-PW; | Inhibitors; | 2004175723; JP 2005145894; |
| | GP-45840, | Cyclooxygenase-2 | JP 2005145931; JP |
| | Diclofenac | Inhibitors; | 2005145932; JP 2005314328; |
| | sodium, | Cyclooxygenase-3 | JP 2006160707; JP |
| | Dicloreum CR; | Inhibitors | 2008056567; JP 2008061862; |
| | Dealgic; | | JP 2008143856; US |
| | Voltarol; Naboal; | | 2008206155; U.S. Pat. No. 3,558,690; U.S. Pat. No. |
| | Voltaren Ofta; | | 3,778,470; U.S. Pat. No. 4,829,088; U.S. |
| | Voltarene; | | 4,960,799; U.S. Pat. No. 5,603,929; U.S. Pat. No. |
| | Voltarol Ophtha; | | 5,653,972; U.S. Pat. No. 6,365,184; U.S. Pat. No. |
| | ProSorb-D; | | 6,488,954; WO 1995031179; |
| | Rectos; Dyloject; | | WO 1996003121; WO |
| | Doroxan; | | 1996022088; WO 1996041635; |
| | Pennsaid Plus; | | WO 1997044023; WO |
| | Voltaren lotion; | | 2001047559; WO 2002022108; |
| | Voltaren Tape; | | WO 2002043723; WO |
| | Voltaren Tape L; | | 2002066030; WO 2002074281; |
| | DicloFoam; | | WO 2003024489; WO |
| | Pennsaid; | | 2003055471; WO 2003080040; |
| | Voltaren gel; | | WO 2003080044; WO |
| | Diclofelite CR; | | 2003094905; WO 2004017954; |
| | Voltaren; | | WO 2004082672; WO |
| | Dimethaid-D | | 2004112838; WO 2005027977; |
| | | | WO 2005060955; WO |
| | | | 2005074930; WO 2005087216; |
| | | | WO 2005092387; WO |
| | | | 2005097099; WO 2005099676; |
| | | | WO 2005102306; WO |
| | | | 2005112909; WO 2005120436; |
| | | | WO 2006025923; WO |
| | | | 2006048939; WO 2006056712; |
| | | | WO 2006081127; WO |
| | | | 2006083979; WO 2006090782; |
| | | | WO 2006095363; WO |
| | | | 2006096955; WO 2006097247; |
| | | | WO 2006104172; WO |
| | | | 2006126214; WO 2006131262; |
| | | | WO 2006134406; WO |
| | | | 2007004675; WO 2007016766; |
| | | | WO 2007049892; WO |
| | | | 2007066148; WO 2007083985; |
| | | | WO 2007084418; WO |
| | | | 2007112272; WO 2007113187; |
| | | | WO 2007128412; WO |
| | | | 2007144085; WO 2008006216; |
| | | | WO 2008030359; WO |
| | | | 2008049020; WO 2008049657; |
| | | | WO 2008066115; WO |
| | | | 2008106220; WO 2008110741; |
| | | | WO 2008120131; WO |
| | | | 2008150995; WO 2009002297; |
| | | | WO 2009008946; WO |
| | | | 2009028650 |
| (Z)-5-Fluoro-2-methyl-1-[4- | MK-231, | NF-kappaB | DE 2039426; EP 1905427; JP |
| (methylsulfinyl)benzylidene]- | Sulindac, | (NFKB) | 2005247807; US 2003220266; |
| 1H-indene-3-acetic acid | Sulindal; | Activation | U.S. Pat. No. 3,647,858; U.S. Pat. No. 6,051,587; WO |
| | Imbaral; | Inhibitors; | 2000004897; WO 2001012227; |
| | Arthrocine; | ABCC1 | WO 2001035956; WO |
| | Klinoril; Clinoril | Expression | 2001095913; WO 2003037373; |
| | | Enhancers; Non- | WO 2003061713; WO |
| | | Steroidal | 2005009354; WO 2005020933; |
| | | Antiinflammatory | WO 2005054181; WO |
| | | Drugs; ABCC3 | 2006102439 |
| | | Expression | |
| | | Enhancers | |
| 1-(3,5-Dibromo-4- | L-2214; MJ- | | |
| hydroxyphenyl)-1-(2-ethyl-1- | 10061, | | |
| benzofuran-3-yl)methanone | Benzbromarone; | | |
| | Benzbromaron, | | |
| | Narcaricin; | | |
| | Desuric | | |
| 4-Hydroxy-2-methyl-2H-1,2- | CP-16171, | Non-Steroidal | CA 2119531; EP 0568026; JP |
| benzothiazine-3-[N-(2- | Piroxicam, | Antiinflammatory | 2004175723; JP 2005047906; |
| pyridyl)carboxamide]-1,1- | Felden; Baxo; | Drugs | JP 2005047907; JP |
| dioxide | Feldene Fast; | | 2005047908; JP 2006045099; |
| | Feldene Gel; | | JP 2006188500; U.S. Pat. No. 3,591,584; |
| | Feldene | | U.S. Pat. No. 5,362,758; U.S. Pat. No. 5,436,241; U.S. |
| | | | Pat. No. 5,601,843; U.S. Pat. No. 5,698,225; U.S. Pat. No. |
| | | | 6,051,587; WO 1993015718; |
| | | | WO 2005011708; WO |
| | | | 2005046654; WO 2005123046; |
| | | | WO 2006021709; WO |
| | | | 2006096955; WO 2006116626; |
| | | | WO 2008148742; WO |
| | | | 2009007764 |
| 4-[5-(4-Methylphenyl)-3- | AI-525; TPI-336 | Apoptosis | EP 11064948; EP 1525883; EP |
| (trifluoromethyl)pyrazol-1- | (crystalline); SC- | Inducers; Caspase | 1726301; EP 1743654; JP |
| yl]benzenesulfonamide | 58635; YM-177, | 3 Activators; Non- | 2005053888; JP 2008201702; |
| | Celecoxib, | Steroidal | US 2003157061; US |
| | Celebrex; | Antiinflammatory | 2005026902; US 2006178347; |
| | Celecox; | Drugs; | U.S. Pat. No. 5,563,165; U.S. Pat. No. 5,760,068; U.S. |
| | Onsenal; Solexa; | Angiogenesis | Pat. No. 5,972,986; U.S. Pat. No. 6,403,630; U.S. Pat. No. |
| | Celebra | Inhibitors; | 6,440,963; U.S. Pat. No. 6,573,290; U.S. Pat. No. |
| | | Cyclooxygenase-2 | 6,777,424; WO 1995015316; |
| | | Inhibitors | WO 1996037476; WO |
| | | | 1996041625; WO 1996041626; |
| | | | WO 1996041645; WO |
| | | | 2000000200; WO 2000013685; |
| | | | WO 2000025779; WO |
| | | | 2000032189; WO 2001015687; |
| | | | WO 2001042221; WO |
| | | | 2001045705; WO 2001045706; |
| | | | WO 2001052897; WO |
| | | | 2001087343; WO 2001091750; |
| | | | WO 2001091856; WO |
| | | | 2001094369; WO 2001095877; |
| | | | WO 2001095913; WO |
| | | | 2002005799; WO 2002005815; |
| | | | WO 2002017896; WO |
| | | | 2002017923; WO 2002022124; |
| | | | WO 2002083177; WO |
| | | | 2002087584; WO 2002102376; |
| | | | WO 2003015608; WO |
| | | | 2003015797; WO 2003015799; |
| | | | WO 2003024400; WO |
| | | | 2003024958; WO 2003049720; |
| | | | WO 2003057166; WO |
| | | | 2003059271; WO 2003059294; |
| | | | WO 2003059347; WO |
| | | | 2003061704; WO 2003065988; |
| | | | WO 2003070233; WO |
| | | | 2003088959; WO 2003094924; |
| | | | WO 2003097044; WO |
| | | | 2003099794; WO 2003101438; |
| | | | WO 2004006914; WO |
| | | | 2004010955; WO 2004014431; |
| | | | WO 2004017967; WO |
| | | | 2004037245; WO 2004037270; |
| | | | WO 2004037271; WO |
| | | | 2004041243; WO 2004043934; |
| | | | WO 2004045509; WO |
| | | | 2004056349; WO 2004058302; |
| | | | WO 2004060361; WO |
| | | | 2004061433; WO 2004069029; |
| | | | WO 2004078113; WO |
| | | | 2004093811; WO 2004093813; |
| | | | WO 2004093814; WO |
| | | | 2004093816; WO 2004093868; |
| | | | WO 2004093870; WO |
| | | | 2004093895; WO 2004093896; |
| | | | WO 2004094373; WO |
| | | | 2004100895; WO 2004103283; |
| | | | WO 2004103286; WO |
| | | | 2004105699; WO 2005000238; |
| | | | WO 2005000294; WO |
| | | | 2005000297; WO 2005007106; |
| | | | WO 2005007156; WO |
| | | | 2005009340; WO 2005009342; |
| | | | WO 2005009354; WO |
| | | | 2005013902; WO 2005016243; |
| | | | WO 2005018541; WO |
| | | | 2005018563; WO 2005018564; |
| | | | WO 2005018569; WO |
| | | | 2005020895; WO 2005020910; |
| | | | WO 2005020926; WO |
| | | | 2005021004; WO 2005025510; |
| | | | WO 2005025571; WO |
| | | | 2005037193; WO 2005039565; |
| | | | WO 2005039593; WO |
| | | | 2005041864; WO 2005041879; |
| | | | WO 2005046615; WO |
| | | | 2005049014; WO 2005051378; |
| | | | WO 2005054181; WO |
| | | | 2005077394; WO 2005084654; |
| | | | WO 2005097110; WO |
| | | | 2005099691; WO 2005123192; |
| | | | WO 2005123193; WO |
| | | | 2005123772; WO 2006011048; |
| | | | WO 2006017354; WO |
| | | | 2006036777; WO 2006051340; |
| | | | WO 2006056770; WO |
| | | | 2006079923; WO 2006083508; |
| | | | WO 2006086798; WO |
| | | | 2006096778; WO 2006097459; |
| | | | WO 2006104172; WO |
| | | | 2006109170; WO 2006113531; |
| | | | WO 2007041499; WO |
| | | | 2007055890; WO 2007103828; |
| | | | WO 2008065502; WO |
| | | | 2008077823; WO 2008085674; |
| | | | WO 2008135828; WO |
| | | | 2008135829; WO 2008135852; |
| | | | WO 2008149192; WO |
| | | | 2009010837; WO 2009010842 |
| 5-Chloro-3-[4- | L-791456; MK- | Non-Steroidal | U.S. Pat. No. 6,040,319; U.S. Pat. No. 6,441,002; WO |
| (methylsulfonyl)phenyl]-2-(6- | 663; MK-0663, | Antiinflammatory | 1998003484; WO 1999020110; |
| methylpyridin-3-yl)pyridine; | Etoricoxib, | Drugs; | WO 1999045913; WO |
| 5-Chloro-6′-methyl-3-[4- | Arcoxia; Tauxib; | Cyclooxygenase-2 | 1999059635; WO 2000012093; |
| (methylsulfonyl)phenyl]-2,3′- | Nucoxia | Inhibitors | WO 2000025779; WO |
| bipyridine | | | 2001015687; WO 2001060365; |
| | | | WO 2001087343; WO |
| | | | 2001091856; WO 2001092230; |
| | | | WO 2002005815; WO |
| | | | 2002022124; WO 2002087584; |
| | | | WO 2002089798; WO |
| | | | 2003039542; WO 2003049720; |
| | | | WO 2003088959; WO |
| | | | 2003094924; WO 2004039371; |
| | | | WO 2004045509; WO |
| | | | 2005000238; WO 2005000294; |
| | | | WO 2005000297; WO |
| | | | 2005007106; WO 2005007156; |
| | | | WO 2005018569; WO |
| | | | 2005037193; WO 2005039565; |
| | | | WO 2005051378; WO |
| | | | 2005085199; WO 2006137839 |
| 2-[2-(2-Chloro-6- | COX-189, | Cyclooxygenase-2 | WO 1999011605; WO |
| fluorophenylamino)-5- | Lumiracoxib, | Inhibitors | 2001023346; WO 2002020090; |
| methylphenyl]acetic acid | Prexige | | WO 2003020261; WO |
| | | | 2003033001; WO 2003035047; |
| | | | WO 2003037341; WO |
| | | | 2003039599; WO 2003061645; |
| | | | WO 2003072097; WO |
| | | | 2003074041; WO 2003090737; |
| | | | WO 2004045509; WO |
| | | | 2004054575; WO 2004080451; |
| | | | WO 2004093856; WO |
| | | | 2004103357; WO 2005007106; |
| | | | WO 2005007156; WO |
| | | | 2005037193; WO 2005037266; |
| | | | WO 2005039565; WO |
| | | | 2005097096; WO 2006017354; |
| | | | WO 2006100213; WO |
| | | | 2008156645; WO 2009010529 |
| Interleukin 1 receptor | IL-1 Trap, IL-1 | IL-1 Inhibitors | EP 1229047; WO 2004039951; |
| accessory protein (human | Cytokine Trap; | | WO 2004098596; WO |
| extracellular domain | Rilonacept; | | 2004098605; WO 2004100987; |
| fragment) fusion protein with | Interleukin-1 | | WO 2005117945; WO |
| type I interleukin 1 receptor | Trap, Arcalyst | | 2006023665; WO 2006076673; |
| (human extracellular domain | | | WO 2006084145; WO |
| fragment) fusion protein with | | | 2007042524; WO 2008051496 |
| immunoglobulin G1 (human | | | |
| Fc fragment), homodimer; | | | |
| [653-Glycine][human | | | |
| interleukin-1 receptor | | | |
| accessory protein-(1-339)- | | | |
| peptide (extracellular domain | | | |
| fragment) fusion protein with | | | |
| human type 1 interleukin-1 | | | |
| receptor-(5-316)-peptide | | | |
| (extracellular domain | | | |
| fragment) fusion protein with | | | |
| human immunoglobulin G1- | | | |
| (229 C-terminal residues)- | | | |
| peptide (Fc fragment)], (659-659′: | | | |
| 662-662′)-bisdisulfide | | | |
| dimer; Dimeric fusion protein | | | |
| consisting of the ligand- | | | |
| binding domains of the | | | |
| extracellular portions of the | | | |
| human interleukin-1 receptor | | | |
| component (IL-1RI) and IL-1 | | | |
| receptor accessory protein (IL- | | | |
| 1RAcP) linked in-line to the | | | |
| Fc portion of human IgG1, | | | |
| expressed in recombinant | | | |
| Chinese hamster ovary (CHO) | | | |
| cells and with a molecular | | | |
| weight of approximately 251 kDa | | | |

One of skill in the art will appreciate that a combination therapy comprising a pharmaceutical agent of the present invention, e.g., Tranilast, can further comprise a plurality of other pharmaceutically active agents as described herein. In a non-limiting example, a patient with chronic gout may be administered a xanthane oxidase inhibitor, e.g., allopurinol, and a uricosuric agent, e.g., probenecid. The patient can be treated with a pharmaceutical agent of the present invention, e.g., Tranilast, as well. Any such combinations are within the scope of the present invention.

### (b) Cardiovascular and Related Disorders

As described above, hyperuricemia is associated with any number of cardiovascular disorders, including without limitation cardiovascular disease and other conditions, including hypertension, metabolic syndrome, hyperlipidemia, insulin resistance, coronary artery disease, peripheral artery disease cerebrovascular disease, vascular dementia, preeclampsia, heart disease, congestive heart failure, atherosclerosis and kidney disease. Furthermore, high levels of uric acid can predict the onset of hypertension, obesity, diabetes and kidney disease.

In some embodiments, a compound of the present invention, e.g., Tranilast, or a pharmaceutically acceptable salt thereof, may be administered in combination with any other therapeutic agent and/or intervention that is commonly used for the treatment of cardiovascular or related disorders. Such agents include but are not limited to agents used to treat diabetes, including but not limited to agents that improve insulin sensitivity such as PPAR gamma ligands (thiazolidinedones, glitazones, troglitazones, rosiglitazone (Avandia), pioglitazone, stimulators of insulin secretion such as sulphonylureas (gliquidone, tolbutamide, glimepride, chlorpropamide, glipizide, glyburide, acetohexamide) and meglitinides (meglitinide, repaglinide, nateglinide) and agents that reduce liver production of glucose such as metformin. Such agents include but are not limited to agents used to treat vascular disease, including but not limited to endothelin receptor antagonists commonly used for the treatment of hypertension and other endothelial dysfunction-related disorders, such as bosentan, darusentan, enrasentan, tezosentan, atrasentan, ambrisentan sitaxsentan; smooth muscle relaxants such as PDE5 inhibitors (indirect-acting) and minoxidil (direct-acting); angiotensin converting enzyme (ACE) inhibitors such as captopril, enalapril, lisinopril, fosinopril, perindopril, quinapril, trandolapril, benazepril, ramipril; angiotensin II receptor blockers such as irbesartan, losartan, valsartan, eprosartan, olmesartan, candesartan, telmisartan; beta blockers such as atenolol, metoprolol, nadolol, bisoprolol, pindolol, acebutolol, betaxolol, propranolol; diuretics such as thiazide, hydrochlorothiazide, furosemide, torsemide, metolazone; calcium channel blockers such as amlodipine, felodipine, nisoldipine, nifedipine, verapamil, diltiazem; alpha receptor blockers doxazosin, terazosin, alfuzosin, tamsulosin; and central alpha agonists such as clonidine. Such agents include but are not limited to agents used to treat hyperlipidemia, including but not limited to agents that lower LDL such as statins (atovastatin, fluvastatin, lovastatin, pravastatin, rosuvastatin calcium, simvastatin) and nicotinic acid, agents that stimulate PPAR alpha such as fibrates, gemfibrozil, fenofibrate, bezafibrate, ciprofibrate, agents that bind and prevent readsorption of bile acids and reduce cholesterol levels such as bile acid sequestrants, cholestyramine and colestipol, and cholesterol absorption inhibitors. Such agents include those that reduce risk of heart attack, including COX-1 inhibitors including aspirin and NSAIDs, as described herein, or COX-2 inhibitors, also described herein.

In some embodiments, a compound of the present invention, e.g., Tranilast, or a pharmaceutically acceptable salt thereof, may be administered in combination with any other therapeutic agent and/or intervention that is commonly used for the treatment of diabetes or related disorders. In such embodiments, combination therapy can be used for modulating (preventing the onset of the symptoms or complications associated with) diabetes (or treating, preventing or reducing the risk of developing, diabetes and its related symptoms, complications, and disorders), wherein the compounds of the present invention, e.g., Tranilast, can be effectively used in combination with, for example, biguanides (such as metformin); thiazolidinediones (such as ciglitazone, pioglitazone, troglitazone, and rosiglitazone); dipeptidyl-peptidase-4 (“DPP-IV”) inhibitors (such as vildagliptin and sitagliptin); glucagonlike peptide-1 (“GLP-1”) receptor agonists (such as exanatide) (or GLP-1 mimetics); PPAR gamma agonists or partial agonists; dual PPAR alpha, PPAR gamma agonists or partial agonists; dual PPAR delta, PPAR gamma agonists or partial agonists; pan PPAR agonists or partial agonists; dehydroepiandrosterone (also referred to as DHEA or its conjugated sulphate ester, DHEA-SO.sub.4); antiglucocorticoids; TNF-alpha inhibitors; alpha-glucosidase inhibitors (such as acarbose, miglitol, and voglibose); sulfonylureas (such as chlorpropamide, tolbutamide, acetohexamide, tolazamide, glyburide, gliclazide, glynase, glimepiride, and glipizide); pramlintide (a synthetic analog of the human hormone amylin); other insulin secretogogues (such as repaglinide, gliquidone, and nateglinide); insulin (or insulin mimetics); glucagon receptor antagonists; gastric inhibitory peptide (“GIP”); or GIP mimetics.

In some embodiments, a compound of the present invention, e.g., Tranilast, or a pharmaceutically acceptable salt thereof, may be administered in combination with any other therapeutic agent and/or intervention that is commonly used for the treatment of obesity or obesity-related disorders. In some embodiments, the compounds of the present invention can be used in combination with, for example, phenylpropanolamine, phenteramine; diethylpropion; mazindol; fenfluramine; dexfenfluramine; phentiramine, beta-3 adrenoceptor agonist agents; sibutramine; gastrointestinal lipase inhibitors (such as orlistat); and leptins. Other agents used in treating obesity or obesity-related disorders wherein the compounds of the present invention can be effectively used in combination with, for example, cannabinoid-1 (“CB-1”) receptor antagonists (such as rimonabant); PPAR delta agonists or partial agonists; dual PPAR alpha, PPAR delta agonists or partial agonists; dual PPAR delta, PPAR gamma agonists or partial agonists; pan PPAR agonists or partial agonists; neuropeptide Y; enterostatin; cholecytokinin; bombesin; amylin; histamine H3 receptors; dopamine D2 receptors; melanocyte stimulating hormone; corticotrophin releasing factor; galanin; and gamma amino butyric acid (GABA).

In some embodiments, a compound of the present invention, e.g., Tranilast, or a pharmaceutically acceptable salt thereof, may be administered in combination with any other therapeutic agent and/or intervention that is commonly used for the treatment of hyperlipidemia and related complications, wherein the compounds of the present invention are used in combination with, for example, statins (such as atorvastatin, fluvastatin, lovastatin, pravastatin, and simvastatin), CETP inhibitors (such as torcetrapib); a cholesterol absorption inhibitor (such as ezetimibe); PPAR alpha agonists or partial agonists; PPAR delta agonists or partial agonists; dual PPAR alpha, PPAR delta agonists or partial agonists; dual PPAR alpha, PPAR gamma agonists or partial agonists; dual PPAR delta, PPAR gamma agonists or partial agonists; pan PPAR agonists or partial agonists; fenofibric acid derivatives (such as gemfibrozil, clofibrate, fenofibrate, and bezafibrate); bile acid-binding resins (such as colestipol or cholestyramine); nicotinic acid; probucol; betacarotene; vitamin E; or vitamin C.

In some embodiments, a compound of the present invention, e.g., Tranilast, or a pharmaceutically acceptable salt thereof, may be administered in combination with any other therapeutic agent and/or intervention that is commonly used for the treatment of atherosclerosis, wherein a compound of the present invention, e.g., Tranilast, is administered in combination with one or more of the following active agents: an antihyperlipidemic agent; a plasma HDL-raising agent; an antihypercholesterolemic agent, such as a cholesterol biosynthesis inhibitor, e.g., an hydroxymethylglutaryl (HMG) CoA reductase inhibitor (also referred to as statins, such as lovastatin, simvastatin, pravastatin, fluvastatin, and atorvastatin); an HMG-CoA synthase inhibitor; a squalene epoxidase inhibitor; or a squalene synthetase inhibitor (also known as squalene synthase inhibitor); an acyl-coenzyme A cholesterol acyltransferase (ACAT) inhibitor, such as melinamide; probucol; nicotinic acid and the salts thereof and niacinamide; a cholesterol absorption inhibitor, such as beta-sitosterol; a bile acid sequestrant anion exchange resin, such as cholestyramine, colestipol or dialkylaminoalkyl derivatives of a cross-linked dextran; an LDL receptor inducer; fibrates, such as clofibrate, bezafibrate, fenofibrate, and gemfibrizol; vitamin B6 (also known as pyridoxine) and the pharmaceutically acceptable salts thereof, such as the HCl salt; vitamin B12 (also known as cyanocobalamin); vitamin B3 (also known as nicotinic acid and niacinamide); anti-oxidant vitamins, such as vitamin C and E and beta carotene; a beta-blocker; an angiotensin II antagonist; an angiotensin converting enzyme inhibitor; PPAR alpha agonists or partial agonists; PPAR delta agonists or partial agonists; PPAR gamma agonists or partial agonists; dual PPAR alpha, PPAR delta agonists or partial agonists; dual PPAR alpha, PPAR gamma agonists or partial agonists; dual PPAR delta, PPAR gamma agonists or partial agonists; pan PPAR agonists or partial agonists; and a platelet aggregation inhibitor, such as fibrinogen receptor antagonists (i.e., glycoprotein IIb/IIIa fibrinogen receptor antagonists) and aspirin. As noted herein, the compounds of the present invention can be administered in combination with more than one additional active agent, for example, a combination of Tranilast with an HMG-CoA reductase inhibitor (e.g., atorvastatin, fluvastatin, lovastatin, pravastatin, and simvastatin) and aspirin, or a combination of Tranilast with an HMG-CoA reductase inhibitor and a blocker.

Additionally, an effective amount of a compound of the present invention and a therapeutically effective amount of one or more active agents selected from the group consisting of: an antihyperlipidemic agent; a plasma HDL-raising agent; an antihypercholesterolemic agent, such as a cholesterol biosynthesis inhibitor, for example, an HMG-CoA reductase inhibitor; an HMG-CoA synthase inhibitor; a squalene epoxidase inhibitor, or a squalene synthetase inhibitor (also known as squalene synthase inhibitor); an acyl-coenzyme A cholesterol acyltransferase inhibitor; probucol; nicotinic acid and the salts thereof; CETP inhibitors such as torcetrapib; a cholesterol absorption inhibitor such as ezetimibe; PPAR alpha agonists or partial agonists; PPAR delta agonists or partial agonists; dual PPAR alpha, PPAR delta agonists or partial agonists; dual PPAR alpha, PPAR gamma agonists or partial agonists; dual PPAR delta, PPAR gamma agonists or partial agonists; pan PPAR agonists or partial agonists; niacinamide; a cholesterol absorption inhibitor; a bile acid sequestrant anion exchange resin; a LDL receptor inducer; clofibrate, fenofibrate, and gemfibrozil; vitamin B6 and the pharmaceutically acceptable salts thereof; vitamin B12; an anti-oxidant vitamin; a beta-blocker; an angiotensin II antagonist; an angiotensin converting enzyme inhibitor; a platelet aggregation inhibitor; a fibrinogen receptor antagonist; aspirin; phentiramines, beta-3 adrenergic receptor agonists; sulfonylureas, biguanides, alpha-glucosidase inhibitors, other insulin secretogogues, and insulin can be used together for the preparation of a pharmaceutical composition useful for treatments as described herein.

In some embodiments, a compound of the present invention, e.g., Tranilast, or a pharmaceutically acceptable salt thereof; are administered in combination with any other therapeutic agent and/or intervention used for the treatment of metabolic syndrome (or treating metabolic syndrome and its related symptoms, complications and disorders), wherein the compounds of the present invention can be effectively used in combination with, for example, the active agents discussed above for modulating or treating diabetes, obesity, hyperlipidemia, atherosclerosis, and/or their respective related symptoms, complications and disorders.

In a further embodiment, a compound of the present invention can be administered in combination with halofenic acid, an ester of halofenic acid, or another prodrug of halofenic acid, preferably with (−)-(4-chlorophenyl)-(3-trifluoromethylphenoxy)-acetic acid 2-acetylaminoethyl ester (metaglidasen).

### (c) Renal Disorders

In some embodiments, a compound of the present invention, e.g., Tranilast, or a pharmaceutically acceptable salt thereof, may be administered in combination with any other therapeutic agent and/or intervention that is used for the treatment of renal or urological or related disorders, e.g., NO donors, calcium channel blockers, cholinergic modulators, alpha-adrenergic receptor antagonists, beta-adrenergic receptor agonists, phosphodiesterase inhibitors, cAMP-dependent protein kinase activators (e.g., cAMP mimetics), superoxide scavengers, potassium channel activators, estrogen-like compounds, testosterone-like compounds, benzodiazepines, adrenergic nerve inhibitors, antidiarrheal agents, HMG-CoA reductase inhibitors, smooth muscle relaxants, adenosine receptor modulators, adenylyl cyclase activators, endothelin receptor antagonists, bisphosphonates, cGMP-dependent protein kinase activators (e.g., cGMP mimetics). In some embodiments, the treatments for renal disorders comprise treatments for kidney stones. For example, the compound of the present invention can be given with muscle relaxants that assist in stone passage, including alpha adrenergic blocking agents such as Flomax, Uroxatral, terazosin or doxazosin. Pain of stones can be treated with nonsteroidal anti-inflammatories (NSAIDs) or opioids such as codeine or hydrocodone. NSAIDs and additional opioids are described herein. In some cases, thiazides, potassium citrate, magnesium citrate and allopurinol, are prescribed depending on the type of stone. For high urinary calcium, thiazides may be prescribed. For uric acid stones, allopurinol or other treatments for hyperuricemia can be used. Calgranulin may help prevent calcium oxalate kidney stone formation.

### (d) Dosing and Dosage Forms

The pharmaceutical compositions of the present invention, an the compounds of formula (II) or pharmaceutically acceptable salts thereof, may be administered by the usual routes and the dosage level depends upon the age, weight, conditions of the patient and the administration route. The compositions and compounds of the invention can be administered in a variety of dosage forms, e.g. orally, in the form of tablets, capsules, sugar or film coated tablets, liquid solutions or suspensions; rectally in the form of suppositories; parenterally, e.g. intramuscularly, or by intravenous and/or intrathecal and/or intraspinal injection or infusion.

The agents described herein, where combinational therapy is employed, do not have to be administered in the same pharmaceutical composition, and, because of different physical and chemical characteristics, are optionally administered by different routes. The initial administration is generally made according to established protocols, and then, based upon the observed effects, the dosage, modes of administration and times of administration subsequently modified.

In some embodiments, the active ingredients described above can be administered simultaneously or sequentially with a uric acid-binding polymer. In one embodiment, where the ingredients are administered simultaneously, they can optionally be bound to the polymer, for example, by covalent bonding or a hydrolyzable bonding, or by physically encapsulating the ingredient, on the exterior or interior of the polymeric particle. Covalent bonding can be accomplished by reacting the polymer and ingredient(s) with suitable cross-linking agents.

Therapeutically effective dosages vary when the drugs are used in treatment combinations. Methods for experimentally determining therapeutically-effective dosages of drugs and other agents for use in combination treatment regimens are documented methodologies. One example of such a method is the use of metronomic dosing, i.e., providing more frequent, lower doses in order to minimize toxic side effects. Combination treatment further includes periodic treatments that start and stop at various times to assist with the clinical management of the patient.

In any case, multiple therapeutic agents can be administered in any order, or even simultaneously. If simultaneously, the multiple therapeutic agents are optionally provided in a single, unified form, or in multiple forms (by way of example only, either as a single pill or as two separate pills). In some embodiments, one of the therapeutic agents is given in multiple doses, or both are given as multiple doses. If not simultaneous, the timing between the multiple doses optionally varies from more than zero weeks to less than four weeks. In addition, the combination methods, compositions and formulations are not to be limited to the use of only two agents; the use of multiple therapeutic combinations are also envisioned (e.g., 2, 3, 4 or more combinations).

It is understood that the dosage regimen to treat the condition(s) for which relief is sought, is optionally modified in accordance with a variety of factors. These factors include the condition from which the subject suffers, as well as the age, weight, sex, diet, and medical condition of the subject. Thus, the dosage regimen actually employed varies widely, in some embodiments, and therefore deviates from the dosage regimens set forth herein.

The pharmaceutical agents which make up the combination therapy disclosed herein are optionally a combined dosage form (e.g., combined in the same formulation) or in separate dosage forms (e.g., two or more different formulations) intended for substantially simultaneous administration. Simultaneous administration can be by the same route or by different routes. The pharmaceutical agents that make up the combination therapy can optionally be administered sequentially, with either therapeutic agent being administered by a regimen calling for multi-step administration. The multi-step administration regimen optionally calls for sequential administration of the active agents or spaced-apart administration of the separate active agents. By “sequential” administration is meant a time difference of from seconds, minutes, hours or days between the two or more administration steps of the two or more active ingredients. The two or more agents may be administered in any order. The time period between the multiple administration steps may depend upon the properties of each pharmaceutical agent, such as potency, solubility, bioavailability, plasma half-life and kinetic profile of the pharmaceutical agent. Circadian variation of the target molecule concentrations are optionally used to determine the optimal dose interval.

In addition, a modulatory agent (e.g., Tranilast) is optionally used in combination with procedures that provide additional or synergistic benefit to the patient. By way of example only, patients are expected to find therapeutic and/or prophylactic benefit in the methods described herein, wherein pharmaceutical compositions of a modulatory agent with other therapeutics are combined with genetic testing to determine whether that individual is a carrier of a mutant gene that is correlated with a certain disease or condition.

Compositions comprising two or more active ingredients (e.g., Tranilast and at least one other active ingredient) can be administered before, during or after the occurrence of a disease or condition, and the timing of administering the composition varies in some embodiments. Thus, for example, a composition comprising Tranilast and at least one other active ingredient can be used as a prophylactic and can be administered continuously to subjects at risk of developing a condition or disease (e.g., inflammatory bowel disease, myocardial infarction or an autoimmune disorder) in order to prevent the occurrence of the disease or condition. Said subjects may be asymptomatic. Subjects with hyperuricemia as a sole diagnosis (or together with other indicators of disease) may be treated prophylatically for diseases and conditions described herein, e.g., those related to or arising from hyperuricemia. Compositions comprising two or more active ingredients can be administered to a subject during or as soon as possible after the onset of the symptoms. For example compositions comprising two or more active ingredients can be administered within the first 48 hours of the onset of the symptoms. In some embodiments the compositions can be administered within the first 6 hours of the onset of the symptoms or within 3 hours after the onset of the symptoms. The initial administration can be via any suitable route. Compositions comprising two or more active ingredients as disclosed herein can be administered as soon as is practicable after the onset of a disease or condition is detected or suspected, and for any length of time necessary for the treatment of the disease.

Tablets, troches, pills, capsules and the like may also contain the components as listed hereafter: a binder such as gum, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavoring agent such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain one or more active ingredients, sucrose as a sweetening agent, methyl and propylparabens as preservatives, a dye and flavoring such as cherry or orange flavor. In some embodiments, additional ingredients, for example, nonsteroidal anti-inflammatory drugs or colchicine, ingredients for treating other related indications, or inert substances such as artificial coloring agents are added. Of course, any material used in preparing any dosage unit form should be pharmaceutically pure and substantially non-toxic in the amounts employed. In addition, the one or more active ingredients may be incorporated into sustained-release preparations and formulations as described herein.

Examples of therapeutic compositions or modalities that can be combined with one or more compositions or methods of the invention are disclosed relevant portions of the following U.S. Patents or Patent Application Publications: Publication Nos. 2008/0221060; 2008/0249091; 2008/0233113; 2008/0200494; 2008/0114058; 2008/0076776; 2008/0038242; 2008/0188426; U.S. Pat. Nos. 7,452,867; 7,361,671; 7,232,812; 7,186,695; 7,030,119; 6,500,459; and 6,815,464.

Additional active agents that can be selected for combination therapy according to the present invention include structurally or functionally related therapeutic agents to those disclosed herein, e.g., without limitation, prodrugs, analogs, homologs, derivatives, isomers, mimetics, metabolic derivatives, secondary metabolites, esters, or salt forms. Analogs include compounds with substituted atoms or functional groups, transition state analogs or similar structure. Isomers include, without limitation, stereoisomers, enantiomers, geometrical isomers, cis-trans isomers, conformers, rotamers, tautomers, topoisomers or constitutional (structural) isomers. A structurally related compound could be a drug modified to improve pharmacological properties or processability. For a biological therapeutic agent, this could comprise a related peptide or immunotoxin. For example, a monoclonal antibody might be used in a combination therapy of the present invention. One of skill in the art will appreciate that combination therapy according to the present invention further comprises the monoclonal antibody conjugated to one or more toxic agents. Such antibody drug conjugates are well known in the art. See, e.g., U.S. Patent Publication No. 2008/0025989, filed Apr. 13, 2007 and entitled “Anti-Cd70 Antibody-Drug Conjugates and Their Use for the Treatment of Cancer and Immune Disorders.” Further, the present invention envisions the use of a peptide mimetic.

While the present invention has been described in conjunction with the specific embodiments set forth above, many alternatives, modifications and variations thereof will be apparent to those of ordinary skill in the art. Thus, for example, classes of known uricosuric or anti-inflammatory agents not recited above are within the scope of the invention, as are known but unrecited species of recited classes of uricosuric or anti-inflammatory agents. Similarly, the treatment of known but unrecited gouty conditions is within the scope of the invention. All such alternatives, modifications, and variations are intended to fall within the spirit and scope of the present invention.

The pharmaceutical compositions of the present invention, as well as the compounds of formula II, or pharmaceutically acceptable salts thereof, may be administered once daily (QD), twice daily (BID), three times daily (TID) or four times per day (QID). In one embodiment, the pharmaceutical composition of the present invention, or the compound of formula II, or pharmaceutically acceptable salt thereof, is administered once daily (QD). In another embodiment, the pharmaceutical composition of the present invention, or the compound of formula II, or pharmaceutically acceptable salt thereof, is administered twice daily (BID).

### IV. Methods of Use

### (a) Treatments of Hyperuricemic Disorders

A common target of treatment of gout aims to relieve pain and inflammation of the acute attack, and reduce the incidence of recurrent attacks. Dietary and pharmacological urate-lowering therapies principally aim to prevent or reverse uric acid crystal formation and clinical joint damage. Common approaches to the treatment of acute gout include corticosteroids, non-steroidal anti-inflammatory drugs (NSAIDs), and colchicine. The side effects of these drugs, particularly in the frail, elderly population who experience the highest incidence of acute gout, can be serious. An approach to the prevention of recurrent gout is the use of a xanthine oxidase inhibitor, allopurinol. However, allopurinol can have serious side effects such as allopurinol hypersensitivity syndrome. See, e.g., Arellano et al. (1993) Ann Pharmacother 27:337-343.

Lowering the serum uric acid levels results in a reduction in the frequency of gout attacks. Maintaining serum uric acid levels below 6.0 mg/dL is commonly the target of treatment for chronic gout.

The compounds of the invention, when used as a medicament, are useful in reducing uric acid in a subject, such as a mammal. The term “mammal” as used herein can include humans, primates, livestock animals (e.g., sheep, pigs, cattle, horses, donkeys), laboratory test animals (e.g., mice, rabbits, rats, guinea pigs), companion animals (e.g., dogs, cats) and captive wild animals (e.g., foxes, kangaroos, deer). The mammal can be a human or a laboratory test animal. In some embodiments, the mammal is a human. Even in embodiments exemplified with respect to laboratory test animals, this should not be understood in any way as limiting the application of the present invention to humans.

The term “subject” as used herein can be a mammal. In some embodiments, the term “subject” refers to a human. In some embodiments, the human is a patient. In some embodiments, the subject is known to suffer from a hyperuricemic disorder. For example, in some embodiments, the subject has uric acid crystal formation determined by aspiration of tophi or by aspiration of synovial fluid of an inflamed joint. In some embodiments, gouty conditions are determined by clinical criteria, e.g., podagra, tophi, or other joint pain and swellings, or an elevated serum uric acid level. Radiography techniques may also help determine whether a patient suffers from hyperuricemia, e.g., by showing evidence of joint damage or uric acid crystal formation. Such techniques include x-ray film, computed tomography (CT) scans, magnetic resonance imaging (MRI), DECT and ultrasound. In some embodiments, the subject is known to suffer from gout, e.g., by one or more prior occurrences of gouty attack. In some embodiments, the subject has severe gout. In some embodiments, the subject has refractory gout wherein prior art treatments have proven insufficient to control disease. In some embodiments, the subject has chronic gout. In some embodiments, the methods of the present invention are used to treat a patient with acute gout, e.g., presenting with a first attack comprising podagra.

In particular, the compositions of the invention are useful in controlling the level of uric acid and uric acid crystal formation in a subject suffering from gout and ameliorating symptoms related to a high level of uric acid and uric acid crystal formation such as muscle spasm, localized swelling, inflammation, joint pains, muscle fatigue, stress feelings, and myocardial infraction. Crystal formation can be in one or more of the joints, under the skin, or in the kidneys. Some deposits may be so severe as to cause tophi.

Diseases associated with high levels of serum uric acid levels include, but are not limited to, gout, hyperuricemia, urinary lithiasis, hyperuricemic nephropathy, acute uric acid nephropathy and the like, especially gout and hyperuricemia.

An “effective amount” means an amount necessary at least partly to attain the desired response, or to delay the onset or inhibit progression or halt altogether, the onset or progression of a particular condition being treated. The amount varies depending upon the health and physical condition of the subject to be treated, the taxonomic group of individual to be treated, the degree of protection desired, the formulation of the composition, the assessment of the medical situation, and other relevant factors. It is expected that the amount will fall in a relatively broad range that can be determined through routine trials.

One of skill in the art will appreciate that the effective amount may be adjusted when therapeutic agents are used in combination. For example, a compound provided by the present invention, e.g., Tranilast, or a pharmaceutically acceptable salt thereof, may be used in combination with a xanthane oxidase inhibitor, e.g., allopurinol or febuxostat. When such combinations are used, the dose of one or more of the agents may be reduced to a level below the level required for a desired efficacy when the one or more agents are used alone.

Reference herein to “treatment” and “prophylaxis” is to be considered in its broadest context. The term “treatment” does not necessarily imply that a subject is treated until total recovery. Similarly, “prophylaxis” does not necessarily mean that the subject will not eventually contract a disease condition. Accordingly, treatment and prophylaxis can include amelioration of the symptoms of a particular condition or preventing or otherwise reducing the risk of developing a particular condition. The term “prophylaxis” may be considered as reducing the severity or onset of a particular condition. “Treatment” may also reduce the severity of an existing condition. As described above, “treatment” as used herein includes prophylaxis.

Methods to determine serum uric acid levels are well known in the art and are often measured as part of a standard chemistry panel of blood serum samples. In various embodiments, the methods of the present invention lower serum uric acid levels in a subject by at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90% or more, as compared to serum uric acid levels in the subject prior to administering the methods of the present invention. In various embodiments, serum uric acid levels are decreased by at least between 5% to 50%, decreased by at least 25% to 75%, or decreased by at least 50% to 99%.

In some embodiments, the methods of the present invention lower serum uric acid levels by 0.1, 0.2, 0.3, 0.4, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5 or 10.0 mg/dL, or greater, as compared to serum uric acid levels in the subject prior to administering the methods of the present invention. In further embodiments, the methods of the present invention lower serum uric acid levels by between 0.1-10.0 mg/dL, 0.5-6.0 mg/dL, 1.0-4.0 mg/dL or 1.5-2.5 mg/dL.

In one embodiment, the methods of the present invention are used to treat a patient diagnosed with hyperuricemia, ameliorate symptoms associated with hyperuricemia, or prevent the onset of hyperuricemia by lowering or maintaining serum uric acid levels in a subject below 7.0 or 6.5 or 6.0 mg/dL, or lower. In some embodiments, the methods are used to treat or prevent gout in a subject in need thereof. In some embodiments, the methods are used to reduce the severity or number of gouty attacks in a subject in need thereof. In some embodiments, the methods are used to reduce uric acid crystal formation in a subject in need thereof. For example, the methods may ameliorate gout by reducing serum uric acid levels to an acceptable level wherein gouty attacks are less frequent or do not occur. Similarly, the methods may ameliorate gouty symptoms by reducing serum uric acid levels to a level wherein adverse affects are no longer observed.

In some embodiments, one or more other therapeutic agents are administered in combination with one or more pharmaceutical compositions of the invention or pharmaceutically acceptable salts thereof to treat hyperuricemia or the effects thereof, e.g., gout. In some embodiments, the one or more other therapeutic agents comprise a xanthine oxidase inhibitor. In some embodiments, the xanthine oxidase inhibitor is allopurinol, febuxostat, oxypurinol, tisopurine, or an inositol. In some embodiments, the one or more other therapeutic agents comprise a uricosuric agent. In some embodiments, the uricosuric agent is probenecid, benzbromarone, sulfinpyrazone, guaifenesin, losartan, atorvastatin, amlodipine, adrenocorticotropic hormone (ACTH or corticotropin), or fenofibrate. In some embodiments, the one or more other therapeutic agents comprise a uricase enzyme, or a fragment or pegylated derivative thereof. In some embodiments, the uricase enzyme is rasburicase or pegloticase. In some embodiments, the one or more other therapeutic agents comprise cortisone. In some embodiments, the one or more other therapeutic agents comprise an anti-inflammatory agent. In some embodiments, the anti-inflammatory agent is a nonsteroidal anti-inflammatory drug (NSAID). In some embodiments, the NSAID is diclofenac, indomethacin, naproxen, sulindac, lumiracoxib or a Cox-2 selective inhibitor. In some embodiments, the Cox-2 selective inhibitor is etoricoxib, celecoxib (SC-58635), 5-bromo-2-(4-fluorophenyl)-3-(4-(methylsulfonyl)phenyl)-thiophene (DUP-697), flosulide (CGP-28238), meloxicam, 6-methoxy-2 naphthylacetic acid (6-MNA), MK-966 (Vioxx), nabumetone (6-MNA prodrug), nimesulide, N-[2-(cyclohexyloxy)-4-nitrophenyl]-methanesulfonamide (NS-398), SC-5766, SC-58215, or 3-Formylamino-7-methylsulfonylamino-6-phenoxy-4H-1-benzopyran-1-one (T-614). In some embodiments, the anti-inflammatory agent is a corticosteroid. In some embodiments, corticosteroid is methyl prednisolone, prednisolone, dexamethasone, fluticasone propionate, 6α,9α-difluoro-17-[(2-furanylcarbonyl)oxy]-11β-hydroxy-16α-methyl-3-oxo-androsta-1,4-diene-17β-carbothioic acid S-fluoromethyl ester, 6α, 9α-difluoro-11β-hydroxy-16α-methyl-3-oxo-17.alpha.-propionyloxy-androsta-1,4-diene-17β-carbothioic acid S-(2-oxo-tetrahydro-furan-3S-yl) ester, beclomethasone esters, the 17-propionate ester or the 17,21-dipropionate ester, budesonide, flunisolide, mometasone esters, the furoate ester, triamcinolone acetonide, rofleponide, ciclesonide, butixocort propionate, RPR-106541, ST-126, fluticasone propionate, 6α,9α-difluoro-11β-hydroxy-16α-methyl-17α-[(-4-methyl-1,3-thiazole-5-carbonyl)oxy]-3-oxo-androsta-1,4-diene-17β-carbothioic acid S-fluoromethyl ester and 6α,9α-difluoro-17α-[(2-furanylcarbonyl)oxy]-11β-hydroxy-16α-methyl-3-oxo-androsta-1,4-diene-17β-carbothioic acid S-fluoromethyl ester, or 6α,9α-difluoro-17α-[(2-furanylcarbonyl)oxy]-11β-hydroxy-16α-methyl-3-oxo-androsta-1,4-diene-17β-carbothioic acid S-fluoromethyl ester. In some embodiments, the one or more other therapeutic agents comprise Colchicine or a prodrug thereof. In some embodiments, the one or more other therapeutic agents comprise an opioid agent. In some embodiments, the opioid agent is morphine, heroin, hydromorphone, oxymorphone, levorphanol, levallorphan, methadone, meperidine, fentanyl, cocaine, codeine, dihydrocodeine, oxycodone, hydrocodone, propoxyphene, nalmefene, nalorphine, naloxone, naltrexone, buprenorphine, butorphanol, nalbuphine or pentazocine. In some embodiments, the one or more other therapeutic agents comprise an IL-1 antagonist. In some embodiments, the IL-1 antagonist is Canakinumab (ACZ885) or Rilonacept (Arcalyst). In some embodiments, the one or more other therapeutic agents comprise IL-6 or a fragment thereof. In some embodiments, the one or more other therapeutic agents comprise a therapeutic agent selected from Table 2.

In one embodiment, the invention provides a method of treating hyperuricemia in a subject with gout comprising administering Tranilast to the subject in combination with one or more xanthine oxidase inhibitors or uricosuric agents. In some embodiments, the gout is refractory gout. In some embodiments, the Tranilast is administered in combination with allopurinol. The allopurinol can be administered in, for example, 100 mg or 300 mg doses, or in an optimized dose for combination with the composition of the present invention. For example, a lower dose of allopurinol may be effective when combined with Tranilast. In some embodiments, the Tranilast is administered in combination with febuxostat. The febuxostat can be administered in, for example, 40 mg or 80 mg or 120 mg doses, or in an optimized dose for combination with the composition of the present invention. For example, a lower dose of febuxostat may be effective when combined with Tranilast. The compositions of the present invention can be used in combination with any of the therapeutic agents listed in Table 2 in a similar manner. In other embodiments, a combination comprises Tranilast, a xanthane oxidase inhibitor, e.g., allopurinol or febuxostat, and a compound useful for the treatment of cardiovascular disorders, e.g., a statin. Any such combination therapy that addresses the numerous complications arising from hyperuricemia falls within the scope of the present invention.

In other embodiments, the methods of the present invention are used to treat effects associated with hyperuricemia and gout. For example, the methods of the present invention are used to treat pain associated with inflammation attributable to flares associate with gout attacks. In one embodiment, a method is provided for treating inflammatory pain associated with gout, comprising administering Tranilast or a pharmaceutically acceptable salt thereof. Optionally, one or more other therapeutic agents are administered in combination with Tranilast or a pharmaceutically acceptable salt thereof. In further embodiments, a method is provided for treating inflammatory pain associated with gout, comprising administering one or more pharmaceutical compositions of the invention or pharmaceutically acceptable salts thereof.

Pain may be assessed using a measurement index. Indices that are useful in the methods of the present invention for the measurement of pain associated with hyperuricemia and gout include a visual analog scale (VAS), a Likert scale, categorical pain scales, descriptors, and the AUSCAN index, each of which is well known in the art.

A visual analog scale (VAS) provides a measure of a one-dimensional quantity. A VAS generally utilizes a representation of distance, such as a picture of a line with hash marks drawn at regular distance intervals, e.g., ten 1-cm intervals. For example, a patient can be asked to rank a sensation of pain by choosing the spot on the line that best corresponds to the sensation of pain, where one end of the line corresponds to “no pain” (score of 0 cm) and the other end of the line corresponds to “unbearable pain” (score of 10 cm). This procedure provides a simple and rapid approach to obtaining quantitative information about how the patient is experiencing pain. VAS scales and their use are described, e.g., in U.S. Pat. Nos. 6,709,406 and 6,432,937.

A Likert scale similarly provides a measure of a one-dimensional quantity. Generally, a Likert scale has discrete integer values ranging from a low value (e.g., 0, meaning no pain) to a high value (e.g., 7, meaning extreme pain). A patient experiencing pain is asked to choose a number between the low value and the high value to represent the degree of pain experienced. Likert scales and their use are described, e.g., in U.S. Pat. Nos. 6,623,040 and 6,766,319.

The AUSCAN (Australian-Canadian hand arthritis) index employs a valid, reliable, and responsive patient self-reported questionnaire. In one instance, this questionnaire contains 15 questions within three dimensions (Pain, 5 questions; Stiffness, 1 question; and Physical function, 9 questions). An AUSCAN index may utilize, e.g., a Likert or a VAS scale.

Indices that are useful in the methods, compositions, and kits of the invention for the measurement of pain include the Pain Descriptor Scale (PDS), the Visual Analog Scale (VAS), the Verbal Descriptor Scales (VDS), the Numeric Pain Intensity Scale (NPIS), the Neuropathic Pain Scale (NPS), the Neuropathic Pain Symptom Inventory (NPSI), the Present Pain Inventory (PPI), the Geriatric Pain Measure (GPM), the McGill Pain Questionnaire (MPQ), mean pain intensity (Descriptor Differential Scale), numeric pain scale (NPS) global evaluation score (GES) the Short-Form McGill Pain Questionnaire, the Minnesota Multiphasic Personality Inventory, the Pain Profile and Multidimensional Pain Inventory, the Child Heath Questionnaire, and the Child Assessment Questionnaire.

The methods of the present invention are further useful for the treatment of inflammation and immune-related disorders. The administration of conventional urate lowering therapies leads to uric acid crystal remodeling that may result in inflammatory attacks and increase in painful flares, e.g., gout flares, during treatment. Moreover, patients suffering from gout suffer from chronic low level inflammation and the presence of inflammatory immune markers even in the absence of gouty attacks, as described herein. In some embodiments, the compositions and methods of the present invention provide anti-inflammatory and immune modulatory capabilities and are useful for reducing serum uric acid levels and concomitantly treating one or more of inflammation, inflammatory immune response, and pain associated therewith.

As described herein, a number of conditions are associated with hyperuricemia in addition to gout. These include a number of cardiovascular and renal complications. In some embodiments, the methods of the present invention are used to treat a renal disorder in a subject in need thereof. Renal disorders that can be treated with the methods of the invention include, but are not limited to, urinary lithiasis, hyperuricemic nephropathy, acute uric acid nephropathy, microalbuminuria, renal dysfunction, impaired glomerular filtration rate, and nephrolithiasis. In some embodiments, the methods of the present invention are used to treat kidney stones. The kidney stones may result directly from the deposition of uric acid, but may also result from the deposition of other materials, e.g., calcium oxalate or calcium phosphate, as sometimes observed in patients with hyperuricemia. In some embodiments, the methods of the present invention facilitate serum uric acid reduction in subjects suffering from renal insufficiency or chronic kidney disease. In some embodiments, treatments comprise administering a combination of a compound of the invention or pharmaceutically acceptable salt thereof and one or more therapeutic agents known to treat renal or urological disorders, including, but not limited to, a NO donor, a calcium channel blocker, a cholinergic modulator, an alpha-adrenergic receptor antagonist, a beta-adrenergic receptor agonist, a phosphodiesterase inhibitor, a cAMP-dependent protein kinase activator, a cAMP mimetic, a superoxide scavenger, a potassium channel activator, an estrogen-like compound, a testosterone-like compound, a benzodiazepine, an adrenergic nerve inhibitor, an antidiarrheal agent, a HMG-CoA reductase inhibitor, a smooth muscle relaxant, a adenosine receptor modulator, an adenylyl cyclase activator, an endothelin receptor antagonist, a bisphosphonate, a cGMP-dependent protein kinase activator, a cGMP mimetic, an alpha adrenergic blocking agent, Flomax, Uroxatral, terazosin, doxazosin, a nonsteroidal anti-inflammatories, an opioid, codeine, hydrocodone, thiazide, potassium citrate, magnesium citrate, allopurinol, or calgranulin.

In some embodiments, the methods of the invention are used to treat a cardiovascular disorder in a subject. Cardiovascular disorders that can be treated with the methods of the invention include, but are not limited to, hypertension, myocardial infraction, metabolic syndrome, ischemic cardiac disease, coronary artery disease, cerebrovascular disease, vascular dementia, preeclampsia, heart disease, stroke, atherogenesis, thrombogenesis, atheroscleorsis, inflammatory disease or peripheral, carotid, or coronary vascular disease. The methods are useful for treating or preventing any disorder associated with hyperuricemia, e.g., metabolic syndrome, hyperlipidemia, insulin resistance, diabetes, and adverse effects of obesity. In some embodiments, treatments comprise administering a combination of a compound of the invention or pharmaceutically acceptable salt thereof and one or more therapeutic agents known to treat a cardiovascular disorder, diabetes, or obesity, or complications thereof, including, but not limited to, glitazone, troglitazone, rosiglitazone (Avandia), pioglitazone, a sulphonylurea, gliquidone, tolbutamide, glimepride, chlorpropamide, glipizide, glyburide, acetohexamide, meglitinide, repaglinide, nateglinide, metformin, an endothelin receptor antagonist, bosentan, darusentan, enrasentan, tezosentan, atrasentan, ambrisentan sitaxsentan, a smooth muscle relaxant, a PDE5 inhibitor, minoxidil, an angiotensin converting enzyme (ACE) inhibitor, captopril, enalapril, lisinopril, fosinopril, perindopril, quinapril, trandolapril, benazepril, ramipril, a angiotensin II receptor blocker, irbesartan, losartan, valsartan, eprosartan, olmesartan, candesartan, telmisartan, a beta blocker, atenolol, metoprolol, nadolol, bisoprolol, pindolol, acebutolol, betaxolol, propranolol, a diuretic, thiazide, hydrochlorothiazide, furosemide, torsemide, metolazone, a calcium channel blocker, amlodipine, felodipine, nisoldipine, nifedipine, verapamil, diltiazem, a alpha receptor blocker, doxazosin, terazosin, alfuzosin, tamsulosin, a central alpha agonist, clonidine, a statin, atovastatin, fluvastatin, lovastatin, pravastatin, rosuvastatin calcium, simvastatin, nicotinic acid, a fibrate, gemfibrozil, fenofibrate, bezafibrate, ciprofibrate, a bile acid sequestrant, cholestyramine, colestipol, a cholesterol absorption inhibitor, a COX-1 inhibitor, aspirin, a NSAID, or a COX-2 inhibitor.

In some embodiments, the methods can further comprise measuring serum uric acid levels in the subject before and after the administering, wherein a decrease in serum uric acid levels after the administering indicates an effective treatment. For example, a decrease below about 6 mg/dL indicates lack of hyperuricemia. But any decrease might be beneficial to the patient, e.g., by lowering serum uric acid level below a level where uric acid crystals form. Other diagnostic approaches can be used with the present invention to indicate a beneficial treatment. These include, without limitation, reduction of uric acid crystals as determined by aspiration, visible reduction of tophi, reduced or eliminated symptoms of gout, e.g., reduced inflammation or pain, as decreased uric acid in urine, as well as imaging of uric acid crystal burden.

### (b) Methods of Dosing and Treatment Regimens

One or more active ingredients are optionally used in the preparation of medicaments for the prophylactic and/or therapeutic treatment of hyperuricemic conditions (e.g., gout) or conditions that would benefit, at least in part, from amelioration. In addition, a method for treating any of the diseases or conditions described herein in a subject in need of such treatment, involves administration of pharmaceutical compositions containing one or more active ingredients as described herein, or a pharmaceutically acceptable salt, pharmaceutically acceptable N-oxide, pharmaceutically active metabolite, pharmaceutically acceptable prodrug, or pharmaceutically acceptable solvate thereof, in therapeutically effective amounts to said subject.

In the case wherein the patient's condition does not improve, upon the doctor's discretion the administration of one or more active ingredients are optionally administered chronically, that is, for an extended period of time, including throughout the duration of the patient's life in order to ameliorate or otherwise control or limit the symptoms of the patient's disease or condition.

In the case wherein the patient's status does improve, upon the doctor's discretion the administration of one or more active ingredients are optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the symptoms, to a level at which the improved disease, disorder or condition is retained. In some embodiments, patients require intermittent treatment on a long-term basis upon any recurrence of symptoms.

In some embodiments, the pharmaceutical composition described herein is in unit dosage forms suitable for single administration of precise dosages, e.g., about 0.01 mg, 0.1 mg, 0.5 mg, 1 mg, 5 mg, 10 mg, 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1500 mg, or up to 2000 mg. In unit dosage form, the formulation is divided into unit doses containing appropriate quantities of the one or more active ingredients. In some embodiments, the unit dosage is in the form of a package containing discrete quantities of the formulation. Non-limiting examples are packaged tablets or capsules, and powders in vials or ampoules. In some embodiments, aqueous suspension compositions are packaged in single-dose non-reclosable containers. Alternatively, multiple-dose reclosable containers are used, in which case it is typical to include a preservative in the composition. By way of example only, formulations for parenteral injection are presented in unit dosage form, which include, but are not limited to ampoules, or in multi dose containers, with an added preservative.

The active ingredients and combinations thereof disclosed herein are contemplated to exhibit therapeutic activity when administered in an amount which can depend on the particular case. The variation in amount can depend, for example, on the human or animal and the active ingredients chosen. A broad range of doses can be applicable. Considering a subject, for example, from about 0.01 mg to about 2000 mg of a modulatory agent or active ingredient may be administered per day. For example, compounds of formula (I), formula (II), Tranilast or pharmaceutically acceptable salts thereof, can be administered from about 0.1 mg, 0.5 mg, 1 mg, 5 mg, 10 mg, 50 mg, 100 mg, 150 mg, 200 mg, 250 mg, 300 mg, 350 mg, 400 mg, 450 mg, 500 mg, 600 mg, 700 mg, 800 mg, 900 mg, 1000 mg to about 2000 mg of Tranilast per day. The daily dosages appropriate for one or more active ingredients can be from about 0.01 mg, 0.1 mg, 0.5 mg, 1 mg, 5 mg, 10 mg, 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1500 mg, or up to 2000 mg per day. The term “about” in the context of a particular measurement means 20% upward or downward of a number.

An indicated daily dosage in a larger mammal, including, but not limited to, humans, can be in the range from about 0.5 mg to about 5000 mg, conveniently administered in divided doses, including, but not limited to, up to four times a day or in extended release form. Dosage regimes may be adjusted to provide the optimum therapeutic response. For example, several divided doses may be administered daily, weekly, monthly or other at suitable time intervals or the dose may be proportionally reduced as indicated by the exigencies of the situation. Suitable unit dosage forms for oral administration can include from about 1 to 5000 mg active ingredient. The foregoing ranges are merely suggestive, as the number of variables in regard to an individual treatment regime is large, and considerable excursions from these recommended values are not uncommon. Such dosages are optionally altered depending on a number of variables, not limited to the activity of the one or more active ingredients used, the disease or condition to be treated, the mode of administration, the requirements of the individual subject, the severity of the disease or condition being treated, and the judgment of the practitioner.

One of skill in the art will appreciate that the dose and dosing regimen may be adjusted when therapeutic agents are used in combination. For example, a composition provided by the present invention, e.g., Tranilast or a pharmaceutically acceptable salt thereof, may be used in combination with a xanthane oxidase inhibitor, e.g., allopurinol or febuxostat. When such combinations are used, the dose of one or more of the agents may be reduced to a level below the level required for a desired efficacy when the one or more agents are used alone. Similarly, the dosing regimen may be modified, e.g., to synchronize the dosing of the one or more therapeutic agents to facilitate improved patient ease of use and compliance. Alternately, the dosing regimen of the one or more therapeutic agents can be sequential, e.g., to reduce the combined load of the agents at a given time.

Dose titration or dose escalation protocols may be employed to determine the proper or optimal dose to administer to a subject. For example, dose titration or escalation studies may select for doses that improve efficacy or tolerability. Dose titration or escalation allows for the gradual adjusting of the dose administered until the desired effect is achieved. Dose titration gradually decreased the dosage administered while dose escalation gradually increases the dose administered. Methods of dose titration and escalation are well known in the art. As a non-limiting example, a mammal may be administered 10 mg/day Tranilast every day and measured for serum uric acid levels on a daily basis. The dosage may be increased in increments of 5 mg/day on a weekly basis. The mammal may be monitored for a period of 12 weeks to find the desired dose.

Toxicity and therapeutic efficacy of such therapeutic regimens are optionally determined in cell cultures, experimental animals, or human studies, including, but not limited to, the determination of the LD50 (the dose lethal to 50% of the population) and the ED50 (the dose therapeutically effective in 50% of the population). The dose ratio between the toxic and therapeutic effects is the therapeutic index, which is expressed as the ratio between LD50 and ED50. Active ingredients exhibiting high therapeutic indices are preferred. The data obtained from cell culture assays and animal studies are optionally used in formulating a range of dosage for use in human. The dosage of such active ingredients lies preferably within a range of circulating concentrations that include the ED50 with minimal toxicity. The dosage optionally varies within this range depending upon the dosage form employed and the route of administration utilized.

### V. Kits

Kits are contemplated for use herein. In one embodiment, a kit comprises a first dosage form comprising a pharmaceutical composition of the present invention, e.g., containing Tranilast, in quantities sufficient to carry out the methods of the present invention, e.g., decreasing serum uric acid level, treating or preventing hyperuricemia, reducing pain or inflammation associated with hyperuricemia, treating or preventing gout, treating gouty symptoms, reducing the severity or number of gouty attacks, preventing, reducing or reversing uric acid crystal formation, treating a renal disorder, treating kidney stones, or treating a cardiovascular disorder. In some embodiments a kit is for a subject with a hyperuricemic disorder (e.g., gout) to use in the self-administration of the pharmaceutical composition, wherein the kit comprises a container housing a plurality of dosage forms and instructions for carrying out drug administration therewith. In one embodiment, a kit comprises a first dosage form comprising Tranilast in one or more of the forms identified above (e.g., a tablet, capsule, pill, delayed release formulation) and at least a second dosage form comprising one or more of the forms identified above, in quantities sufficient to carry out the methods of the present invention. The second dosage form, and any additional dosage forms (e.g., a third, fourth of fifth dosage form) can comprise any active ingredient disclosed herein for the treatment of a hyperuricemic disorder (e.g., gout). All dosage forms together can comprise a therapeutically effective amount of each compound for the treatment of a hyperuricemic disorder (e.g., gout). In some embodiments a kit is for a subject with a hyperuricemic disorder (e.g., gout) to use in the self-administration of at least one oral agent, wherein the kit comprises a container housing a plurality of said oral agents and instructions for carrying out drug administration therewith. At least one oral agent can comprise a combination of a therapeutically effective dose of Tranilast and a therapeutically effective dose of one or more of the following agents selected from the list consisting of Benzbromarone (URINORM), Probenecid, Allopurinol, Febuxostat (Uloric®), Bucolome, Cinchophan and Colchicine. In some embodiments a kit for use by a subject with a hyperuricemic disorder (e.g., gout) comprises at least one oral agent, a container housing a plurality of said oral agents and instructions for carrying out drug administration therewith, wherein said at least one oral agent comprises a combination of a therapeutically effective daily dose of Tranilast, or a pharmaceutically acceptable salt thereof and a daily dose of one or more of the following agents selected from the list consisting of Benzbromarone (URINORM), Probenecid, Allopurinol, Bucolome, Cinchophan and Colchicine. In some embodiments the one or more agents can be in distinct individual dosage forms or combined in a single dosage form or a combination of dosage forms thereof. In some embodiments, Tranilast, or a pharmaceutically acceptable salt thereof is in a distinct individual dosage form or combined in a single dosage form with one or more agents or a combination of dosage forms thereof.

### EXAMPLES

### Example 1

### Tranilast in Hyperuricemic Patients

The PRESTO (Prevention of Restenosis with Tranilast and its Outcomes) study was a multicenter study of ˜11,500 patients undergoing percutaneous transluminal coronary revascularization (PTCR) with or without stenting for single or multiple vessels over a 9-month period. The study compared the composite clinical event rate of death, myocardial infarction, or need for ischemia-driven target vessel revascularization in patients treated with Tranilast (300 and 450 mg twice daily) for 1 or 3 months versus placebo. Description of the study protocol and patient population can be found in Holmes et al., The PRESTO (Prevention of Restenosis with Tranilast and its Outcomes) protocol: A double-blind, placebo-controlled trial, Am Heart J 2000; 139:23-31; and Holmes et al., Results of Prevention of REStenosis with Tranilast and its Outcomes (PRESTO) Trial, Circulation. 2002; 106:1243, both of which are incorporated by reference herein in their entirety.

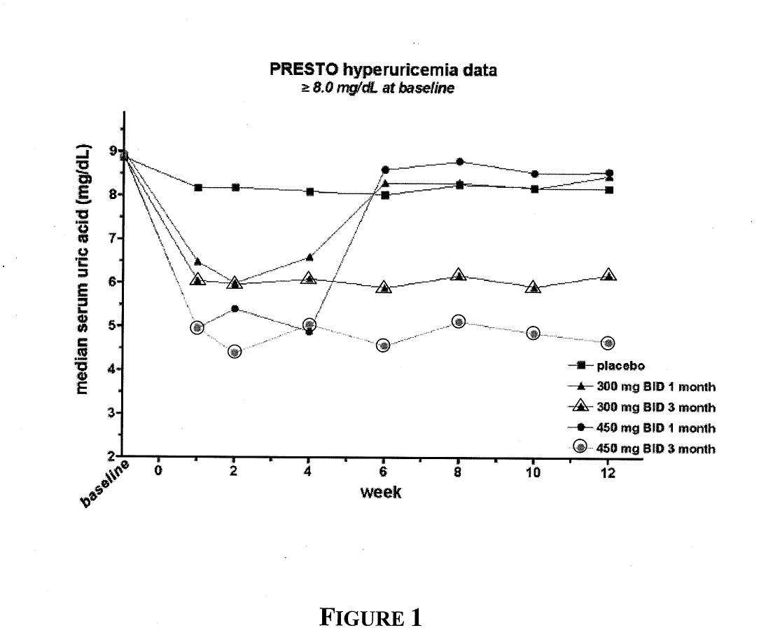

Gout was a contraindication for patient enrollment in the PRESTO trial. Nevertheless, 1100 patients enrolled in the trial were identified with baseline hyperuricemia defined as initial serum uric acid (sUA) levels greater than or equal to 7 mg/dL. Of these, approximately 300 participants had baseline serum uric acid levels greater than or equal to 8 mg/dL. In a prospective analysis of PRESTO data, serum uric acid levels were markedly decreased in patients treated with Tranilast. FIG. 1 shows the effects of Tranilast on uric acid levels in the hyperuricemic patients having uric acid baseline levels greater than or equal to 8 mg/dL. Data for FIG. 1 are shown in Table 3. Table 4 shows the demographic characteristics of patient population for FIG. 1 and Table 4. As indicated in FIG. 1 and Table 4, some patients were treated with placebo, some patients were treated with Tranilast for 4 weeks followed by placebo, and some patients were treated with Tranilast for 12 weeks.

TABLE 3
Change from Baseline in Uric Acid (mg/dL) over Time by Treatment Group

| | | | 1M TRAN | 1M TRAN | | |
|---|---|---|---|---|---|---|
| | | | 300 mg/ | 450 mg/ | 3M TRAN | |
| Visit | Statistic | 3M PLACEBO | 2M PLACEBO | 2M PLACEBO | 300 mg | 3M TRAN 450 mg |
| Baseline | N | 57 | 54 | 60 | 82 | 54 |
| | Mean (SD) | 8.86 (1.070) | 8.90 (0.949) | 8.82 (0.929) | 8.91 (1.446) | 8.81 (0.797) |
| | Median | 8.47 | 8.60 | 8.51 | 8.50 | 8.55 |
| | Min, Max | 8.02, 14.24 | 8.04, 12.80 | 8.07, 12.90 | 8.02, 19.38 | 8.07, 11.80 |
| Post-PTCR | N | 55 | 49 | 56 | 76 | 53 |
| | Mean (SD) | −0.80 (1.352) | −1.33 (1.220) | −2.00 (1.454) | −1.81 (1.966) | −2.16 (1.577) |
| | Median | −0.80 | −1.30 | −1.96 | −1.58 | −1.90 |
| | Min, Max | −8.44, 2.75 | −4.54, 2.03 | −5.21, 0.70 | −14.79, 1.90 | −7.34, 1.70 |
| Week 1 | N | 51 | 51 | 54 | 72 | 51 |
| | Mean (SD) | −0.95 (1.417) | −2.31 (1.839) | −3.66 (1.902) | −2.96 (2.394) | −3.84 (1.839) |
| | Median | −0.70 | −2.51 | −3.95 | −2.85 | −3.99 |
| | Min, Max | −8.56, 1.63 | −6.05, 2.70 | −8.00, 2.00 | −15.66, 3.14 | −8.79, 0.90 |
| Week 2 | N | 50 | 47 | 52 | 72 | 50 |
| | Mean (SD) | −0.90 (1.387) | −2.61 (2.149) | −3.43 (2.176) | −2.98 (2.224) | −4.05 (1.861) |
| | Median | −0.69 | −3.00 | −3.50 | −2.94 | −4.56 |
| | Min, Max | −7.77, 1.80 | −7.05, 1.80 | −8.40, 1.00 | −15.09, 2.30 | −8.72, 0.92 |
| Week 3 | N | 22 | 24 | 22 | 29 | 18 |
| | Mean (SD) | −0.93 (0.974) | −2.43 (2.143) | −4.19 (1.978) | −2.92 (1.946) | −2.84 (2.227) |
| | Median | −0.78 | −2.70 | −4.27 | −3.00 | −3.79 |
| | Min, Max | −3.90, 0.50 | −6.61, 3.50 | −7.90, −0.70 | −6.13, 1.92 | −5.79, 1.50 |
| Week 4 | N | 48 | 46 | 45 | 71 | 49 |
| | Mean (SD) | −0.94 (1.469) | −2.30 (1.953) | −3.52 (2.127) | −2.83 (2.212) | −3.70 (2.190) |
| | Median | −0.78 | −2.40 | −4.03 | −2.80 | −3.90 |
| | Min, Max | −8.29, 1.70 | −7.40, 2.60 | −7.65, 1.00 | −14.30, 1.98 | −8.40, 1.43 |
| Week 6 | N | 45 | 40 | 41 | 64 | 42 |
| | Mean (SD) | −0.84 (1.495) | −0.82 (1.416) | −0.29 (1.188) | −2.75 (2.755) | −3.89 (2.265) |
| | Median | −0.84 | −0.70 | −0.30 | −3.00 | −4.39 |
| | Min, Max | −8.24, 1.40 | −5.21, 2.40 | −3.20, 2.50 | −15.65, 8.50 | −8.69, 2.70 |
| Week 8 | N | 49 | 37 | 37 | 59 | 38 |
| | Mean (SD) | −0.93 (1.576) | −0.63 (1.371) | −0.49 (2.142) | −2.76 (2.451) | −3.65 (2.104) |
| | Median | −0.60 | −0.70 | −0.10 | −2.71 | −3.83 |
| | Min, Max | −9.10, 1.43 | −4.54, 1.55 | −9.70, 3.60 | −15.77, 1.70 | −7.16, 1.40 |
| Week 10 | N | 46 | 36 | 37 | 54 | 38 |
| | Mean (SD) | −0.98 (1.725) | −0.79 (1.195) | −0.46 (1.494) | −2.97 (2.490) | −3.73 (2.306) |
| | Median | −0.68 | −0.83 | −0.38 | −2.98 | −4.10 |
| | Min, Max | −8.64, 2.34 | −3.53, 2.10 | −4.40, 4.60 | −15.65, 2.30 | −8.60, 1.50 |
| Week 12 | N | 47 | 34 | 36 | 53 | 36 |
| | Mean (SD) | −0.88 (1.535) | −0.75 (1.500) | −0.56 (1.298) | −2.74 (2.634) | −3.79 (2.065) |
| | Median | −0.70 | −0.52 | −0.34 | −2.69 | −4.30 |
| | Min, Max | −8.66, 1.30 | −4.90, 1.50 | −3.50, 2.70 | −16.02, 3.60 | −8.12, 0.50 |
| Follow-Up | N | 17 | 18 | 21 | 26 | 17 |
| | Mean (SD) | −1.30 (2.413) | −0.64 (1.351) | −0.53 (1.249) | −0.00 (1.519) | −0.67 (1.277) |
| | Median | −0.70 | −0.86 | −0.40 | −0.13 | −0.70 |
| | Min, Max | −9.10, 1.00 | −3.30, 2.00 | −4.40, 1.20 | −3.00, 3.04 | −2.90, 1.80 |

TABLE 4
Demographic Characteristics

| | | | | 1M TRAN | | 1M TRAN | | | | | | | |
|---|---|---|---|---|---|---|---|---|---|---|---|---|---|
| | | | | 300 mg/ | | 450 mg/ | | | | | | | |
| | | 3M | | 2M | | 2M | | 3M TRAN | | 3M TRAN | | | |
| | | PLACEBO | | PLACEBO | | PLACEBO | | 300 mg | | 450 mg | | Total | |
| | | (N = 64) | | (N = 57) | | (N = 65) | | (N = 84) | | (N = 59) | | (N = 329) | |
| Gender | Male | 51 | (79.7%) | 43 | (75.4%) | 54 | (83.1%) | 68 | (81.0%) | 48 | (81.4%) | 264 | (80.2%) |
| | Female | 13 | (20.3%) | 14 | (24.6%) | 11 | (16.9%) | 16 | (19.0%) | 11 | (18.6%) | 65 | (19.8%) |
| Age (years) | <45 | 7 | (10.9%) | 5 | (8.8%) | 7 | (10.8%) | 5 | (6.0%) | 4 | (6.8%) | 28 | (8.5%) |
| | 45-64 | 27 | (42.2%) | 31 | (54.4%) | 25 | (38.5%) | 42 | (50.0%) | 38 | (64.4%) | 163 | (49.5%) |
| | 65-69 | 11 | (17.2%) | 9 | (15.8%) | 13 | (20.0%) | 14 | (16.7%) | 8 | (13.6%) | 55 | (16.7%) |
| | 70-74 | 12 | (18.8%) | 7 | (12.3%) | 8 | (12.3%) | 11 | (13.1%) | 8 | (13.6%) | 46 | (14.0%) |
| | >=75 | 7 | (10.9%) | 5 | (8.8%) | 12 | (18.5%) | 12 | (14.3%) | 1 | (1.7%) | 37 | (11.2%) |
| Race | Caucasian | 62 | (96.9%) | 46 | (80.7%) | 57 | (87.7%) | 78 | (92.9%) | 58 | (98.3%) | 301 | (91.5%) |
| | Black | 0 | (0.0%) | 6 | (10.5%) | 4 | (6.2%) | 4 | (4.8%) | 1 | (1.7%) | 15 | (4.6%) |
| | Oriental | 0 | (0.0%) | 0 | (0.0%) | 0 | (0.0%) | 0 | (0.0%) | 0 | (0.0%) | 0 | (0.0%) |
| | Other | 2 | (3.1%) | 5 | (8.8%) | 4 | (6.2%) | 2 | (2.4%) | 0 | (0.0%) | 13 | (4.0%) |

| | | | | | | | |
|---|---|---|---|---|---|---|---|
| Age (years) | N | 64 | 57 | 65 | 84 | 59 | 329 |
| | MEAN | 60.656 | 60.561 | 62.523 | 61.845 | 58.475 | 60.921 |
| | MEDIAN | 62 | 62 | 65 | 62 | 59 | 62 |
| | STD | 11.680 | 11.220 | 11.937 | 11.175 | 9.183 | 11.129 |
| | Q1 | 52 | 51 | 57 | 54 | 50 | 52 |
| | Q3 | 70 | 69 | 72 | 71 | 66 | 70 |
| | MIN | 36 | 40 | 32 | 33 | 41 | 32 |
| | MAX | 82 | 84 | 80 | 83 | 75 | 84 |
| Weight (kg) | N | 64 | 57 | 65 | 84 | 59 | 329 |
| | MEAN | 91.233 | 94.610 | 90.729 | 90.376 | 93.227 | 91.857 |
| | MEDIAN | 90 | 92 | 90 | 88 | 93 | 90 |
| | STD | 16.965 | 21.202 | 16.658 | 18.576 | 17.107 | 18.105 |
| | Q1 | 77 | 81 | 80 | 77 | 80 | 79 |
| | Q3 | 101 | 101 | 101 | 103 | 105 | 102 |
| | MIN | 50 | 64 | 53 | 55 | 51 | 50 |
| | MAX | 129 | 161 | 134 | 153 | 136 | 161 |
| Height (cm) | N | 64 | 57 | 65 | 83 | 59 | 328 |
| | MEAN | 170.713 | 170.476 | 171.647 | 170.844 | 172.617 | 171.233 |
| | MEDIAN | 171 | 170 | 172 | 171 | 174 | 172 |
| | STD | 8.700 | 8.121 | 8.485 | 9.226 | 9.353 | 8.798 |
| | Q1 | 165 | 165 | 165 | 165 | 168 | 165 |
| | Q3 | 178 | 176 | 180 | 180 | 180 | 178 |
| | MIN | 152 | 150 | 152 | 147 | 150 | 147 |
| | MAX | 186 | 188 | 191 | 185 | 189 | 191 |

### Example 2

### Study A3006GT

Study A3006GT was a phase 2, randomized, double-blind, 3-period, 3-treatment crossover trial. The primary objective was to compare the effects of three treatments on serum uric acid (sUA) levels in healthy subjects with a screening sUA level of at least 7.1 mg/dL. The three treatments were: 300 mg QD tranilast and 40 mg QD febuxostat (C); 300 mg QD tranilast alone (T); 40 mg QD febuxostat alone (F).

The study was conducted in 23 subjects. For each study period for which data were obtained, Table 9 displays the baseline sUA level for each subject, as well as the change and percent change from baseline at the end of the 1-week treatment period. With two exceptions, every subject provided data for all three treatments. Subject 1001 completed only the first two periods of the study (C and F). Although subject 1024 completed all three periods, this subject did not receive the assigned treatment in period 3 (F). Therefore, the period 3 data for subject 1024 are excluded from the analyses presented here.

Table 5 provides basic summary statistics (mean, standard deviation [SD], median, minimum, maximum) for the change from baseline and the percent change from baseline for each of the three treatments. For both the change from baseline and the percent change from baseline, the mean and median change in the combination group (C) is larger than the change in either group F or group T.

TABLE 5
Summary Statistics for sUA Change and Percent Change
from Baseline, by Treatment

| Variable | Treatment | n | Mean | SD | Median | Min | Max |
|---|---|---|---|---|---|---|---|
| Change | C | 23 | −5.09 | 0.92 | −5.00 | −7.30 | −3.60 |
| | F | 22 | −2.75 | 0.84 | −2.85 | −3.80 | −0.70 |
| | T | 22 | −1.60 | 0.70 | −1.40 | −3.50 | −0.90 |
| % Change | C | 23 | −66.28 | 9.49 | −65.06 | −86.30 | −51.32 |
| | F | 22 | −35.32 | 9.60 | −36.86 | −48.68 | −9.21 |
| | T | 22 | −21.20 | 9.19 | −18.61 | −47.95 | −9.47 |

### Methodology

Two statistical approaches were used. Both approaches were carried out using both the change from baseline and the percent change from baseline as the endpoint of interest. The following description refers generically to “change”, but applies to both change from baseline and percent change from baseline.

Two endpoints were considered: change from baseline to day 7 in sUA; and percent change from baseline to day 7 in sUA. For each endpoint, an analysis of variance (ANOVA) model for a three-period crossover trial was used. The model included effects for sequence, subject within sequence, treatment, and period. The sequence effect was tested using the subject within sequence mean square as the error term. Two pairwise comparisons between treatments were tested: C−F and C−T. In addition, the two-sided 95% confidence interval for each of these differences was computed.

These analyses were completed in three subsets of the data: all nonmissing data; all periods where the baseline sUA was greater than or equal to 6.8 mg/dL; all periods where the baseline sUA was greater than 6.8 mg/dL.

In addition, each of these three subsets was analyzed using all available data (regardless as to whether a subject provided data for all three periods of the study) and additionally using only those subjects who provided complete data for all three periods.

### Assessment of Subject-Specific Differences

For each subject for which results were available from all three treatments, the difference between the change for C and the sum of the changes for F and T was computed. Although the true magnitude of the difference between these two values is not known, a conservative null hypothesis is that this difference should be equally likely to be greater than or less than zero. The sign test and the Wilcoxon signed rank test were used to test this null hypothesis.

These analyses were carried out in three subsets of subjects: all subjects with values for all three treatments; subjects whose baseline sUA for all three treatments was greater than or equal to 6.8 mg/dL; and subjects whose baseline sUA for all three treatments was greater than 6.8 mg/dL.

### Assessment of Group Mean Differences

Using the change for each subject as the dependent variable, an analysis of variance (ANOVA) model for a three-period crossover trial was fit. This model included effects for sequence, subject within sequence, treatment, and period. Using the least squares estimates of the mean changes for each treatment, along with the 3×3 covariance matrix of these three estimates, the mean and SD of the difference between C and F+T was estimated, as follows.

Let x denote the 3×1 column vector of the estimated mean changes, ordered as (C F T), and let V denote the estimated variance-covariance matrix of x. The quantity of interest (difference between C and F+T) is then estimated by y=a*x, where a is the 1×3 row vector (1 −1 −1). The variance of y is estimated by aVa′, where a′ denotes the transpose of a. Based on the estimated mean and its standard deviation (square root of the variance), the p-value from the two-sided test that the mean difference is equal to zero and the two-sided 95% confidence interval for this difference was then computed using the appropriate t distribution. If the upper limit of this confidence interval is less than zero, then the mean change for C is assessed as being greater than the sum of the mean changes for F and T.

These analyses were completed in three subsets of the data: all nonmissing data; all periods where the baseline sUA was greater than or equal to 6.8 mg/dl; and all periods where the baseline sUA was greater than 6.8 mg/dL.

In addition, each of these three subsets was analyzed using all available data (regardless as to whether a subject provided data for all three periods of the study) and also using only those subjects who provided complete data for all three periods.

### Results

Table 6 displays the results for the comparisons between C and F, and between C and T.

Comparisons between C and F, and between C and T

| | | | | Difference between Combination | | | | |
|---|---|---|---|---|---|---|---|---|
| | | | | Treatment and Individual Treatment | | | | |
| | | Baseline | | | | | 95% CI | |
| Variable | Data | Subset | Comparison | Est. | SE | p-value | Lower | Upper |
| Change | All | All | C-F | −2.34 | 0.20 | <.0001 | −2.75 | −1.93 |
| | | | C-T | −3.48 | 0.20 | <.0001 | −3.89 | −3.07 |
| | | 6.8+ | C-F | −2.45 | 0.25 | <.0001 | −2.96 | −1.95 |
| | | | C-T | −3.52 | 0.24 | <.0001 | −4.01 | −3.02 |
| | | >6.8 | C-F | −2.51 | 0.26 | <.0001 | −3.04 | −1.99 |
| | | | C-T | −3.56 | 0.25 | <.0001 | −4.08 | −3.05 |
| | Complete | All | C-F | −2.29 | 0.21 | <.0001 | −2.72 | −1.87 |
| | | | C-T | −3.48 | 0.21 | <.0001 | −3.91 | −3.05 |
| | | 6.8+ | C-F | −2.40 | 0.27 | <.0001 | −2.94 | −1.85 |
| | | | C-T | −3.51 | 0.26 | <.0001 | −4.04 | −2.98 |
| | | >6.8 | C-F | −2.39 | 0.26 | <.0001 | −2.93 | −1.84 |
| | | | C-T | −3.61 | 0.25 | <.0001 | −4.14 | −3.09 |
| % Change | All | All | C-F | −31.07 | 2.32 | <.0001 | −35.77 | −26.37 |
| | | | C-T | −44.98 | 2.32 | <.0001 | −49.68 | −40.28 |
| | | 6.8+ | C-F | −32.02 | 2.84 | <.0001 | −37.82 | −26.21 |
| | | | C-T | −44.13 | 2.80 | <.0001 | −49.84 | −38.41 |
| | | >6.8 | C-F | −32.36 | 2.98 | <.0001 | −38.46 | −26.26 |
| | | | C-T | −44.41 | 2.91 | <.0001 | −50.36 | −38.46 |
| | Complete | All | C-F | −30.48 | 2.40 | <.0001 | −35.35 | −25.62 |
| | | | C-T | −45.08 | 2.40 | <.0001 | −49.95 | −40.22 |
| | | 6.8+ | C-F | −31.19 | 3.02 | <.0001 | −37.37 | −25.01 |
| | | | C-T | −44.10 | 2.94 | <.0001 | −50.13 | −38.07 |
| | | >6.8 | C-F | −30.54 | 2.96 | <.0001 | −36.63 | −24.46 |
| | | | C-T | −45.08 | 2.84 | <.0001 | −50.91 | −39.24 |

For both the change from baseline and the percent change from baseline, all differences between C and F, and between C and T, are negative (indicating a larger reduction in sUA with the combination treatment than with an individual treatment). In addition, all differences are highly statistically significantly different from zero (p<0.0001) and, consequently, the 95% confidence intervals for the differences all exclude zero.

For change from baseline, the mean differences between C and F are always to be in the range from −2.29 to −2.45 mg/dL. The mean differences between C and T are larger, and range from −3.48 to −3.61 mg/dL.

For percent change from baseline, the mean differences between C and F are estimated to be in the range from −30% to −32%. The mean differences between C and T are again larger, and range from −44% to −45%.

### Assessment of Subject-Specific Differences

For the change from baseline, data were available from all three treatments for 21 of 23 subjects. The difference between C and F+T was negative (indicating a greater decrease with C than the sum of the decreases for F+T) for 19 subjects, equal to zero for 1 subject, and positive for 1 subject. This distribution of differences is highly statistically significant (p=0.0015 from the sign test and p=0.0006 from the Wilcoxon signed rank test).

For the percent change from baseline, data were also available from all three treatments for 21 of 23 subjects. The difference between C and F+T was negative (indicating a greater decrease with C than the sum of the decreases for F+T) for 19 subjects and positive for 2 subjects. This distribution of differences is also highly statistically significant (p=0.0002 from the sign test and p=0.0003 from the Wilcoxon signed rank test).

These analyses were repeated in two subgroups: subjects whose baseline sUA for all three treatments was greater than or equal to 6.8 mg/dL; and subjects whose baseline sUA for all three treatments was greater than 6.8 mg/dL.

Table 7 displays the resulting p-values from the sign test and signed rank test for the change and percent change from baseline. In all analyses, the null hypothesis of equality of C and F+T is rejected.

TABLE 7
Tests of Subject-Specific Differences in Subgroups Defined by
Baseline sUA Level

| | | | p-value | |
|---|---|---|---|---|
| | | Number | | Wilcoxon |
| | Baseline | of | Sign | Signed Rank |
| Variable | Subgroup | Subjects | Test | Test |
| Change | 6.8 mg/dL or greater | 16 | 0.0005 | 0.0013 |
| % Change | 6.8 mg/dL or greater | 16 | 0.0005 | 0.0010 |

### Assessment of Group Mean Differences

For each of the three baseline subsets (all data, baseline sUA greater than or equal to 6.8 mg/dL, baseline sUA greater than 6.8 mg/dL), Table 8 displays results based on the ANOVA models for both endpoints (change, percent change) using all available data and using only the data from subjects with complete data for all three periods of the study. For each analysis, the number of subjects and the total number of data values (number of observations) are first displayed. The estimated mean difference between C and F+T and the SD of this difference are also provided, as well as the p-value from the test of the null hypothesis that the mean difference between C and F+T is equal to zero. Finally, the 95% confidence interval (CI) for the difference is reported.

TABLE 8
Estimates and 95% Confidence Intervals for the Mean Difference between C and F + T

| | | | | | Difference Between C and F + T | | | | |
|---|---|---|---|---|---|---|---|---|---|
| | | Baseline | Sample Size | | | | | 95% CI | |
| Variable | Data | Subset | Subj. | Obs. | Est. | SD | p | Lower | Upper |
| Change | All | All | 23 | 67 | −0.76 | 0.25 | 0.004 | −1.26 | −0.25 |
| | | 6.8+ | 22 | 56 | −0.78 | 0.32 | 0.020 | −1.42 | −0.13 |
| | | >6.8 | 21 | 54 | −0.83 | 0.32 | 0.014 | −1.49 | −0.18 |
| | Complete | All | 21 | 63 | −0.76 | 0.26 | 0.005 | −1.28 | −0.24 |
| | | 6.8+ | 16 | 48 | −0.76 | 0.32 | 0.026 | −1.42 | −0.10 |
| | | >6.8 | 15 | 45 | −0.77 | 0.32 | 0.024 | −1.43 | −0.11 |
| % Change | All | All | 23 | 67 | −10.03 | 2.87 | 0.001 | −15.83 | −4.23 |
| | | 6.8+ | 22 | 56 | −9.53 | 3.64 | 0.014 | −16.96 | −2.10 |
| | | >6.8 | 21 | 54 | −10.14 | 3.72 | 0.011 | −17.74 | −2.54 |
| | Complete | All | 21 | 63 | −9.90 | 2.94 | 0.002 | −15.86 | −3.94 |
| | | 6.8+ | 16 | 48 | −10.55 | 3.68 | 0.008 | −18.07 | −3.02 |
| | | >6.8 | 15 | 45 | −10.48 | 3.58 | 0.007 | −17.84 | −3.12 |

In every case, the point estimates of the mean difference between C and F+T are negative, indicating that the mean change (or percent change) due to C is larger than the sum of the mean changes for F+T. In addition, in every analysis the 95% confidence interval for the estimated mean difference between C and F+T does not include zero, indicating that this difference is statistically significantly different from zero.

TABLE 9
Listing of sUA Results from Individual Subjects

| | | | | | Percent |
|---|---|---|---|---|---|
| ID | Period | Treatment | Baseline | Change | Change |
| 1001 | 1 | C | 7.7 | −5.8 | −75.32 |
| | 2 | F | 8.0 | −2.6 | −32.50 |
| 1002 | 1 | T | 7.8 | −1.7 | −21.79 |
| | 2 | C | 8.1 | −4.4 | −54.32 |
| | 3 | F | 7.6 | −0.7 | −9.21 |
| 1003 | 1 | T | 8.0 | −1.4 | −17.50 |
| | 2 | F | 8.1 | −2.9 | −35.80 |
| | 3 | C | 7.2 | −4.5 | −62.50 |
| 1004 | 1 | F | 7.5 | −2.1 | −28.00 |
| | 2 | T | 7.4 | −1.1 | −14.86 |
| | 3 | C | 7.6 | −3.9 | −51.32 |
| 1005 | 1 | C | 6.8 | −3.9 | −57.35 |
| | 2 | T | 6.9 | −2.1 | −30.43 |
| | 3 | F | 7.1 | −1.2 | −16.90 |
| 1006 | 1 | F | 7.9 | −1.8 | −22.78 |
| | 2 | C | 7.9 | −4.4 | −55.70 |
| | 3 | T | 7.9 | −2.2 | −27.85 |
| 1007 | 1 | C | 6.4 | −4.0 | −62.50 |
| | 2 | T | 6.7 | −1.1 | −16.42 |
| | 3 | F | 7.0 | −2.9 | −41.43 |
| 1009 | 1 | C | 6.1 | −3.6 | −59.02 |
| | 2 | F | 6.5 | −2.8 | −43.08 |
| | 3 | T | 6.3 | −1.7 | −26.98 |
| 1010 | 1 | F | 8.7 | −3.5 | −40.23 |
| | 2 | C | 8.5 | −6.4 | −75.29 |
| | 3 | T | 8.6 | −1.9 | −22.09 |
| 1011 | 1 | T | 6.6 | −0.9 | −13.64 |
| | 2 | C | 7.6 | −5.1 | −67.11 |
| | 3 | F | 6.7 | −2.4 | −35.82 |
| 1012 | 1 | F | 10.1 | −3.7 | −36.63 |
| | 2 | T | 9.5 | −0.9 | −9.47 |
| | 3 | C | 9.7 | −5.1 | −52.58 |
| 1013 | 1 | F | 6.5 | −2.7 | −41.54 |
| | 2 | T | 6.5 | −1.7 | −26.15 |
| | 3 | C | 7.1 | −5.3 | −74.65 |
| 1014 | 1 | T | 7.5 | −1.2 | −16.00 |
| | 2 | C | 7.7 | −6.0 | −77.92 |
| | 3 | F | 7.7 | −3.6 | −46.75 |
| 1015 | 1 | T | 7.7 | −1.1 | −14.29 |
| | 2 | F | 7.4 | −2.1 | −28.38 |
| | 3 | C | 7.6 | −4.5 | −59.21 |
| 1016 | 1 | C | 8.3 | −5.4 | −65.06 |
| | 2 | F | 8.9 | −3.3 | −37.08 |
| | 3 | T | 8.4 | −1.2 | −14.29 |
| 1017 | 1 | C | 9.1 | −6.2 | −68.13 |
| | 2 | T | 9.2 | −1.0 | −10.87 |
| | 3 | F | 8.8 | −3.8 | −43.18 |
| 1018 | 1 | F | 7.0 | −2.2 | −31.43 |
| | 2 | C | 7.0 | −5.3 | −75.71 |
| | 3 | T | 7.3 | −2.2 | −30.14 |
| 1019 | 1 | C | 7.8 | −4.9 | −62.82 |
| | 2 | T | 8.3 | −1.2 | −14.46 |
| | 3 | F | 8.6 | −3.5 | −40.70 |
| 1020 | 1 | F | 9.2 | −3.6 | −39.13 |
| | 2 | T | 9.0 | −3.2 | −35.56 |
| | 3 | C | 9.4 | −7.3 | −77.66 |
| 1021 | 1 | F | 7.6 | −3.7 | −48.68 |
| | 2 | C | 7.3 | −6.3 | −86.30 |
| | 3 | T | 7.3 | −3.5 | −47.95 |
| 1024 | 1 | T | 7.8 | −1.6 | −20.51 |
| | 2 | C | 8.1 | −4.9 | −60.49 |
| | 3 | F | 8.0 | | |
| 1025 | 1 | F | 7.3 | −3.1 | −42.47 |
| | 2 | T | 7.1 | −1.4 | −19.72 |
| | 3 | C | 7.0 | −4.9 | −70.00 |
| 1026 | 1 | C | 6.8 | −5.0 | −73.53 |
| | 2 | T | 6.5 | −1.0 | −15.38 |
| | 3 | F | 6.5 | −2.3 | −35.38 |

### Example 3

### Study A3008GT

This is a Phase 2, randomized, double-blind, 3-period, 3-treatment, balanced crossover study in otherwise healthy subjects with documented hyperuricemia and a screening sUA level≧7.1 mg/dL to evaluate the effect of tranilast on allopurinol and oxipurinol PK and pharmacodynamics (PD) and to evaluate the effect of allopurinol on tranilast PK and PD.

The study utilizes 3 dosing periods with each subject receiving all 3 treatments orally: 300 mg QD tranilast alone (T), 300 mg QD allopurinol alone (A), and the combination of 300 mg QD tranilast and 300 mg QD allopurinol (C). Subjects are randomized in a 1:1:1:1:1:1 ratio to receive each of the 3 treatments in one of 6 possible sequences: TAC, TCA, ATC, ACT, CTA, or CAT. Each period is 14 days in duration with 7 consecutive days of active treatment (Days 1-7), followed by a 7-day drug-free washout interval (Days 8-14).

Subjects are screened for eligibility within 28 days before the start of dosing in Period 1. During each of the 3 periods, subjects check-in to the Clinical Study Unit (CSU) on the morning of Day −2 and will be domiciled until all study procedures are completed the morning of Day 8 (i.e., in the CSU for 9 full days and nights in each period, for a total of 27 full days and nights in the CSU to complete the study).

During Day −1 of each period, baseline PD testing (sUA and urinary uric acid [uUA]) are initiated. Active treatment will occur on Days 1±7 of each period. Blood samples for the determination of sUA are collected on each day of dosing.

Urine is collected over 24 hours, at timed intervals, to evaluate uric acid excretion and creatinine clearance at baseline of each period (Day −1), on the first day of dosing in each period (Day 1), and the last day of dosing in each period (Day 7). Blood samples for the determination of trough plasma levels of tranilast and/or allopurinol and its metabolite oxipurinol are obtained on Days 6, 7, and 8 of each dosing period. Complete plasma concentration versus time profiles for the key PK measurements are evaluated over the 24-hour interval after the last dose(s) of tranilast and/or allopurinol/oxipurinol in each period, with samples collected at 0, 0.5, 1, 1.5, 2, 3, 4, 5, 6, 8, 10, 12, 18, and 24 hours after the last dose of study drug on Day 7 of each period.

Safety laboratory testing (including liver and renal function assessments) and an evaluation of C-reactive protein (CRP), a marker of inflammation, are collected at screening, at baseline of each period (Day −1), the day after completion of dosing in each period (Day 8), and on Day 14 (±1 day) of Period 3. In addition, a biochemistry panel is tested on Days 3 and 5 to monitor the liver and renal function.

### Example 4

### Air Pouch Model 1

The objective of this study was to evaluate the anti-inflammatory affects of tranilast versus a clinically active treatment for gout, colchicine, as well as a clinically active non-steroidal antiinflammatory drug, indomethacin. This evaluation was carried out in male Sprague-Dawley rats in a rodent model of gout. The animals were injected subcutaneously with 20 ml of sterile air, followed three days later by a supplemental injection with 20 ml of sterile air. Six days after the initial sterile air injection, the rats were injected intravenously with Evan's Blue and pretreated for thirty minutes with either a subcutaneous injection of colchicine (1 mg/kg) or indomethacin (5 mg/kg) or oral administration with either 200 mg/kg or 400 mg/kg of tranilast. After the pretreatment period, the rats were injected with 150 mg of monosodium urate (MSU) crystals (10 mg·ml) into the air pouch. Four hours later, the air pouch was injected with 5 ml heparinized saline and the entire contents of the air pouch removed, recording the total volume. The air pouch contents were evaluated for plasma extravasation and total white blood cell (WBC) count.

On day 0 the rats were anesthetized in a biological cabinet, the nape of the neck was cleansed with 70% isopropanol followed by iodine tincture (VEDCO, lot L021976). Twenty ml sterile (0.22 μm, Fisher Scientific, Cat. 09-720-3, lot R5SN25683) air was injected subcutaneously using a 23 G×2 inch needle fixed to a 20 ml syringe. The rats were returned to routine housing. No adverse reactions were observed.

On day 3 the rats were anesthetized in a biological cabinet, the nape of the neck was cleansed with 70% isopropanol followed by iodine tincture (VEDCO, lot L021976). Twenty ml sterile (0.22 μm, Fisher Scientific, Cat. 09-720-3, lot R5SN25683) air was injected subcutaneously using a 23 G×2 inch needle fixed to a 20 ml syringe. The rats were returned to routine housing. No adverse reactions were observed.

On day 6 the rats were weighed (Mettler, Model PE3000, SN: F69474) and sorted into six treatment groups of ten animals each, based upon average weight. The animals were either dosed subcutaneously with 1 ml/kg colchicine or indomethacin, or orally with either 4 ml/kg or 8 ml/kg tranilast, or 8 ml/kg vehicle. Immediately after test material administration, the rats were intravenously injected with 2 ml/kg Evans Blue.

TABLE 10
Treatment Groups

| | | Dose | |
|---|---|---|---|
| Group | Treatment | (mg/kg) | +MSU |
| 1 | Vehicle | N/A | YES |
| 2 | Colchicine | 1 | YES |
| 3 | Indomethacin | 5 | YES |
| 4 | tranilast | 200 | YES |
| 5 | tranilast | 400 | YES |
| 6 | Vehicle | N/A | NO |

Thirty minutes after test material administration, the rats were anesthetized and five groups were injected into the air pouch with 15 ml MSU using an 18 G×1 inch needle fitted to a 20 ml syringe. The sixth group was injected with 15 ml saline. The rats were returned to their cages, no adverse affects were observed.

Four hours after MSU injection, the rats were anesthetized and 5 ml 10 U/ml heparinized saline was injected into the air pouch. The air pouch was gently massaged, the contents immediately removed using a 14 G×1 inch needle fitted to a 6 ml syringe, and the exudate volume recorded. An aliquot of the exudate was transferred to heparin-treated microtainer tubes (Becton Dickinson, Cat. 365958, lot 8093666, exp. June 09) for white blood cell (WBC) counting. The remainder of the exudate was centrifuged (Hermle Labrotechnik, Model Z200A, SN: 44060036), the supernatants removed and evaluated at OD₆₂₀ₙₘ (Spectronic Unicam, Model 4000 1/4, SN: 3SGD003006) for plasma extravasation.

TABLE 11
Average Exudate Volume (ml), WBC (cells/ml) and
Total WBC in Sample

| Group | Statistic | Exudate | WBC | Total WBC |
|---|---|---|---|---|
| 1 | Mean | 7.9 | 44150000 | 293500000 |
| | SD | 2.2 | 52211243 | 241424849 |
| 2 | Mean | 3.9 | 9215000 | 35131400 |
| | SD | 1.3 | 5110926 | 20711227 |
| 3 | Mean | 4.9 | 4160000 | 18520000 |
| | SD | 2.1 | 5556818 | 22309978 |
| 4 | Mean | 6.5 | 2524444 | 15925556 |
| | SD | 2.5 | 2236706 | 14881397 |
| 5 | Mean | 5.3 | 3144444 | 15162222 |
| | SD | 2.2 | 1473186 | 8878921 |
| 6 | Mean | 4.3 | 457000 | 1961600 |
| | SD | 0.2 | 299780 | 1242983 |

TABLE 12
Average Plasma Extravasation

| Group | Statistic | OD_{620 nm} |
|---|---|---|
| 1 | Mean | 1.612 |
| | SD | 0.309 |
| 2 | Mean | 0.776 |
| | SD | 0.239 |
| 3 | Mean | 0.505 |
| | SD | 0.203 |
| 4 | Mean | 0.697 |
| | SD | 0.324 |
| 5 | Mean | 0.626 |
| | SD | 0.221 |
| 6 | Mean | 0.004 |
| | SD | 0.015 |

### CONCLUSIONS

### Effect of MSU Injection into Six-Day Air Pouch

Four hours after MSU was injected into the six-day rat air pouch, 4.4×10⁷ WBC/ml were collected in 7.9 ml of exudate compared to 4.5×10⁵ WBC/ml in 4.3 ml exudate collected from animals which received an injection of saline instead of MSU. This was equivalent to a total WBC of 2.9×10⁸ in the MSU-treated rats relative to a total WBC of 2×10⁶ WBC in the saline control animals; approximately 150-fold increase in cell infiltration. The exudate collected from the MSU treated animals had an average OD₆₂₀ₙₘ of 1.612 relative to 0.004 OD in the saline treated rats, indicative of plasma extravasation associated with the inflammatory cell infiltration of the air pouch in response to MSU challenge.

Effect of Pretreatment with Colchicine:

Thirty minute pretreatment with 1 mg/kg colchicine, administered subcutaneously prior to MSU challenge, resulted in an 88% reduction in inflammatory cell infiltration associated with a 52% inhibition of plasma extravasation.

Effect of Pretreatment with Indomethacin:

Subcutaneous injection with 5 mg/kg indomethacin, thirty minutes prior to MSU challenge, yielded a 69% reduction of plasma extravasation accompanied by a 94% reduction in inflammatory cell infiltration into the air pouch.

Effect of Pretreatment with Tranilast:

Oral pretreatment with tranilast, thirty minutes prior to injection of MSU into the air pouch, resulted in a 60% inhibition of plasma extravasation and 95% inhibition of white blood cell infiltration of the gouty air pouch, regardless of dose.

In sum, pretreatment with orally administered tranilast, regardless of dose, was as effective as colchicine in reducing the plasma extravasation and as effective as indomethacin in preventing inflammatory cell infiltration of the air pouch in response to MSU challenge.

### Example 5

### Air Pouch Model 2

The objective of this study was to evaluate the anti-inflammatory potency of Tranilast. This evaluation was carried out in male Sprague-Dawley rats in a rodent model of gout. The animals were injected subcutaneously with 20 ml of sterile air, followed three days later by a supplemental injection with 20 ml of sterile air. Six days after the initial sterile air injection, the rats were injected intravenously with Evan's Blue and pretreated for thirty minutes with oral administration of 25 mg/kg, 50 mg/kg, 100 mg/kg or 200 mg/kg tranilast. After the pretreatment period, the rats were injected with 150 mg of monosodium urate (MSU) crystals (10 mg/ml) into the air pouch. Four hours later, the air pouch was injected with 5 ml heparinized saline and the entire contents of the air pouch removed, recording the total volume. The air pouch contents were evaluated for plasma extravasation and total white blood cell (WBC) count.

On day 0 the rats were anesthetized in a biological cabinet, the nape of the neck was cleansed with 70% isopropanol followed by iodine tincture (VEDCO, lot L021976). Twenty ml sterile (0.22 μm, Millipore, Cat. SLGSV255F, lot R8J1494130, exp July 2011) air was injected subcutaneously using a 23 G×2 inch needle fixed to a 20 ml syringe. The rats were returned to routine housing. No adverse reactions were observed.

On day 3 the rats were anesthetized in a biological cabinet, the nape of the neck was cleansed with 70% isopropanol followed by iodine tincture (VEDCO, lot L021976). Twenty ml sterile (0.22 μm, Fisher Scientific, Cat. 09-720-3, lot R5SN25683) air was injected subcutaneously using a 23 G×2 inch needle fixed to a 20 ml syringe. The rats were returned to routine housing. No adverse reactions were observed.

On day 6 the rats were weighed (Mettler, Model PE3000, SN: F69474) and injected intravenously injected with 2 ml/kg Evans Blue. The animals were dosed orally with 0.5 ml/kg, 1 ml/kg, 2; ml/kg or 4 ml/kg tranilast, or 4 ml/kg vehicle.

TABLE 13
Treatment Groups

| | | Dose | |
|---|---|---|---|
| Group | Treatment | (mg/kg) | +MSU |
| 1 | Vehicle | N/A | NO |
| 2 | Vehicle | N/A | YES |
| 3 | tranilast | 25 | YES |
| 4 | tranilast | 50 | YES |
| 5 | tranilast | 100 | YES |
| 6 | tranilast | 200 | YES |

Thirty minutes after test material administration, the rats were anesthetized and five groups (2-6) were injected into the air pouch with 15 ml MSU using a 19 G×2 inch needle fitted to a 20 ml syringe. Group 1 was injected with 15 ml saline. The rats were returned to their cages, no adverse affects were observed.

Four hours after MSU/saline injection, the rats were anesthetized and 5 ml 10 U/ml heparinized saline was injected into the air pouch. The air pouch was gently massaged, the contents immediately removed using a 14 G×1 inch needle fitted to a 6 ml syringe, and the exudate volume recorded. An aliquot of the exudate was transferred to heparin-treated microtainer tubes (Becton Dickinson, Cat. 365958, lot 9140110, expo July 2010) for white blood cell (WBC) counting. The remainder of the exudate was centrifuged (Hermle Labrotechnik, Model Z200A, SN: 44060036), the supernatants removed and evaluated at OD₆₂₀ₙₘ (Spectronic Unicam, Model 4000 1/4, SN: 3SGD003006) for plasma extravasation.

TABLE 14
Average Exudate Volume (ml), WBC (cells/ml) and
Total WBC in Sample

| Group | Statistic | Exudate | WBC | Total WBC |
|---|---|---|---|---|
| 1 | Mean | 4.0 | 218000 | 867630 |
| | SD | 0.2 | 164303 | 643063 |
| 2 | Mean | 8.4 | 6343000 | 51644700 |
| | SD | 1.8 | 5071566 | 44960412 |
| 3 | Mean | 6.5 | 3196000 | 20550000 |
| | SD | 1.6 | 2751497 | 21688826 |
| 4 | Mean | 8.6 | 1557000 | 12476000 |
| | SD | 2.0 | 1773553 | 14460749 |
| 5 | Mean | 8.0 | 1566000 | 10629600 |
| | SD | 1.8 | 1538449 | 9417750 |
| 6 | Mean | 3.2 | 3516000 | 10305200 |
| | SD | 0.7 | 3842442 | 10637627 |

TABLE 15
Average Plasma Extravasation

| Group | Statistic | OD_{620 nm} |
|---|---|---|
| 1 | Mean | −0002 |
| | SD | 0.015 |
| 2 | Mean | 1.420 |
| | SD | 0.499 |
| 3 | Mean | 1.431 |
| | SD | 0.271 |
| 4 | Mean | 1.048 |
| | SD | 0.224 |
| 5 | Mean | 0.939 |
| | SD | 0.300 |
| 6 | Mean | 0.819 |
| | SD | 0.277 |

Effect of MSU Injection into Six-Day Air Pouch:

Four hours after MSU was injected into the six-day rat air pouch, 6.3×10⁶ WBC/ml were collected in 8.4 ml of exudate compared to 2.2×10⁵ WBC/ml in 4 ml exudate collected from animals which received an injection of saline instead of MSU. This was equivalent to a total WBC of 5.2×10⁷ in the MSU-treated rats relative to a total WBC of 8.7×10⁵ WBC in the saline control animals; approximately 60-fold increase in cell infiltration. The exudate collected from the MSU treated animals had an average OD₆₂₀ₙₘ of 1.420 relative to −0.002 OD in the saline treated rats, indicative of plasma extravasation associated with the inflammatory cell infiltration of the air pouch in response to MSU challenge.

Effect of Pretreatment with Tranilast:

Oral pretreatment with tranilast, thirty minutes prior to injection of MSU into the air pouch, resulted in a dose-dependent inhibition of the MSU-induced parameters. At the highest dose (200 mg/kg) a 42% inhibition of plasma extravasation and 80% inhibition of white blood cell infiltration of the gouty air pouch was recorded. The data shows a dose-response trend of decreasing extravasation and decreasing white blood cell infiltration with increasing dose of tranilast as indicated by a significant result (P=0.0013 and P=0.0001, respectively) when analyzed by an Analysis of Variance Test (ANOVA). A Sigmoidal dose-response (variable slope) analysis of the data provides and IC₅₀ effect of tranilast on plasma extravasation if 45 mg/kg and on white blood cell infiltration of 16 mg/kg.

In sum, pretreatment with orally administered tranilast, resulted in a dose-dependent inhibition of plasma extravasation and leukocyte infiltration of the air pouch in response to MSU challenge.

### Example 6

### Air Pouch Model 3

The objective of this study was to evaluate the anti-inflammatory potency of Tranilast. This evaluation was carried out in male Sprague-Dawley rats in a rodent model of gout. The animals were injected subcutaneously with 20 ml of sterile air, followed three days later by a supplemental injection with 20 ml of sterile air. Six days after the initial sterile air injection, the rats were injected intravenously with Evan's Blue and pretreated for thirty minutes with oral administration of 25 mg/kg, 50 mg/kg, 100 mg/kg, 200 mg/kg, or 300 mg/kg tranilast. After the pretreatment period, the rats were injected with 150 mg of monosodium urate (MSU) crystals (10 mg/ml) into the air pouch. Four hours later, the air pouch was injected with 5 ml heparinized saline and the entire contents of the air pouch removed, recording the total volume. The air pouch contents were evaluated for plasma extravasation and total white blood cell (WBC) count.

On day 0 the rats were anesthetized in a biological cabinet, the nape of the neck was cleansed with 70% isopropanol followed by iodine tincture (VEDCO). Twenty ml sterile (0.22 μm, Millipore, Cat. SLGSV255F) air was injected subcutaneously using a 23 G×2 inch needle fixed to a 20 ml syringe. The rats were returned to routine housing. No adverse reactions were observed.

On day 3 the rats were anesthetized in a biological cabinet, the nape of the neck was cleansed with 70% isopropanol followed by iodine tincture (VEDCO). Twenty ml sterile (0.22 μm, Fisher Scientific, Cat. 09-720-3) air was injected subcutaneously using a 23 G×2 inch needle fixed to a 20 ml syringe. The rats were returned to routine housing. No adverse reactions were observed.

On day 6 the rats were weighed (Mettler, Model PE3000, SN: F69474) and injected intravenously injected with 2 ml/kg Evans Blue. The animals were dosed orally with 0.5 ml/kg, 1 ml/kg, 2 ml/kg, 4 ml/kg tranilast, or 6 ml/kg tranilast or 6 ml/kg vehicle.

TABLE 16
Treatment Groups

| | | Dose | |
|---|---|---|---|
| Group | Treatment | (mg/kg) | +MSU |
| 1 | Vehicle | N/A | NO |
| 2 | Vehicle | N/A | YES |
| 3 | tranilast | 25 | YES |
| 4 | tranilast | 50 | YES |
| 5 | tranilast | 100 | YES |
| 6 | tranilast | 200 | YES |
| 7 | tranilast | 300 | YES |

Thirty minutes after test material administration, the rats were anesthetized and six groups (2-7) were injected into the air pouch with 15 ml MSU using a 19 G×2 inch needle fitted to a 20 ml syringe. Group 1 was injected with 15 ml saline. The rats were returned to their cages, no adverse affects were observed.

Four hours after MSU/saline injection, the rats were anesthetized and 5 ml 10 U/ml heparinized saline was injected into the air pouch. The air pouch was gently massaged, the contents immediately removed using a 14 G×1 inch needle fitted to a 6 ml syringe, and the exudate volume recorded. An aliquot of the exudate was transferred to heparin-treated microtainer tubes (Becton Dickinson, Cat. 365958) for white blood cell (WBC) counting. The remainder of the exudate was centrifuged (Hermle Labrotechnik, Model Z200A, SN: 44060036), the supernatants removed and evaluated at OD₆₂₀ₙₘ (Spectronic Unicam, Model 4000 1/4, SN: 3SGD003006) for plasma extravasation.

TABLE 17
Average Exudate Volume (ml), WBC (cells/ml) and
Total WBC in Sample

| Group | Statistic | Exudate | WBC | Total WBC |
|---|---|---|---|---|
| 1 | Mean | 3.7 | 948000 | 3040400 |
| | SD | 0.6 | 2223690 | 6666456 |
| 2 | Mean | 8.4 | 36690000 | 302629000 |
| | SD | 1.8 | 32747466 | 284553989 |
| 3 | Mean | 6.9 | 31260000 | 204170000 |
| | SD | 1.9 | 26599549 | 174196900 |
| 4 | Mean | 4.8 | 18900000 | 96948000 |
| | SD | 1.4 | 12165616 | 77939099 |
| 5 | Mean | 6.4 | 27440000 | 145408000 |
| | SD | 3.0 | 28328988 | 129019202 |
| 6 | Mean | 6.2 | 12000000 | 71692000 |
| | SD | 1.8 | 11060641 | 68403802 |
| 7 | Mean | 5.6 | 5880000 | 29232000 |
| | SD | 2.6 | 5484686 | 24104106 |

TABLE 18
Average Plasma Extravasation

| Group | Statistic | OD_{620 nm} |
|---|---|---|
| 1 | Mean | 0.008 |
| | SD | 0.027 |
| 2 | Mean | 1.753 |
| | SD | 0.270 |
| 3 | Mean | 1.181 |
| | SD | 0.435 |
| 4 | Mean | 1.071 |
| | SD | 0.383 |
| 5 | Mean | 1.276 |
| | SD | 0.525 |
| 6 | Mean | 1.033 |
| | SD | 0.184 |
| 7 | Mean | 0.611 |
| | SD | 0.368 |

Effect of MSU Injection into Six-Day Air Pouch:

Four hours after MSU was injected into the six-day rat air pouch, 3.7×10⁷ WBC/ml were collected in 8.4 ml of exudate compared to 9.5×10⁵ WBC/ml in 3.7 ml exudate collected from animals which received an injection of saline instead of MSU. This was equivalent to a total WBC of 3.0×10⁸ in the MSU-treated rats relative to a total WBC of 3.0×10⁶ WBC in the saline control animals; a 100-fold increase in cell infiltration. The exudate collected from the MSU treated animals had an average OD₆₂₀ₙₘ of 1.753 relative to 0.008 OD in the saline treated rats, indicative of plasma extravasation associated with the inflammatory cell infiltration of the air pouch in response to MSU challenge.

Effect of Pretreatment with Tranilast:

Oral pretreatment with tranilast, thirty minutes prior to injection of MSU into the air pouch, resulted in a dose-dependent inhibition of the MSU-induced parameters. At the highest dose (300 mg/kg) a 65% inhibition of plasma extravasation and 90% inhibition of white blood cell infiltration of the gouty air pouch was recorded. In sum, pretreatment with orally administered tranilast, resulted in a dose-dependent inhibition of plasma extravasation and leukocyte infiltration of the air pouch in response to MSU challenge.

### Example 7

### Air Pouch Model 4

The objective of this study was to evaluate the anti-inflammatory potency of Tranilast. This evaluation was carried out in male Sprague-Dawley rats in a rodent model of gout. The animals were injected subcutaneously with 20 ml of sterile air, followed three days later by a supplemental injection with 20 ml of sterile air. Six days after the initial sterile air injection, the rats were injected intravenously with Evan's Blue and pretreated for thirty minutes with oral administration of 3 mg/kg, 10 mg/kg, 30 mg/kg, 100 mg/kg, or 300 mg/kg tranilast. After the pretreatment period, the rats were injected with 150 mg of monosodium urate (MSU) crystals (10 mg/ml) into the air pouch. Four hours later, the air pouch was injected with 5 ml heparinized saline and the entire contents of the air pouch removed, recording the total volume. The air pouch contents were evaluated for plasma extravasation and total white blood cell (WBC) count.

On day 0 the rats were anesthetized in a biological cabinet, the nape of the neck was cleansed with 70% isopropanol followed by iodine tincture (VEDCO). Twenty ml sterile (0.22 μm, Millipore, Cat. SLGSV255F) air was injected subcutaneously using a 23 G×2 inch needle fixed to a 20 ml syringe. The rats were returned to routine housing. No adverse reactions were observed.

On day 3 the rats were anesthetized in a biological cabinet, the nape of the neck was cleansed with 70% isopropanol followed by iodine tincture (VEDCO). Twenty ml sterile (0.22 μm, Fisher Scientific, Cat. 09-720-3) air was injected subcutaneously using a 23 G×2 inch needle fixed to a 20 ml syringe. The rats were returned to routine housing. No adverse reactions were observed.

On day 6 the rats were weighed (Mettler, Model PE3000, SN: F69474) and injected intravenously injected with 2 ml/kg Evans Blue. The animals were dosed orally with 0.06 ml/kg, 0.2 ml/kg, 0.6 ml/kg, 2 ml/kg, or 6 ml/kg tranilast or 6 ml/kg vehicle.

TABLE 19
Treatment Groups

| | | Dose | |
|---|---|---|---|
| Group | Treatment | (mg/kg) | +MSU |
| 1 | Vehicle | N/A | NO |
| 2 | Vehicle | N/A | YES |
| 3 | tranilast | 3 | YES |
| 4 | tranilast | 10 | YES |
| 5 | tranilast | 30 | YES |
| 6 | tranilast | 100 | YES |
| 7 | tranilast | 300 | YES |

Thirty minutes after test material administration, the rats were anesthetized and six groups (2-7) were injected into the air pouch with 15 ml MSU using a 19 G×2 inch needle fitted to a 20 ml syringe. Group 1 was injected with 15 ml saline. The rats were returned to their cages, no adverse affects were observed.

Four hours after MSU/saline injection, the rats were anesthetized and 5 ml 10 U/ml heparinized saline was injected into the air pouch. The air pouch was gently massaged, the contents immediately removed using a 14 G×1 inch needle fitted to a 6 ml syringe, and the exudate volume recorded. An aliquot of the exudate was transferred to heparin-treated microtainer tubes (Becton Dickinson, Cat. 365958) for white blood cell (WBC) counting. The remainder of the exudate was centrifuged (Hermle Labrotechnik, Model Z200A, SN: 44060036), the supernatants removed and evaluated at OD₆₂₀ₙₘ (Spectronic Unicam, Model 4000 1/4, SN: 3SGD003006) for plasma extravasation.

TABLE 20
Average Exudate Volume (ml), WBC (cells/ml) and
Total WBC in Sample

| | | | | Total |
|---|---|---|---|---|
| Group | Statistic | Exudate | WBC | WBC |
| 1 | Mean | 4.5 | 431000 | 1969900 |
| | SD | 0.6 | 381356 | 1795332 |
| 2 | Mean | 9.8 | 43360000 | 389336000 |
| | SD | 2.0 | 35267618 | 297225782 |
| 3 | Mean | 9.6 | 38510000 | 368228000 |
| | SD | 0.9 | 33458513 | 310223260 |
| 4 | Mean | 7.7 | 61200000 | 471436000 |
| | SD | 0.7 | 49824314 | 390429887 |
| 5 | Mean | 7.1 | 39650000 | 286935000 |
| | SD | 1.0 | 38622165 | 281432729 |
| 6 | Mean | 5.1 | 31740000 | 159916000 |
| | SD | 1.2 | 31730259 | 137413471 |
| 7 | Mean | 3.9 | 19466667 | 77451111 |
| | SD | 0.3 | 10047388 | 42189878 |

TABLE 21
Average Plasma Extravasation

| Group | Statistic | OD_{620 nm} |
|---|---|---|
| 1 | Mean | 0.028 |
| | SD | 0.007 |
| 2 | Mean | 1.845 |
| | SD | 0.468 |
| 3 | Mean | 1.940 |
| | SD | 0.365 |
| 4 | Mean | 1.662 |
| | SD | 0.498 |
| 5 | Mean | 1.089 |
| | SD | 0.418 |
| 6 | Mean | 1.180 |
| | SD | 0.483 |
| 7 | Mean | 0.613 |
| | SD | 0.314 |

Effect of MSU Injection into Six-Day Air Pouch:

Four hours after MSU was injected into the six-day rat air pouch, 4.3×10⁷ WBC/ml were collected in 9.8 ml of exudate compared to 4.3×10⁵ WBC/ml in 4.5 ml exudate collected from animals which received an injection of saline instead of MSU. This was equivalent to a total WBC of 3.9×10⁸ in the MSU-treated rats relative to a total WBC of 2×10⁶ WBC in the saline control animals; approximately 200-fold increase in cell infiltration. The exudate collected from the MSU treated animals had an average OD₆₂₀ₙₘ of 1.854 relative to 0.028 OD in the saline treated rats, indicative of plasma extravasation associated with the inflammatory cell infiltration of the air pouch in response to MSU challenge.

Effect of Pretreatment with Tranilast:

Oral pretreatment with tranilast, thirty minutes prior to injection of MSU into the air pouch, resulted in a dose-dependent inhibition of the MSU-induced parameters. At the highest dose (300 mg/kg) a 67% inhibition of plasma extravasation and 80% inhibition of white blood cell infiltration of the gouty air pouch was recorded. In sum, pretreatment with orally administered tranilast, resulted in a dose-dependent inhibition of plasma extravasation and leukocyte infiltration of the air pouch in response to MSU challenge.

While preferred embodiments of the present invention have been shown and described herein, it will be obvious to those skilled in the art that such embodiments are provided by way of example only.

Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A pharmaceutical composition comprising:
(a) a first therapeutic agent, wherein said first therapeutic agent is a compound of formula II: 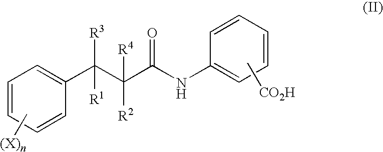
or a pharmaceutically acceptable salt thereof,
wherein each of R¹ and R² is independently selected from a hydrogen atom or a C₁-C₄ alkyl group, R³ and R⁴ are each hydrogen atoms or together form another chemical bond, each X is independently selected from a hydroxyl group, a halogen atom, a C₁-C₄ alkyl group or a C₁-C₄ alkoxy group, or when two X groups are alkyl or alkoxy groups, they may be connected together to form a ring, and n is an integer from 1 to 3;
(b) a second therapeutic agent, wherein said second therapeutic agent is a uric acid synthesis inhibitor or a uricosuric agent; and
(c) a pharmaceutically acceptable diluent or carrier.

2. A pharmaceutical composition according to claim 1, wherein said compound of formula II is selected from the group consisting of 2-[[3-(2-methylphenyl)-1-oxo-2-propenyl]amino]benzoic acid; 2-[[3-(3-methylphenyl)-1-oxo-2-propenyl]amino]benzoic acid; 2-[[3-(4-methylphenyl)-1-oxo-2-propenyl]amino]benzoic acid; 2-[[3-(2-ethylphenyl)-1-oxo-2-propenyl]amino]benzoic acid; 2-[[3-(3-ethylphenyl)-1-oxo-2-propenyl]amino]benzoic acid; 2-[[3-(4-ethylphenyl)-1-oxo-2-propenyl]amino]benzoic acid; 2-[[3-(2-propylphenyl)-1-oxo-2-propenyl]amino]benzoic acid; 2-[[3-(3-propylphenyl)-1-oxo-2-propenyl]amino]benzoic acid; 2-[[3-(4-propylphenyl)-1-oxo-2-propenyl]amino]benzoic acid; 2-[[3-(2-hydroxyphenyl)-1-oxo-2-propenyl]amino]benzoic acid; 2-[[3-(3-hydroxyphenyl)-1-oxo-2-propenyl]amino]benzoic acid; 2-[[3-(4-hydroxyphenyl)-1-oxo-2-propenyl]amino]benzoic acid; 2-[[3-(2-chlorophenyl)-1-oxo-2-propenyl]amino]benzoic acid; 2-[[3-(3-chlorophenyl)-1-oxo-2-propenyl]amino]benzoic acid; 2-[[3-(4-chlorophenyl)-1-oxo-2-propenyl]amino]benzoic acid; 2-[[3-(2-fluorophenyl)-1-oxo-2-propenyl]amino]benzoic acid; 2-[[3-(3-fluorophenyl)-1-oxo-2-propenyl]amino]benzoic acid; 2-[[3-(4-fluorophenyl)-1-oxo-2-propenyl]amino]benzoic acid; 2-[[3-(2-bromophenyl)-1-oxo-2-propenyl]amino]benzoic acid; 2-[[3-(3-bromophenyl)-1-oxo-2-propenyl]amino]benzoic acid; 2-[[3-(4-bromophenyl)-1-oxo-2-propenyl]amino]benzoic acid; 2-[[3-(2,3-dimethoxyphenyl)-1-oxo-2-propenyl]amino]benzoic acid; 2-[[3-(3,4-dimethoxyphenyl)-1-oxo-2-propenyl]amino]benzoic acid; 2-[[3-(2,4-dimethoxyphenyl)-1-oxo-2-propenyl]amino]benzoic acid; 2-[[3-(2,3-dimethylphenyl)-1-oxo-2-propenyl]amino]benzoic acid; 2-[[3-(3,4-dimethylphenyl)-1-oxo-2-propenyl]amino]benzoic acid; 2-[[3-(2,4-dimethylphenyl)-1-oxo-2-propenyl]amino]benzoic acid; 2-[[3-(2,3-diethoxyphenyl)-1-oxo-2-propenyl]amino]benzoic acid; 2-[[3-(3,4-diethoxyphenyl)-1-oxo-2-propenyl]amino]benzoic acid; 2-[[3-(2,4-diethoxyphenyl)-1-oxo-2-propenyl]amino]benzoic acid; 2-[[3-(2,3-dipropoxyphenyl)-1-oxo-2-propenyl]amino]benzoic acid; 2-[[3-(3,4-dipropoxyphenyl)-1-oxo-2-propenyl]amino]benzoic acid; 2-[[3-(2,4-dipropoxyphenyl)-1-oxo-2-propenyl]amino]benzoic acid; 2-[[3-(2,3-diethylphenyl)-1-oxo-2-propenyl]amino]benzoic acid; 2-[[3-(3,4-diethylphenyl)-1-oxo-2-propenyl]amino]benzoic acid; 2-[[3-(2,4-diethylphenyl)-1-oxo-2-propenyl]amino]benzoic acid; 2-[[3-(2,3-dipropylphenyl)-1-oxo-2-propenyl]amino]benzoic acid; 2-[[3-(3,4-dipropylphenyl)-1-oxo-2-propenyl]amino]benzoic acid; 2-[[3-(2,4-dipropylphenyl)-1-oxo-2-propenyl]amino]benzoic acid; 2-[[3-(2-methoxy-3-methylphenyl)-1-oxo-2-propenyl]amino]benzoic acid, 2-[[3-(3-methoxy-4-methylphenyl)-1-oxo-2-propenyl]amino]benzoic acid; 2-[[3-(2-methoxy-3-methylphenyl)-1-oxo-2-propenyl]amino]benzoic acid; 2-[[3-(2-methoxy-4-methylphenyl)-1-oxo-2-propenyl]amino]benzoic acid; 2-[[3-(2-methoxy-3-chlorophenyl)-1-oxo-2-propenyl]amino]benzoic acid; 2-[[3-(3-methoxy-4-chlorophenyl)-1-oxo-2-propenyl]amino]benzoic acid; 2-[[3-(2-methoxy-3-chlorophenyl)-1-oxo-2-propenyl]amino]benzoic acid; 2-[[3-(2-methoxy-4-chlorophenyl)-1-oxo-2-propenyl]amino]benzoic acid; 2-[[3-(2-methoxy-3-hydroxyphenyl)-1-oxo-2-propenyl]amino]benzoic acid; 2-[[3-(3-methoxy-4-hydroxyphenyl)-1-oxo-2-propenyl]amino]benzoic acid; 2-[[3-(2-methoxy-3-hydroxyphenyl)-1-oxo-2-propenyl]amino]benzoic acid; 2-[[3-(2-methoxy-4-hydroxyphenyl)-1-oxo-2-propenyl]amino]benzoic acid; 2-[[3-(3,4-trimethylenephenyl)-1-oxo-2-propenyl]amino]benzoic acid; 2-[[3-(2,3-trimethylenephenyl)-1-oxo-2-propenyl]amino]benzoic acid; 2-[[3-(3,4-methylenedioxyphenyl)-1-oxo-2-propenyl]amino]benzoic acid; and 2-[[3-(3,4-ethylenedioxyphenyl)-1-oxo-2-propenyl]amino]benzoic acid.

3. A pharmaceutical composition according to claim 2, wherein said compound of formula II is 2-[[3-(3,4-dimethoxyphenyl)-1-oxo-2-propenyl]amino]benzoic acid (tranilast).

4. A pharmaceutical composition according to claim 3, wherein said second therapeutic agent is a uric acid synthesis inhibitor.

5. A pharmaceutical composition according to claim 4, wherein said uric acid synthesis inhibitor is a xanthine oxidase inhibitor.

6. A pharmaceutical composition according to claim 5, wherein said xanthine oxidase inhibitor is allopurinol, febuxostat, oxypurinol, tisopurine or an inositol.

7. A pharmaceutical composition according to claim 6, wherein said xanthine oxidase inhibitor is allopurinol or febuxostat.

8. A pharmaceutical composition according to claim 7, wherein said xanthine oxidase inhibitor is allopurinol.

9. A pharmaceutical composition according to claim 8, wherein said composition comprises from about 100 mg to about 300 mg of tranilast.

10. A pharmaceutical composition according to claim 9, wherein said composition comprises from about 100 mg to about 300 mg of allopurinol.

11. A pharmaceutical composition according to claim 3, wherein said second therapeutic agent is a uricosuric agent.

12. A pharmaceutical composition according to claim 11, wherein said uricosuric agent is probenecid, benzbromarone, sulfinpyrazone, guaifenesin, losartan, atorvastatin, amlodipine, adrenocorticotropic hormone or fenofibrate.

13. A pharmaceutical composition according to claim 12, wherein said uricosuric agent is probenecid.

14. A method of treating a condition associated with an elevated serum uric acid level comprising administering to a subject in need thereof a pharmaceutical composition comprising:
(a) a first therapeutic agent, wherein said first therapeutic agent is a compound of formula II: 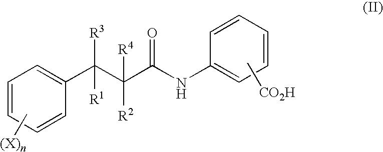
or a pharmaceutically acceptable salt thereof,
wherein each of R¹ and R² is independently selected from a hydrogen atom or a C₁-C₄ alkyl group, R³ and R⁴ are each hydrogen atoms or together form another chemical bond, each X is independently selected from a hydroxyl group, a halogen atom, a C₁-C₄ alkyl group or a C₁-C₄ alkoxy group, or when two X groups are alkyl or alkoxy groups, they may be connected together to form a ring, and n is an integer from 1 to 3;
(b) a second therapeutic agent, wherein said second therapeutic agent is a uric acid synthesis inhibitor or a uricosuric agent; and
(c) a pharmaceutically acceptable diluent or carrier.

15. A method according to claim 14, wherein said first therapeutic agent is selected from the group consisting of 2-[[3-(2-methylphenyl)-1-oxo-2-propenyl]amino]benzoic acid; 2-[[3-(3-methylphenyl)-1-oxo-2-propenyl]amino]benzoic acid; 2-[[3-(4-methylphenyl)-1-oxo-2-propenyl]amino]benzoic acid; 2-[[3-(2-ethylphenyl)-1-oxo-2-propenyl]amino]benzoic acid; 2-[[3-(3-ethylphenyl)-1-oxo-2-propenyl]amino]benzoic acid; 2-[[3-(4-ethylphenyl)-1-oxo-2-propenyl]amino]benzoic acid; 2-[[3-(2-propylphenyl)-1-oxo-2-propenyl]amino]benzoic acid; 2-[[3-(3-propylphenyl)-1-oxo-2-propenyl]amino]benzoic acid; 2-[[3-(4-propylphenyl)-1-oxo-2-propenyl]amino]benzoic acid; 2-[[3-(2-hydroxyphenyl)-1-oxo-2-propenyl]amino]benzoic acid; 2-[[3-(3-hydroxyphenyl)-1-oxo-2-propenyl]amino]benzoic acid; 2-[[3-(4-hydroxyphenyl)-1-oxo-2-propenyl]amino]benzoic acid; 2-[[3-(2-chlorophenyl)-1-oxo-2-propenyl]amino]benzoic acid; 2-[[3-(3-chlorophenyl)-1-oxo-2-propenyl]amino]benzoic acid; 2-[[3-(4-chlorophenyl)-1-oxo-2-propenyl]amino]benzoic acid; 2-[[3-(2-fluorophenyl)-1-oxo-2-propenyl]amino]benzoic acid; 2-[[3-(3-fluorophenyl)-1-oxo-2-propenyl]amino]benzoic acid; 2-[[3-(4-fluorophenyl)-1-oxo-2-propenyl]amino]benzoic acid; 2-[[3-(2-bromophenyl)-1-oxo-2-propenyl]amino]benzoic acid; 2-[[3-(3-bromophenyl)-1-oxo-2-propenyl]amino]benzoic acid; 2-[[3-(4-bromophenyl)-1-oxo-2-propenyl]amino]benzoic acid; 2-[[3-(2,3-dimethoxyphenyl)-1-oxo-2-propenyl]amino]benzoic acid; 2-[[3-(3,4-dimethoxyphenyl)-1-oxo-2-propenyl]amino]benzoic acid; 2-[[3-(2,4-dimethoxyphenyl)-1-oxo-2-propenyl]amino]benzoic acid; 2-[[3-(2,3-dimethylphenyl)-1-oxo-2-propenyl]amino]benzoic acid; 2-[[3-(3,4-dimethylphenyl)-1-oxo-2-propenyl]amino]benzoic acid; 2-[[3-(2,4-dimethylphenyl)-1-oxo-2-propenyl]amino]benzoic acid; 2-[[3-(2,3-diethoxyphenyl)-1-oxo-2-propenyl]amino]benzoic acid; 2-[[3-(3,4-diethoxyphenyl)-1-oxo-2-propenyl]amino]benzoic acid; 2-[[3-(2,4-diethoxyphenyl)-1-oxo-2-propenyl]amino]benzoic acid; 2-[[3-(2,3-dipropoxyphenyl)-1-oxo-2-propenyl]amino]benzoic acid; 2-[[3-(3,4-dipropoxyphenyl)-1-oxo-2-propenyl]amino]benzoic acid; 2-[[3-(2,4-dipropoxyphenyl)-1-oxo-2-propenyl]amino]benzoic acid; 2-[[3-(2,3-diethylphenyl)-1-oxo-2-propenyl]amino]benzoic acid; 2-[[3-(3,4-diethylphenyl)-1-oxo-2-propenyl]amino]benzoic acid; 2-[[3-(2,4-diethylphenyl)-1-oxo-2-propenyl]amino]benzoic acid; 2-[[3-(2,3-dipropylphenyl)-1-oxo-2-propenyl]amino]benzoic acid; 2-[[3-(3,4-dipropylphenyl)-1-oxo-2-propenyl]amino]benzoic acid; 2-[[3-(2,4-dipropylphenyl)-1-oxo-2-propenyl]amino]benzoic acid; 2-[[3-(2-methoxy-3-methylphenyl)-1-oxo-2-propenyl]amino]benzoic acid, 2-[[3-(3-methoxy-4-methylphenyl)-1-oxo-2-propenyl]amino]benzoic acid; 2-[[3-(2-methoxy-3-methylphenyl)-1-oxo-2-propenyl]amino]benzoic acid; 2-[[3-(2-methoxy-4-methylphenyl)-1-oxo-2-propenyl]amino]benzoic acid; 2-[[3-(2-methoxy-3-chlorophenyl)-1-oxo-2-propenyl]amino]benzoic acid; 2-[[3-(3-methoxy-4-chlorophenyl)-1-oxo-2-propenyl]amino]benzoic acid; 2-[[3-(2-methoxy-3-chlorophenyl)-1-oxo-2-propenyl]amino]benzoic acid; 2-[[3-(2-methoxy-4-chlorophenyl)-1-oxo-2-propenyl]amino]benzoic acid; 2-[[3-(2-methoxy-3-hydroxyphenyl)-1-oxo-2-propenyl]amino]benzoic acid; 2-[[3-(3-methoxy-4-hydroxyphenyl)-1-oxo-2-propenyl]amino]benzoic acid; 2-[[3-(2-methoxy-3-hydroxyphenyl)-1-oxo-2-propenyl]amino]benzoic acid; 2-[[3-(2-methoxy-4-hydroxyphenyl)-1-oxo-2-propenyl]amino]benzoic acid; 2-[[3-(3,4-trimethylenephenyl)-1-oxo-2-propenyl]amino]benzoic acid; 2-[[3-(2,3-trimethylenephenyl)-1-oxo-2-propenyl]amino]benzoic acid; 2-[[3-(3,4-methylenedioxyphenyl)-1-oxo-2-propenyl]amino]benzoic acid; and 2-[[3-(3,4-ethylenedioxyphenyl)-1-oxo-2-propenyl]amino]benzoic acid.

16. A method according to claim 15, wherein said compound of formula II is 2-[[3-(3,4-dimethoxyphenyl)-1-oxo-2-propenyl]amino]benzoic acid (tranilast).

17. A method according to claim 16, wherein said second therapeutic agent is a uric acid synthesis inhibitor.

18. A method according to claim 17, wherein said uric acid synthesis inhibitor is a xanthine oxidase inhibitor.

19. A method according to claim 18, wherein said xanthine oxidase inhibitor is allopurinol, febuxostat, oxypurinol, tisopurine or an inositol.

20. A method according to claim 19, wherein said xanthine oxidase inhibitor is allopurinol or febuxostat.

21. A method according to claim 20, wherein said xanthine oxidase inhibitor is allopurinol.

22. A method according to claim 16, wherein said condition is hyperuricemia or gout.

23. A method according to claim 22, wherein said condition is hyperuricemia.

24. A method according to claim 22, wherein said condition is gout.

25. A method according to claim 24, wherein said gout is severe gout.

26. A method according to claim 24, wherein said gout is chronic gout.

27. A method according to claim 24, wherein said acute gout.

28. A method of treating a condition associated with an elevated serum uric acid level comprising administering to a subject in need thereof a pharmaceutically effective amount of a compound of formula II: 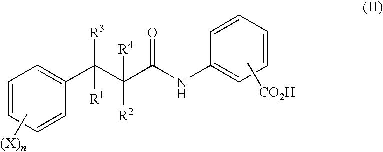
or a pharmaceutically acceptable salt thereof,
wherein each of R¹ and R² is independently selected from a hydrogen atom or a C₁-C₄ alkyl group, R³ and R⁴ are each hydrogen atoms or together form another chemical bond, each X is independently selected from a hydroxyl group, a halogen atom, a C₁-C₄ alkyl group or a C₁-C₄ alkoxy group, or when two X groups are alkyl or alkoxy groups, they may be connected together to form a ring, and n is an integer from 1 to 3.

29. A method according to claim 28, wherein said condition is hyperuricemia or gout.

30. A method according to claim 29, wherein said condition is hyperuricemia.

31. A method according to claim 29, wherein said condition is gout.

32. A method according to claim 31, wherein said gout is severe gout.

33. A method according to claim 31, wherein said gout is chronic gout.

34. A method according to claim 31, wherein said gout is acute gout.

35. A method according to claim 28, wherein said compound is selected from the group consisting of 2-[[3-(2-methylphenyl)-1-oxo-2-propenyl]amino]benzoic acid; 2-[[3-(3-methylphenyl)-1-oxo-2-propenyl]amino]benzoic acid; 2-[[3-(4-methylphenyl)-1-oxo-2-propenyl]amino]benzoic acid; 2-[[3-(2-ethylphenyl)-1-oxo-2-propenyl]amino]benzoic acid; 2-[[3-(3-ethylphenyl)-1-oxo-2-propenyl]amino]benzoic acid; 2-[[3-(4-ethylphenyl)-1-oxo-2-propenyl]amino]benzoic acid; 2-[[3-(2-propylphenyl)-1-oxo-2-propenyl]amino]benzoic acid; 2-[[3-(3-propylphenyl)-1-oxo-2-propenyl]amino]benzoic acid; 2-[[3-(4-propylphenyl)-1-oxo-2-propenyl]amino]benzoic acid; 2-[[3-(2-hydroxyphenyl)-1-oxo-2-propenyl]amino]benzoic acid; 2-[[3-(3-hydroxyphenyl)-1-oxo-2-propenyl]amino]benzoic acid; 2-[[3-(4-hydroxyphenyl)-1-oxo-2-propenyl]amino]benzoic acid; 2-[[3-(2-chlorophenyl)-1-oxo-2-propenyl]amino]benzoic acid; 2-[[3-(3-chlorophenyl)-1-oxo-2-propenyl]amino]benzoic acid; 2-[[3-(4-chlorophenyl)-1-oxo-2-propenyl]amino]benzoic acid; 2-[[3-(2-fluorophenyl)-1-oxo-2-propenyl]amino]benzoic acid; 2-[[3-(3-fluorophenyl)-1-oxo-2-propenyl]amino]benzoic acid; 2-[[3-(4-fluorophenyl)-1-oxo-2-propenyl]amino]benzoic acid; 2-[[3-(2-bromophenyl)-1-oxo-2-propenyl]amino]benzoic acid; 2-[[3-(3-bromophenyl)-1-oxo-2-propenyl]amino]benzoic acid; 2-[[3-(4-bromophenyl)-1-oxo-2-propenyl]amino]benzoic acid; 2-[[3-(2,3-dimethoxyphenyl)-1-oxo-2-propenyl]amino]benzoic acid; 2-[[3-(3,4-dimethoxyphenyl)-1-oxo-2-propenyl]amino]benzoic acid; 2-[[3-(2,4-dimethoxyphenyl)-1-oxo-2-propenyl]amino]benzoic acid; 2-[[3-(2,3-dimethylphenyl)-1-oxo-2-propenyl]amino]benzoic acid; 2-[[3-(3,4-dimethylphenyl)-1-oxo-2-propenyl]amino]benzoic acid; 2-[[3-(2,4-dimethylphenyl)-1-oxo-2-propenyl]amino]benzoic acid; 2-[[3-(2,3-diethoxyphenyl)-1-oxo-2-propenyl]amino]benzoic acid; 2-[[3-(3,4-diethoxyphenyl)-1-oxo-2-propenyl]amino]benzoic acid; 2-[[3-(2,4-diethoxyphenyl)-1-oxo-2-propenyl]amino]benzoic acid; 2-[[3-(2,3-dipropoxyphenyl)-1-oxo-2-propenyl]amino]benzoic acid; 2-[[3-(3,4-dipropoxyphenyl)-1-oxo-2-propenyl]amino]benzoic acid; 2-[[3-(2,4-dipropoxyphenyl)-1-oxo-2-propenyl]amino]benzoic acid; 2-[[3-(2,3-diethylphenyl)-1-oxo-2-propenyl]amino]benzoic acid; 2-[[3-(3,4-diethylphenyl)-1-oxo-2-propenyl]amino]benzoic acid; 2-[[3-(2,4-diethylphenyl)-1-oxo-2-propenyl]amino]benzoic acid; 2-[[3-(2,3-dipropylphenyl)-1-oxo-2-propenyl]amino]benzoic acid; 2-[[3-(3,4-dipropylphenyl)-1-oxo-2-propenyl]amino]benzoic acid; 2-[[3-(2,4-dipropylphenyl)-1-oxo-2-propenyl]amino]benzoic acid; 2-[[3-(2-methoxy-3-methylphenyl)-1-oxo-2-propenyl]amino]benzoic acid, 2-[[3-(3-methoxy-4-methylphenyl)-1-oxo-2-propenyl]amino]benzoic acid; 2-[[3-(2-methoxy-3-methylphenyl)-1-oxo-2-propenyl]amino]benzoic acid; 2-[[3-(2-methoxy-4-methylphenyl)-1-oxo-2-propenyl]amino]benzoic acid; 2-[[3-(2-methoxy-3-chlorophenyl)-1-oxo-2-propenyl]amino]benzoic acid; 2-[[3-(3-methoxy-4-chlorophenyl)-1-oxo-2-propenyl]amino]benzoic acid; 2-[[3-(2-methoxy-3-chlorophenyl)-1-oxo-2-propenyl]amino]benzoic acid; 2-[[3-(2-methoxy-4-chlorophenyl)-1-oxo-2-propenyl]amino]benzoic acid; 2-[[3-(2-methoxy-3-hydroxyphenyl)-1-oxo-2-propenyl]amino]benzoic acid; 2-[[3-(3-methoxy-4-hydroxyphenyl)-1-oxo-2-propenyl]amino]benzoic acid; 2-[[3-(2-methoxy-3-hydroxyphenyl)-1-oxo-2-propenyl]amino]benzoic acid; 2-[[3-(2-methoxy-4-hydroxyphenyl)-1-oxo-2-propenyl]amino]benzoic acid; 2-[[3-(3,4-trimethylenephenyl)-1-oxo-2-propenyl]amino]benzoic acid; 2-[[3-(2,3-trimethylenephenyl)-1-oxo-2-propenyl]amino]benzoic acid; 2-[[3-(3,4-methylenedioxyphenyl)-1-oxo-2-propenyl]amino]benzoic acid; and 2-[[3-(3,4-ethylenedioxyphenyl)-1-oxo-2-propenyl]amino]benzoic acid.

36. A method according to claim 35, wherein said compound is 2-[[3-(3,4-dimethoxyphenyl)-1-oxo-2-propenyl]amino]benzoic acid (tranilast).

37. A method according to claim 36, wherein said tranilast is administered in an amount from about 100 mg/day to about 900 mg/day.

38. A method according to claim 36, further comprising administering to said subject a second therapeutic agent, wherein said second therapeutic agent is a uric acid synthesis inhibitor or a uricosuric agent.

39. A method according to claim 38, wherein said second therapeutic agent is a uric acid synthesis inhibitor.

40. A method according to claim 39, wherein said uric acid synthesis inhibitor is a xanthine oxidase inhibitor.

41. A method according to claim 40, wherein said xanthine oxidase inhibitor is allopurinol, febuxostat, oxypurinol, tisopurine or an inositol.

42. A method according to claim 41, wherein said xanthine oxidase inhibitor is allopurinol.

43. A method according to claim 42, wherein said allopurinol is administered in an amount from about 100 mg/day to about 300 mg/day.

44. A method according to claim 41, wherein said xanthine oxidase inhibitor is febuxostat.

45. A method according to claim 44, wherein said febuxostat is administered in an amount from about 40 mg/day to about 80 mg/day.

46. A method according to claim 38, wherein said second therapeutic agent is a uricosuric agent.

47. A method according to claim 46, wherein said uricosuric agent is probenecid, benzbromarone, sulfinpyrazone, guaifenesin, losartan, atorvastatin, amlodipine, adrenocorticotropic hormone (ACTH) or fenofibrate.

48. A method according to claim 47, wherein said uricosuric agent is probenecid.

49. A method of decreasing serum uric acid level in a subject having a condition associated with an elevated serum uric acid level comprising administering to a subject in need thereof a pharmaceutical composition comprising:
(a) a first therapeutic agent, wherein said first therapeutic agent is a compound of formula II: 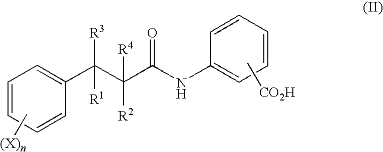
or a pharmaceutically acceptable salt thereof,
wherein each of R¹ and R² is independently selected from a hydrogen atom or a C₁-C₄ alkyl group, R³ and R⁴ are each hydrogen atoms or together form another chemical bond, each X is independently selected from a hydroxyl group, a halogen atom, a C₁-C₄ alkyl group or a C₁-C₄ alkoxy group, or when two X groups are alkyl or alkoxy groups, they may be connected together to form a ring, and n is an integer from 1 to 3;
(b) a second therapeutic agent, wherein said second therapeutic agent is a uric acid synthesis inhibitor or a uricosuric agent; and
(c) a pharmaceutically acceptable diluent or carrier.

50. A method according to claim 49, wherein said condition is hyperuricemia or gout.

51. A method according to claim 50, wherein said condition is hyperuricemia.

52. A method according to claim 50, wherein said condition is gout.

53. A method according to claim 52, wherein said gout is severe gout.

54. A method according to claim 52, wherein said gout is chronic gout.

55. A method according to claim 52, wherein said gout is acute gout.

56. A method of decreasing serum uric acid level in a subject having a condition associated with an elevated serum uric acid level comprising administering to a subject in need thereof a pharmaceutically effective amount of a compound of formula II: 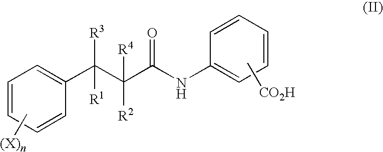
or a pharmaceutically acceptable salt thereof,
wherein each of R¹ and R² is independently selected from a hydrogen atom or a C₁-C₄ alkyl group, R³ and R⁴ are each hydrogen atoms or together form another chemical bond, each X is independently selected from a hydroxyl group, a halogen atom, a C₁-C₄ alkyl group or a C₁-C₄ alkoxy group, or when two X groups are alkyl or alkoxy groups, they may be connected together to form a ring, and n is an integer from 1 to 3.

57. A method according to claim 56, wherein said condition is hyperuricemia or gout.

58. A method according to claim 57, wherein said condition is hyperuricemia.

59. A method according to claim 57, wherein said condition is gout.

60. A method according to claim 59, wherein said gout is severe gout.

61. A method according to claim 59, wherein said gout is chronic gout.

62. A method according to claim 59, wherein said gout is acute gout.

63. A method according to claim 56, wherein said compound is selected from the group consisting of 2-[[3-(2-methylphenyl)-1-oxo-2-propenyl]amino]benzoic acid; 2-[[3-(3-methylphenyl)-1-oxo-2-propenyl]amino]benzoic acid; 2-[[3-(4-methylphenyl)-1-oxo-2-propenyl]amino]benzoic acid; 2-[[3-(2-ethylphenyl)-1-oxo-2-propenyl]amino]benzoic acid; 2-[[3-(3-ethylphenyl)-1-oxo-2-propenyl]amino]benzoic acid; 2-[[3-(4-ethylphenyl)-1-oxo-2-propenyl]amino]benzoic acid; 2-[[3-(2-propylphenyl)-1-oxo-2-propenyl]amino]benzoic acid; 2-[[3-(3-propylphenyl)-1-oxo-2-propenyl]amino]benzoic acid; 2-[[3-(4-propylphenyl)-1-oxo-2-propenyl]amino]benzoic acid; 2-[[3-(2-hydroxyphenyl)-1-oxo-2-propenyl]amino]benzoic acid; 2-[[3-(3-hydroxyphenyl)-1-oxo-2-propenyl]amino]benzoic acid; 2-[[3-(4-hydroxyphenyl)-1-oxo-2-propenyl]amino]benzoic acid; 2-[[3-(2-chlorophenyl)-1-oxo-2-propenyl]amino]benzoic acid; 2-[[3-(3-chlorophenyl)-1-oxo-2-propenyl]amino]benzoic acid; 2-[[3-(4-chlorophenyl)-1-oxo-2-propenyl]amino]benzoic acid; 2-[[3-(2-fluorophenyl)-1-oxo-2-propenyl]amino]benzoic acid; 2-[[3-(3-fluorophenyl)-1-oxo-2-propenyl]amino]benzoic acid; 2-[[3-(4-fluorophenyl)-1-oxo-2-propenyl]amino]benzoic acid; 2-[[3-(2-bromophenyl)-1-oxo-2-propenyl]amino]benzoic acid; 2-[[3-(3-bromophenyl)-1-oxo-2-propenyl]amino]benzoic acid; 2-[[3-(4-bromophenyl)-1-oxo-2-propenyl]amino]benzoic acid; 2-[[3-(2,3-dimethoxyphenyl)-1-oxo-2-propenyl]amino]benzoic acid; 2-[[3-(3,4-dimethoxyphenyl)-1-oxo-2-propenyl]amino]benzoic acid; 2-[[3-(2,4-dimethoxyphenyl)-1-oxo-2-propenyl]amino]benzoic acid; 2-[[3-(2,3-dimethylphenyl)-1-oxo-2-propenyl]amino]benzoic acid; 2-[[3-(3,4-dimethylphenyl)-1-oxo-2-propenyl]amino]benzoic acid; 2-[[3-(2,4-dimethylphenyl)-1-oxo-2-propenyl]amino]benzoic acid; 2-[[3-(2,3-diethoxyphenyl)-1-oxo-2-propenyl]amino]benzoic acid; 2-[[3-(3,4-diethoxyphenyl)-1-oxo-2-propenyl]amino]benzoic acid; 2-[[3-(2,4-diethoxyphenyl)-1-oxo-2-propenyl]amino]benzoic acid; 2-[[3-(2,3-dipropoxyphenyl)-1-oxo-2-propenyl]amino]benzoic acid; 2-[[3-(3,4-dipropoxyphenyl)-1-oxo-2-propenyl]amino]benzoic acid; 2-[[3-(2,4-dipropoxyphenyl)-1-oxo-2-propenyl]amino]benzoic acid; 2-[[3-(2,3-diethylphenyl)-1-oxo-2-propenyl]amino]benzoic acid; 2-[[3-(3,4-diethylphenyl)-1-oxo-2-propenyl]amino]benzoic acid; 2-[[3-(2,4-diethylphenyl)-1-oxo-2-propenyl]amino]benzoic acid; 2-[[3-(2,3-dipropylphenyl)-1-oxo-2-propenyl]amino]benzoic acid; 2-[[3-(3,4-dipropylphenyl)-1-oxo-2-propenyl]amino]benzoic acid; 2-[[3-(2,4-dipropylphenyl)-1-oxo-2-propenyl]amino]benzoic acid; 2-[[3-(2-methoxy-3-methylphenyl)-1-oxo-2-propenyl]amino]benzoic acid, 2-[[3-(3-methoxy-4-methylphenyl)-1-oxo-2-propenyl]amino]benzoic acid; 2-[[3-(2-methoxy-3-methylphenyl)-1-oxo-2-propenyl]amino]benzoic acid; 2-[[3-(2-methoxy-4-methylphenyl)-1-oxo-2-propenyl]amino]benzoic acid; 2-[[3-(2-methoxy-3-chlorophenyl)-1-oxo-2-propenyl]amino]benzoic acid; 2-[[3-(3-methoxy-4-chlorophenyl)-1-oxo-2-propenyl]amino]benzoic acid; 2-[[3-(2-methoxy-3-chlorophenyl)-1-oxo-2-propenyl]amino]benzoic acid; 2-[[3-(2-methoxy-4-chlorophenyl)-1-oxo-2-propenyl]amino]benzoic acid; 2-[[3-(2-methoxy-3-hydroxyphenyl)-1-oxo-2-propenyl]amino]benzoic acid; 2-[[3-(3-methoxy-4-hydroxyphenyl)-1-oxo-2-propenyl]amino]benzoic acid; 2-[[3-(2-methoxy-3-hydroxyphenyl)-1-oxo-2-propenyl]amino]benzoic acid; 2-[[3-(2-methoxy-4-hydroxyphenyl)-1-oxo-2-propenyl]amino]benzoic acid; 2-[[3-(3,4-trimethylenephenyl)-1-oxo-2-propenyl]amino]benzoic acid; 2-[[3-(2,3-trimethylenephenyl)-1-oxo-2-propenyl]amino]benzoic acid; 2-[[3-(3,4-methylenedioxyphenyl)-1-oxo-2-propenyl]amino]benzoic acid; and 2-[[3-(3,4-ethylenedioxyphenyl)-1-oxo-2-propenyl]amino]benzoic acid.

64. A method according to claim 63, wherein said compound is 2-[[3-(3,4-dimethoxyphenyl)-1-oxo-2-propenyl]amino]benzoic acid (tranilast).

65. A method according to claim 64, wherein said tranilast is administered in an amount from about 100 mg/day to about 900 mg/day.

66. A method according to claim 64, further comprising administering to said subject a second therapeutic agent, wherein said second therapeutic agent is a uric acid synthesis inhibitor or a uricosuric agent.

67. A method according to claim 66, wherein said second therapeutic agent is a uric acid synthesis inhibitor.

68. A method according to claim 67, wherein said uric acid synthesis inhibitor is a xanthine oxidase inhibitor.

69. A method according to claim 68, wherein said xanthine oxidase inhibitor is allopurinol, febuxostat, oxypurinol, tisopurine or an inositol.

70. A method according to claim 69, wherein said xanthine oxidase inhibitor is allopurinol.

71. A method according to claim 70, wherein said allopurinol is administered in an amount from about 100 mg/day to about 300 mg/day.

72. A method according to claim 69, wherein said xanthine oxidase inhibitor is febuxostat.

73. A method according to claim 72, wherein said febuxostat is administered in an amount from about 40 mg/day to about 80 mg/day.

74. A method according to claim 66, wherein said second therapeutic agent is a uricosuric agent.

75. A method according to claim 74, wherein said uricosuric agent is probenecid, benzbromarone, sulfinpyrazone, guaifenesin, losartan, atorvastatin, amlodipine, adrenocorticotropic hormone (ACTH) or fenofibrate.

76. A method according to claim 75, wherein said uricosuric agent is probenecid.

77. A method of decreasing serum uric acid level in a subject comprising administering to a subject in need thereof a pharmaceutically effective amount of a compound of formula II: 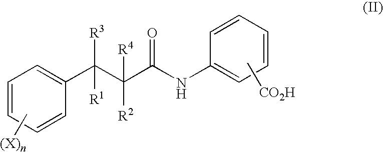
or a pharmaceutically acceptable salt thereof,
wherein each of R¹ and R² is independently selected from a hydrogen atom or a C₁-C₄ alkyl group, R³ and R⁴ are each hydrogen atoms or together form another chemical bond, each X is independently selected from a hydroxyl group, a halogen atom, a C₁-C₄ alkyl group or a C₁-C₄ alkoxy group, or when two X groups are alkyl or alkoxy groups, they may be connected together to form a ring, and n is an integer from 1 to 3.

78. A method according to claim 77, wherein said subject has hyperuricemia or gout.

79. A method according to claim 77, wherein said subject has a normal serum uric acid level.

80. A method according to claim 77, further comprising administering to said subject a second therapeutic agent, wherein said second therapeutic agent is a uric acid synthesis inhibitor or a uricosuric agent.

81. A method according to claim 80, wherein said second therapeutic agent is a uric acid synthesis inhibitor.

82. A method according to claim 81, wherein said uric acid synthesis inhibitor is a xanthine oxidase inhibitor.

83. A method according to claim 82, wherein said xanthine oxidase inhibitor is allopurinol, febuxostat, oxypurinol, tisopurine or an inositol.

84. A method according to claim 83, wherein said xanthine oxidase inhibitor is allopurinol.

85. A method according to claim 84, wherein said allopurinol is administered in an amount from about 100 mg/day to about 300 mg/day.

86. A method according to claim 83, wherein said xanthine oxidase inhibitor is febuxostat.

87. A method according to claim 86, wherein said febuxostat is administered in an amount from about 40 mg/day to about 80 mg/day.

88. A method according to claim 80, wherein said second therapeutic agent is a uricosuric agent.

89. A method according to claim 88, wherein said uricosuric agent is probenecid, benzbromarone, sulfinpyrazone, guaifenesin, losartan, atorvastatin, amlodipine, adrenocorticotropic hormone (ACTH) or fenofibrate.

90. A method of decreasing serum uric acid level in a subject comprising administering to a subject in need thereof a pharmaceutical composition comprising:
(a) a first therapeutic agent, wherein said first therapeutic agent is a compound of formula II: 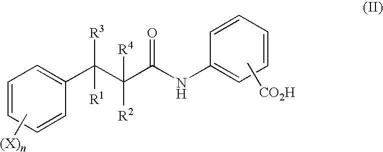
or a pharmaceutically acceptable salt thereof,
wherein each of R¹ and R² is independently selected from a hydrogen atom or a C₁-C₄ alkyl group, R³ and R⁴ are each hydrogen atoms or together form another chemical bond, each X is independently selected from a hydroxyl group, a halogen atom, a C₁-C₄ alkyl group or a C₁-C₄ alkoxy group, or when two X groups are alkyl or alkoxy groups, they may be connected together to form a ring, and n is an integer from 1 to 3;
(b) a second therapeutic agent, wherein said second therapeutic agent is a uric acid synthesis inhibitor or a uricosuric agent; and
(c) a pharmaceutically acceptable diluent or carrier.

91. A method according to claim 90, wherein said subject has hyperuricemia or gout.

92. A method according to claim 90, wherein said second therapeutic agent is a uric acid synthesis inhibitor.

93. A method according to claim 92, wherein said uric acid synthesis inhibitor is allopurinol.

94. A method for treating hypericemia comprising administering to a subject in need thereof a pharmaceutical composition comprising:
(a) a first therapeutic agent, wherein said first therapeutic agent is a compound of formula II: 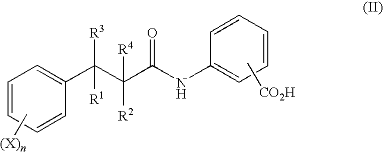
or a pharmaceutically acceptable salt thereof,
wherein each of R¹ and R² is independently selected from a hydrogen atom or a C₁-C₄ alkyl group, R³ and R⁴ are each hydrogen atoms or together form another chemical bond, each X is independently selected from a hydroxyl group, a halogen atom, a C₁-C₄ alkyl group or a C₁-C₄ alkoxy group, or when two X groups are alkyl or alkoxy groups, they may be connected together to form a ring, and n is an integer from 1 to 3;
(b) a second therapeutic agent, wherein said second therapeutic agent is a uric acid synthesis inhibitor or a uricosuric agent; and
(c) a pharmaceutically acceptable diluent or carrier.

95. A method according to claim 94, wherein said first therapeutic agent is selected from the group consisting of 2-[[3-(2-methylphenyl)-1-oxo-2-propenyl]amino]benzoic acid; 2-[[3-(3-methylphenyl)-1-oxo-2-propenyl]amino]benzoic acid; 2-[[3-(4-methylphenyl)-1-oxo-2-propenyl]amino]benzoic acid; 2-[[3-(2-ethylphenyl)-1-oxo-2-propenyl]amino]benzoic acid; 2-[[3-(3-ethylphenyl)-1-oxo-2-propenyl]amino]benzoic acid; 2-[[3-(4-ethylphenyl)-1-oxo-2-propenyl]amino]benzoic acid; 2-[[3-(2-propylphenyl)-1-oxo-2-propenyl]amino]benzoic acid; 2-[[3-(3-propylphenyl)-1-oxo-2-propenyl]amino]benzoic acid; 2-[[3-(4-propylphenyl)-1-oxo-2-propenyl]amino]benzoic acid; 2-[[3-(2-hydroxyphenyl)-1-oxo-2-propenyl]amino]benzoic acid; 2-[[3-(3-hydroxyphenyl)-1-oxo-2-propenyl]amino]benzoic acid; 2-[[3-(4-hydroxyphenyl)-1-oxo-2-propenyl]amino]benzoic acid; 2-[[3-(2-chlorophenyl)-1-oxo-2-propenyl]amino]benzoic acid; 2-[[3-(3-chlorophenyl)-1-oxo-2-propenyl]amino]benzoic acid; 2-[[3-(4-chlorophenyl)-1-oxo-2-propenyl]amino]benzoic acid; 2-[[3-(2-fluorophenyl)-1-oxo-2-propenyl]amino]benzoic acid; 2-[[3-(3-fluorophenyl)-1-oxo-2-propenyl]amino]benzoic acid; 2-[[3-(4-fluorophenyl)-1-oxo-2-propenyl]amino]benzoic acid; 2-[[3-(2-bromophenyl)-1-oxo-2-propenyl]amino]benzoic acid; 2-[[3-(3-bromophenyl)-1-oxo-2-propenyl]amino]benzoic acid; 2-[[3-(4-bromophenyl)-1-oxo-2-propenyl]amino]benzoic acid; 2-[[3-(2,3-dimethoxyphenyl)-1-oxo-2-propenyl]amino]benzoic acid; 2-[[3-(3,4-dimethoxyphenyl)-1-oxo-2-propenyl]amino]benzoic acid; 2-[[3-(2,4-dimethoxyphenyl)-1-oxo-2-propenyl]amino]benzoic acid; 2-[[3-(2,3-dimethylphenyl)-1-oxo-2-propenyl]amino]benzoic acid; 2-[[3-(3,4-dimethylphenyl)-1-oxo-2-propenyl]amino]benzoic acid; 2-[[3-(2,4-dimethylphenyl)-1-oxo-2-propenyl]amino]benzoic acid; 2-[[3-(2,3-diethoxyphenyl)-1-oxo-2-propenyl]amino]benzoic acid; 2-[[3-(3,4-diethoxyphenyl)-1-oxo-2-propenyl]amino]benzoic acid; 2-[[3-(2,4-diethoxyphenyl)-1-oxo-2-propenyl]amino]benzoic acid; 2-[[3-(2,3-dipropoxyphenyl)-1-oxo-2-propenyl]amino]benzoic acid; 2-[[3-(3,4-dipropoxyphenyl)-1-oxo-2-propenyl]amino]benzoic acid; 2-[[3-(2,4-dipropoxyphenyl)-1-oxo-2-propenyl]amino]benzoic acid; 2-[[3-(2,3-diethylphenyl)-1-oxo-2-propenyl]amino]benzoic acid; 2-[[3-(3,4-diethylphenyl)-1-oxo-2-propenyl]amino]benzoic acid; 2-[[3-(2,4-diethylphenyl)-1-oxo-2-propenyl]amino]benzoic acid; 2-[[3-(2,3-dipropylphenyl)-1-oxo-2-propenyl]amino]benzoic acid; 2-[[3-(3,4-dipropylphenyl)-1-oxo-2-propenyl]amino]benzoic acid; 2-[[3-(2,4-dipropylphenyl)-1-oxo-2-propenyl]amino]benzoic acid; 2-[[3-(2-methoxy-3-methylphenyl)-1-oxo-2-propenyl]amino]benzoic acid, 2-[[3-(3-methoxy-4-methylphenyl)-1-oxo-2-propenyl]amino]benzoic acid; 2-[[3-(2-methoxy-3-methylphenyl)-1-oxo-2-propenyl]amino]benzoic acid; 2-[[3-(2-methoxy-4-methylphenyl)-1-oxo-2-propenyl]amino]benzoic acid; 2-[[3-(2-methoxy-3-chlorophenyl)-1-oxo-2-propenyl]amino]benzoic acid; 2-[[3-(3-methoxy-4-chlorophenyl)-1-oxo-2-propenyl]amino]benzoic acid; 2-[[3-(2-methoxy-3-chlorophenyl)-1-oxo-2-propenyl]amino]benzoic acid; 2-[[3-(2-methoxy-4-chlorophenyl)-1-oxo-2-propenyl]amino]benzoic acid; 2-[[3-(2-methoxy-3-hydroxyphenyl)-1-oxo-2-propenyl]amino]benzoic acid; 2-[[3-(3-methoxy-4-hydroxyphenyl)-1-oxo-2-propenyl]amino]benzoic acid; 2-[[3-(2-methoxy-3-hydroxyphenyl)-1-oxo-2-propenyl]amino]benzoic acid; 2-[[3-(2-methoxy-4-hydroxyphenyl)-1-oxo-2-propenyl]amino]benzoic acid; 2-[[3-(3,4-trimethylenephenyl)-1-oxo-2-propenyl]amino]benzoic acid; 2-[[3-(2,3-trimethylenephenyl)-1-oxo-2-propenyl]amino]benzoic acid; 2-[[3-(3,4-methylenedioxyphenyl)-1-oxo-2-propenyl]amino]benzoic acid; and 2-[[3-(3,4-ethylenedioxyphenyl)-1-oxo-2-propenyl]amino]benzoic acid.

96. A method according to claim 95, wherein said compound of formula II is 2-[[3-(3,4-dimethoxyphenyl)-1-oxo-2-propenyl]amino]benzoic acid (tranilast).

97. A method according to claim 96, wherein said second therapeutic agent is a uric acid synthesis inhibitor.

98. A method according to claim 87, wherein said uric acid synthesis inhibitor is a xanthine oxidase inhibitor.

99. A method according to claim 98, wherein said xanthine oxidase inhibitor is allopurinol, febuxostat, oxypurinol, tisopurine or an inositol.

100. A method according to claim 99, wherein said xanthine oxidase inhibitor is allopurinol or febuxostat.

101. A method according to claim 100, wherein said xanthine oxidase inhibitor is allopurinol.

102. A method for treating gout comprising administering to a subject in need thereof a pharmaceutical composition comprising:
(a) a first therapeutic agent, wherein said first therapeutic agent is a compound of formula II: 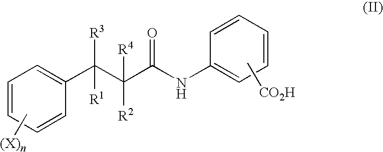
or a pharmaceutically acceptable salt thereof,
wherein each of R¹ and R² is independently selected from a hydrogen atom or a C₁-C₄ alkyl group, R³ and R⁴ are each hydrogen atoms or together form another chemical bond, each X is independently selected from a hydroxyl group, a halogen atom, a C₁-C₄ alkyl group or a C₁-C₄ alkoxy group, or when two X groups are alkyl or alkoxy groups, they may be connected together to form a ring, and n is an integer from 1 to 3;
(b) a second therapeutic agent, wherein said second therapeutic agent is a uric acid synthesis inhibitor or a uricosuric agent; and
(c) a pharmaceutically acceptable diluent or carrier.

103. A method according to claim 102, wherein said first therapeutic agent is selected from the group consisting of 2-[[3-(2-methylphenyl)-1-oxo-2-propenyl]amino]benzoic acid; 2-[[3-(3-methylphenyl)-1-oxo-2-propenyl]amino]benzoic acid; 2-[[3-(4-methylphenyl)-1-oxo-2-propenyl]amino]benzoic acid; 2-[[3-(2-ethylphenyl)-1-oxo-2-propenyl]amino]benzoic acid; 2-[[3-(3-ethylphenyl)-1-oxo-2-propenyl]amino]benzoic acid; 2-[[3-(4-ethylphenyl)-1-oxo-2-propenyl]amino]benzoic acid; 2-[[3-(2-propylphenyl)-1-oxo-2-propenyl]amino]benzoic acid; 2-[[3-(3-propylphenyl)-1-oxo-2-propenyl]amino]benzoic acid; 2-[[3-(4-propylphenyl)-1-oxo-2-propenyl]amino]benzoic acid; 2-[[3-(2-hydroxyphenyl)-1-oxo-2-propenyl]amino]benzoic acid; 2-[[3-(3-hydroxyphenyl)-1-oxo-2-propenyl]amino]benzoic acid; 2-[[3-(4-hydroxyphenyl)-1-oxo-2-propenyl]amino]benzoic acid; 2-[[3-(2-chlorophenyl)-1-oxo-2-propenyl]amino]benzoic acid; 2-[[3-(3-chlorophenyl)-1-oxo-2-propenyl]amino]benzoic acid; 2-[[3-(4-chlorophenyl)-1-oxo-2-propenyl]amino]benzoic acid; 2-[[3-(2-fluorophenyl)-1-oxo-2-propenyl]amino]benzoic acid; 2-[[3-(3-fluorophenyl)-1-oxo-2-propenyl]amino]benzoic acid; 2-[[3-(4-fluorophenyl)-1-oxo-2-propenyl]amino]benzoic acid; 2-[[3-(2-bromophenyl)-1-oxo-2-propenyl]amino]benzoic acid; 2-[[3-(3-bromophenyl)-1-oxo-2-propenyl]amino]benzoic acid; 2-[[3-(4-bromophenyl)-1-oxo-2-propenyl]amino]benzoic acid; 2-[[3-(2,3-dimethoxyphenyl)-1-oxo-2-propenyl]amino]benzoic acid; 2-[[3-(3,4-dimethoxyphenyl)-1-oxo-2-propenyl]amino]benzoic acid; 2-[[3-(2,4-dimethoxyphenyl)-1-oxo-2-propenyl]amino]benzoic acid; 2-[[3-(2,3-dimethylphenyl)-1-oxo-2-propenyl]amino]benzoic acid; 2-[[3-(3,4-dimethylphenyl)-1-oxo-2-propenyl]amino]benzoic acid; 2-[[3-(2,4-dimethylphenyl)-1-oxo-2-propenyl]amino]benzoic acid; 2-[[3-(2,3-diethoxyphenyl)-1-oxo-2-propenyl]amino]benzoic acid; 2-[[3-(3,4-diethoxyphenyl)-1-oxo-2-propenyl]amino]benzoic acid; 2-[[3-(2,4-diethoxyphenyl)-1-oxo-2-propenyl]amino]benzoic acid; 2-[[3-(2,3-dipropoxyphenyl)-1-oxo-2-propenyl]amino]benzoic acid; 2-[[3-(3,4-dipropoxyphenyl)-1-oxo-2-propenyl]amino]benzoic acid; 2-[[3-(2,4-dipropoxyphenyl)-1-oxo-2-propenyl]amino]benzoic acid; 2-[[3-(2,3-diethylphenyl)-1-oxo-2-propenyl]amino]benzoic acid; 2-[[3-(3,4-diethylphenyl)-1-oxo-2-propenyl]amino]benzoic acid; 2-[[3-(2,4-diethylphenyl)-1-oxo-2-propenyl]amino]benzoic acid; 2-[[3-(2,3-dipropylphenyl)-1-oxo-2-propenyl]amino]benzoic acid; 2-[[3-(3,4-dipropylphenyl)-1-oxo-2-propenyl]amino]benzoic acid; 2-[[3-(2,4-dipropylphenyl)-1-oxo-2-propenyl]amino]benzoic acid; 2-[[3-(2-methoxy-3-methylphenyl)-1-oxo-2-propenyl]amino]benzoic acid, 2-[[3-(3-methoxy-4-methylphenyl)-1-oxo-2-propenyl]amino]benzoic acid; 2-[[3-(2-methoxy-3-methylphenyl)-1-oxo-2-propenyl]amino]benzoic acid; 2-[[3-(2-methoxy-4-methylphenyl)-1-oxo-2-propenyl]amino]benzoic acid; 2-[[3-(2-methoxy-3-chlorophenyl)-1-oxo-2-propenyl]amino]benzoic acid; 2-[[3-(3-methoxy-4-chlorophenyl)-1-oxo-2-propenyl]amino]benzoic acid; 2-[[3-(2-methoxy-3-chlorophenyl)-1-oxo-2-propenyl]amino]benzoic acid; 2-[[3-(2-methoxy-4-chlorophenyl)-1-oxo-2-propenyl]amino]benzoic acid; 2-[[3-(2-methoxy-3-hydroxyphenyl)-1-oxo-2-propenyl]amino]benzoic acid; 2-[[3-(3-methoxy-4-hydroxyphenyl)-1-oxo-2-propenyl]amino]benzoic acid; 2-[[3-(2-methoxy-3-hydroxyphenyl)-1-oxo-2-propenyl]amino]benzoic acid; 2-[[3-(2-methoxy-4-hydroxyphenyl)-1-oxo-2-propenyl]amino]benzoic acid; 2-[[3-(3,4-trimethylenephenyl)-1-oxo-2-propenyl]amino]benzoic acid; 2-[[3-(2,3-trimethylenephenyl)-1-oxo-2-propenyl]amino]benzoic acid; 2-[[3-(3,4-methylenedioxyphenyl)-1-oxo-2-propenyl]amino]benzoic acid; and 2-[[3-(3,4-ethylenedioxyphenyl)-1-oxo-2-propenyl]amino]benzoic acid.

104. A method according to claim 103, wherein said compound of formula II is 2-[[3-(3,4-dimethoxyphenyl)-1-oxo-2-propenyl]amino]benzoic acid (tranilast).

105. A method according to claim 104, wherein said second therapeutic agent is a uric acid synthesis inhibitor.

106. A method according to claim 105, wherein said uric acid synthesis inhibitor is a xanthine oxidase inhibitor.

107. A method according to claim 106, wherein said xanthine oxidase inhibitor is allopurinol, febuxostat, oxypurinol, tisopurine or an inositol.

108. A method according to claim 107, wherein said xanthine oxidase inhibitor is allopurinol or febuxostat.

109. A method according to claim 108, wherein said xanthine oxidase inhibitor is allopurinol.

110. A method for treating hypericemia comprising administering to a subject in need thereof a pharmaceutically effective amount of a compound of formula II: 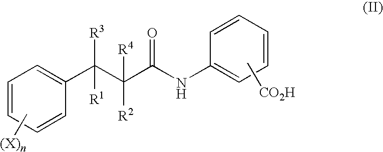
or a pharmaceutically acceptable salt thereof,
wherein each of R¹ and R² is independently selected from a hydrogen atom or a C₁-C₄ alkyl group, R³ and R⁴ are each hydrogen atoms or together form another chemical bond, each X is independently selected from a hydroxyl group, a halogen atom, a C₁-C₄ alkyl group or a C₁-C₄ alkoxy group, or when two X groups are alkyl or alkoxy groups, they may be connected together to form a ring, and n is an integer from 1 to 3.

111. A method according to claim 110, wherein said compound of formula II is selected from the group consisting of 2-[[3-(2-methylphenyl)-1-oxo-2-propenyl]amino]benzoic acid; 2-[[3-(3-methylphenyl)-1-oxo-2-propenyl]amino]benzoic acid; 2-[[3-(4-methylphenyl)-1-oxo-2-propenyl]amino]benzoic acid; 2-[[3-(2-ethylphenyl)-1-oxo-2-propenyl]amino]benzoic acid; 2-[[3-(3-ethylphenyl)-1-oxo-2-propenyl]amino]benzoic acid; 2-[[3-(4-ethylphenyl)-1-oxo-2-propenyl]amino]benzoic acid; 2-[[3-(2-propylphenyl)-1-oxo-2-propenyl]amino]benzoic acid; 2-[[3-(3-propylphenyl)-1-oxo-2-propenyl]amino]benzoic acid; 2-[[3-(4-propylphenyl)-1-oxo-2-propenyl]amino]benzoic acid; 2-[[3-(2-hydroxyphenyl)-1-oxo-2-propenyl]amino]benzoic acid; 2-[[3-(3-hydroxyphenyl)-1-oxo-2-propenyl]amino]benzoic acid; 2-[[3-(4-hydroxyphenyl)-1-oxo-2-propenyl]amino]benzoic acid; 2-[[3-(2-chlorophenyl)-1-oxo-2-propenyl]amino]benzoic acid; 2-[[3-(3-chlorophenyl)-1-oxo-2-propenyl]amino]benzoic acid; 2-[[3-(4-chlorophenyl)-1-oxo-2-propenyl]amino]benzoic acid; 2-[[3-(2-fluorophenyl)-1-oxo-2-propenyl]amino]benzoic acid; 2-[[3-(3-fluorophenyl)-1-oxo-2-propenyl]amino]benzoic acid; 2-[[3-(4-fluorophenyl)-1-oxo-2-propenyl]amino]benzoic acid; 2-[[3-(2-bromophenyl)-1-oxo-2-propenyl]amino]benzoic acid; 2-[[3-(3-bromophenyl)-1-oxo-2-propenyl]amino]benzoic acid; 2-[[3-(4-bromophenyl)-1-oxo-2-propenyl]amino]benzoic acid; 2-[[3-(2,3-dimethoxyphenyl)-1-oxo-2-propenyl]amino]benzoic acid; 2-[[3-(3,4-dimethoxyphenyl)-1-oxo-2-propenyl]amino]benzoic acid; 2-[[3-(2,4-dimethoxyphenyl)-1-oxo-2-propenyl]amino]benzoic acid; 2-[[3-(2,3-dimethylphenyl)-1-oxo-2-propenyl]amino]benzoic acid; 2-[[3-(3,4-dimethylphenyl)-1-oxo-2-propenyl]amino]benzoic acid; 2-[[3-(2,4-dimethylphenyl)-1-oxo-2-propenyl]amino]benzoic acid; 2-[[3-(2,3-diethoxyphenyl)-1-oxo-2-propenyl]amino]benzoic acid; 2-[[3-(3,4-diethoxyphenyl)-1-oxo-2-propenyl]amino]benzoic acid; 2-[[3-(2,4-diethoxyphenyl)-1-oxo-2-propenyl]amino]benzoic acid; 2-[[3-(2,3-dipropoxyphenyl)-1-oxo-2-propenyl]amino]benzoic acid; 2-[[3-(3,4-dipropoxyphenyl)-1-oxo-2-propenyl]amino]benzoic acid; 2-[[3-(2,4-dipropoxyphenyl)-1-oxo-2-propenyl]amino]benzoic acid; 2-[[3-(2,3-diethylphenyl)-1-oxo-2-propenyl]amino]benzoic acid; 2-[[3-(3,4-diethylphenyl)-1-oxo-2-propenyl]amino]benzoic acid; 2-[[3-(2,4-diethylphenyl)-1-oxo-2-propenyl]amino]benzoic acid; 2-[[3-(2,3-dipropylphenyl)-1-oxo-2-propenyl]amino]benzoic acid; 2-[[3-(3,4-dipropylphenyl)-1-oxo-2-propenyl]amino]benzoic acid; 2-[[3-(2,4-dipropylphenyl)-1-oxo-2-propenyl]amino]benzoic acid; 2-[[3-(2-methoxy-3-methylphenyl)-1-oxo-2-propenyl]amino]benzoic acid, 2-[[3-(3-methoxy-4-methylphenyl)-1-oxo-2-propenyl]amino]benzoic acid; 2-[[3-(2-methoxy-3-methylphenyl)-1-oxo-2-propenyl]amino]benzoic acid; 2-[[3-(2-methoxy-4-methylphenyl)-1-oxo-2-propenyl]amino]benzoic acid; 2-[[3-(2-methoxy-3-chlorophenyl)-1-oxo-2-propenyl]amino]benzoic acid; 2-[[3-(3-methoxy-4-chlorophenyl)-1-oxo-2-propenyl]amino]benzoic acid; 2-[[3-(2-methoxy-3-chlorophenyl)-1-oxo-2-propenyl]amino]benzoic acid; 2-[[3-(2-methoxy-4-chlorophenyl)-1-oxo-2-propenyl]amino]benzoic acid; 2-[[3-(2-methoxy-3-hydroxyphenyl)-1-oxo-2-propenyl]amino]benzoic acid; 2-[[3-(3-methoxy-4-hydroxyphenyl)-1-oxo-2-propenyl]amino]benzoic acid; 2-[[3-(2-methoxy-3-hydroxyphenyl)-1-oxo-2-propenyl]amino]benzoic acid; 2-[[3-(2-methoxy-4-hydroxyphenyl)-1-oxo-2-propenyl]amino]benzoic acid; 2-[[3-(3,4-trimethylenephenyl)-1-oxo-2-propenyl]amino]benzoic acid; 2-[[3-(2,3-trimethylenephenyl)-1-oxo-2-propenyl]amino]benzoic acid; 2-[[3-(3,4-methylenedioxyphenyl)-1-oxo-2-propenyl]amino]benzoic acid; and 2-[[3-(3,4-ethylenedioxyphenyl)-1-oxo-2-propenyl]amino]benzoic acid.

112. A method according to claim 111, wherein said compound of formula II is 2-[[3-(3,4-dimethoxyphenyl)-1-oxo-2-propenyl]amino]benzoic acid (tranilast).

113. A method according to claim 112, further comprising administering to said subject a second therapeutic agent, wherein said second therapeutic agent is a uric acid synthesis inhibitor or a uricosuric agent.

114. A method according to claim 113, wherein said second therapeutic agent is a uric acid synthesis inhibitor.

115. A method according to claim 114, wherein said uric acid synthesis inhibitor is a xanthine oxidase inhibitor.

116. A method according to claim 115, wherein said xanthine oxidase inhibitor is allopurinol, febuxostat, oxypurinol, tisopurine or an inositol.

117. A method according to claim 116, wherein said xanthine oxidase inhibitor is allopurinol or febuxostat.

118. A method according to claim 117, wherein said xanthine oxidase inhibitor is allopurinol.

119. A method for treating gout comprising administering to a subject in need thereof a pharmaceutically effective amount of a compound of formula II: 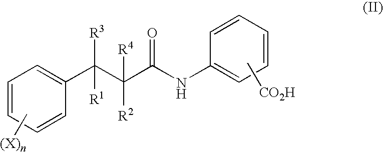
or a pharmaceutically acceptable salt thereof,
wherein each of R¹ and R² is independently selected from a hydrogen atom or a C₁-C₄ alkyl group, R³ and R⁴ are each hydrogen atoms or together form another chemical bond, each X is independently selected from a hydroxyl group, a halogen atom, a C₁-C₄ alkyl group or a C₁-C₄ alkoxy group, or when two X groups are alkyl or alkoxy groups, they may be connected together to form a ring, and n is an integer from 1 to 3.

120. A method according to claim 119, wherein said compound of formula II is selected from the group consisting of 2-[[3-(2-methylphenyl)-1-oxo-2-propenyl]amino]benzoic acid; 2-[[3-(3-methylphenyl)-1-oxo-2-propenyl]amino]benzoic acid; 2-[[3-(4-methylphenyl)-1-oxo-2-propenyl]amino]benzoic acid; 2-[[3-(2-ethylphenyl)-1-oxo-2-propenyl]amino]benzoic acid; 2-[[3-(3-ethylphenyl)-1-oxo-2-propenyl]amino]benzoic acid; 2-[[3-(4-ethylphenyl)-1-oxo-2-propenyl]amino]benzoic acid; 2-[[3-(2-propylphenyl)-1-oxo-2-propenyl]amino]benzoic acid; 2-[[3-(3-propylphenyl)-1-oxo-2-propenyl]amino]benzoic acid; 2-[[3-(4-propylphenyl)-1-oxo-2-propenyl]amino]benzoic acid; 2-[[3-(2-hydroxyphenyl)-1-oxo-2-propenyl]amino]benzoic acid; 2-[[3-(3-hydroxyphenyl)-1-oxo-2-propenyl]amino]benzoic acid; 2-[[3-(4-hydroxyphenyl)-1-oxo-2-propenyl]amino]benzoic acid; 2-[[3-(2-chlorophenyl)-1-oxo-2-propenyl]amino]benzoic acid; 2-[[3-(3-chlorophenyl)-1-oxo-2-propenyl]amino]benzoic acid; 2-[[3-(4-chlorophenyl)-1-oxo-2-propenyl]amino]benzoic acid; 2-[[3-(2-fluorophenyl)-1-oxo-2-propenyl]amino]benzoic acid; 2-[[3-(3-fluorophenyl)-1-oxo-2-propenyl]amino]benzoic acid; 2-[[3-(4-fluorophenyl)-1-oxo-2-propenyl]amino]benzoic acid; 2-[[3-(2-bromophenyl)-1-oxo-2-propenyl]amino]benzoic acid; 2-[[3-(3-bromophenyl)-1-oxo-2-propenyl]amino]benzoic acid; 2-[[3-(4-bromophenyl)-1-oxo-2-propenyl]amino]benzoic acid; 2-[[3-(2,3-dimethoxyphenyl)-1-oxo-2-propenyl]amino]benzoic acid; 2-[[3-(3,4-dimethoxyphenyl)-1-oxo-2-propenyl]amino]benzoic acid; 2-[[3-(2,4-dimethoxyphenyl)-1-oxo-2-propenyl]amino]benzoic acid; 2-[[3-(2,3-dimethylphenyl)-1-oxo-2-propenyl]amino]benzoic acid; 2-[[3-(3,4-dimethylphenyl)-1-oxo-2-propenyl]amino]benzoic acid; 2-[[3-(2,4-dimethylphenyl)-1-oxo-2-propenyl]amino]benzoic acid; 2-[[3-(2,3-diethoxyphenyl)-1-oxo-2-propenyl]amino]benzoic acid; 2-[[3-(3,4-diethoxyphenyl)-1-oxo-2-propenyl]amino]benzoic acid; 2-[[3-(2,4-diethoxyphenyl)-1-oxo-2-propenyl]amino]benzoic acid; 2-[[3-(2,3-dipropoxyphenyl)-1-oxo-2-propenyl]amino]benzoic acid; 2-[[3-(3,4-dipropoxyphenyl)-1-oxo-2-propenyl]amino]benzoic acid; 2-[[3-(2,4-dipropoxyphenyl)-1-oxo-2-propenyl]amino]benzoic acid; 2-[[3-(2,3-diethylphenyl)-1-oxo-2-propenyl]amino]benzoic acid; 2-[[3-(3,4-diethylphenyl)-1-oxo-2-propenyl]amino]benzoic acid; 2-[[3-(2,4-diethylphenyl)-1-oxo-2-propenyl]amino]benzoic acid; 2-[[3-(2,3-dipropylphenyl)-1-oxo-2-propenyl]amino]benzoic acid; 2-[[3-(3,4-dipropylphenyl)-1-oxo-2-propenyl]amino]benzoic acid; 2-[[3-(2,4-dipropylphenyl)-1-oxo-2-propenyl]amino]benzoic acid; 2-[[3-(2-methoxy-3-methylphenyl)-1-oxo-2-propenyl]amino]benzoic acid, 2-[[3-(3-methoxy-4-methylphenyl)-1-oxo-2-propenyl]amino]benzoic acid; 2-[[3-(2-methoxy-3-methylphenyl)-1-oxo-2-propenyl]amino]benzoic acid; 2-[[3-(2-methoxy-4-methylphenyl)-1-oxo-2-propenyl]amino]benzoic acid; 2-[[3-(2-methoxy-3-chlorophenyl)-1-oxo-2-propenyl]amino]benzoic acid; 2-[[3-(3-methoxy-4-chlorophenyl)-1-oxo-2-propenyl]amino]benzoic acid; 2-[[3-(2-methoxy-3-chlorophenyl)-1-oxo-2-propenyl]amino]benzoic acid; 2-[[3-(2-methoxy-4-chlorophenyl)-1-oxo-2-propenyl]amino]benzoic acid; 2-[[3-(2-methoxy-3-hydroxyphenyl)-1-oxo-2-propenyl]amino]benzoic acid; 2-[[3-(3-methoxy-4-hydroxyphenyl)-1-oxo-2-propenyl]amino]benzoic acid; 2-[[3-(2-methoxy-3-hydroxyphenyl)-1-oxo-2-propenyl]amino]benzoic acid; 2-[[3-(2-methoxy-4-hydroxyphenyl)-1-oxo-2-propenyl]amino]benzoic acid; 2-[[3-(3,4-trimethylenephenyl)-1-oxo-2-propenyl]amino]benzoic acid; 2-[[3-(2,3-trimethylenephenyl)-1-oxo-2-propenyl]amino]benzoic acid; 2-[[3-(3,4-methylenedioxyphenyl)-1-oxo-2-propenyl]amino]benzoic acid; and 2-[[3-(3,4-ethylenedioxyphenyl)-1-oxo-2-propenyl]amino]benzoic acid.

121. A method according to claim 120, wherein said compound of formula II is 2-[[3-(3,4-dimethoxyphenyl)-1-oxo-2-propenyl]amino]benzoic acid (tranilast).

122. A method according to claim 121, further comprising administering to said subject a second therapeutic agent, wherein said second therapeutic agent is a uric acid synthesis inhibitor or a uricosuric agent.

123. A method according to claim 122, wherein said second therapeutic agent is a uric acid synthesis inhibitor.

124. A method according to claim 123 wherein said uric acid synthesis inhibitor is a xanthine oxidase inhibitor.

125. A method according to claim 124, wherein said xanthine oxidase inhibitor is allopurinol, febuxostat, oxypurinol, tisopurine or an inositol.

126. A method according to claim 125, wherein said xanthine oxidase inhibitor is allopurinol or febuxostat.

127. A method according to claim 126, wherein said xanthine oxidase inhibitor is allopurinol.
